(19) United States
(12) Patent Application Publication
(10) Pub. No.: US 2024/0016777 A1
(43) Pub. Date: Jan. 18, 2024

(54) COMPOUNDS AND METHODS FOR TREATING VIRAL INFECTIONS

(71) Applicant: Beth Israel Deaconess Medical Center, Inc., Boston, MA (US)

(72) Inventors: Robert Flaumenhaft, Newton, MA (US); Lin Lin, Brookline, MA (US)

(21) Appl. No.: 18/252,502

(22) PCT Filed: Nov. 12, 2021
(86) PCT No.: PCT/US2021/059263

§ 371 Date: May 10, 2023

Related U.S. Application Data
(60) Provisional application No. 63/112,857, filed on Nov. 12, 2020.

(51) Int. Cl. A61K 31/353; A61K 9/00; A61K 31/7048; A61K 45/06; A61P 31/14; A61P 7/02

(52) U.S. Cl. CPC A61K 31/353; A61K 9/0043; A61K 31/7048; A61K 45/06; A61P 31/14; A61P 7/02

(57) ABSTRACT

The present application provides a compound of Formula (I), or a pharmaceutically acceptable salt thereof. Pharmaceutical compositions containing the compound of Formula (I), and methods of using the compound of Formula (I) for treating viral infections and inhibiting thromobosis are also provided.

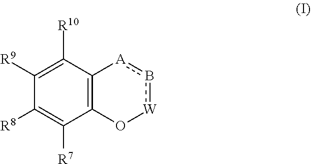

### CLAIM OF PRIORITY

This application claims priority to U.S. Patent Application Ser. No. 63/112,857, filed on Nov. 12, 2020, the entire contents of which are hereby incorporated by reference.

### STATEMENT REGARDING FEDERALLY SPONSORED RESEARCH OR DEVELOPMENT

This invention was made with government support under Grant No. HL135775 awarded by the National Institutes of Health. The government has certain rights in the invention.

### TECHNICAL FIELD

This invention relates to flavonoid compounds useful in treating viral infections.

### BACKGROUND

Viral infections continue to grow worldwide, representing one of the greatest burdens in human health. In 2020, more than 1,000,000 people died from Covid-19 alone. No effective treatment options exist for treating many viral infections. Therapy for Covid-19, or example, is currently dominated by supportive measures, most of which can only be administered in the hospital setting. Safe and effective oral agents to treat outpatients with Covid-19 would reduce the strain on hospitals and keep infected patients away from the very medical centers where our most vulnerable population—those with underlying illnesses—resides.

### SUMMARY

The present disclosure is based, at least in part, on a realization that flavonoid compounds containing a C₄₋₁₅ alkenyl group (such as a prenyl group or a geranyl group) in their chemical structure are inhibitors of SARS-CoV-2 main protease, and as such, the compounds are useful in treating viral infections caused by SARS-CoV-2 virus (e.g., COVID-19). Advantageously, these compounds can be sourced from abundant agricultural products, are generally orally available, and in many instances have a well-studied metabolism. Similarly, the safety profile of many flavonoids, including prenylated flavonoids, is well-established and a large number of these compounds are commercially available as over-the-counter nutraceuticals.

In a general aspect, the present disclosure provides a method of modulating activity of a main protease of a virus selected from SARS-CoV, SAR-CoV-2, and MERS-CoV in a cell, the method comprising contacting the cell with an effective amount of a compound of Formula (I):

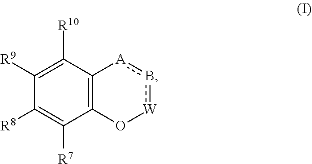

or a pharmaceutically acceptable salt thereof, wherein:
A is CR¹R²;
B is CR³R⁴;
W is CR⁵R⁶;
R¹, R², R³, R⁴, R⁵, R⁶, R⁷, R⁸, R⁹, and R¹⁰ are each independently selected from H, Cy^{A}, halo, CN, NO₂, C₁₋₆ alkyl, C₁₋₆ haloalkyl, C₄₋₁₅ alkenyl, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl and C₄₋₁₅ alkenyl are each optionally substituted with 1, 2 or 3 substituents independently selected from Cy^{A}, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1} and S(O)₂NR^{c1}R^{d1};
each [symbol] bond is either a single bond or a double bond, provided that:
(i) when the bond between A and B is a double bond, then R² and R⁴ are absent and the bond between B and W is a single bond; and
(ii) when the bond between B and W is a double bond, then R⁴ and R⁶ are absent and the bond between A and B is a single bond;
or R¹ and R² together form an oxo group;
or R³ and R⁵, together with the carbon atoms to which they are attached, form 4-10 membered heterocycloalkyl, which is optionally substituted with 1, 2, 3, 4, or 5 substituents independently selected from R^{Cy};
or R¹ and R³, together with the carbon atoms to which they are attached, form 4-10 membered heterocycloalkyl, which is optionally substituted with 1, 2, 3, 4, or 5 substituents independently selected from R^{Cy};
R⁷ and R⁸, together with the carbon atoms to which they are attached, form a 4-10 membered heterocycloalkyl, which is optionally substituted with 1, 2, 3, or 4 substituents independently selected from R^{Cy};
R⁸ and R⁹, together with the carbon atoms to which they are attached, form a 4-10 membered heterocycloalkyl, which is optionally substituted with 1, 2, 3, or 4 substituents independently selected from R^{Cy};
R⁹ and R¹⁰, together with the carbon atoms to which they are attached, form a 4-10 membered heterocycloalkyl, which is optionally substituted with 1, 2, 3, or 4 substituents independently selected from R^{Cy};
each R^{a1}, R^{b1}, R^{c1}, and R^{d1} is independently selected from H, Cy^{A}, C₁₋₆ alkyl, C₄₋₁₅ alkenyl, C₁₋₆ haloalkyl, wherein said C₁₋₆ alkyl and C₄₋₁₅ alkenyl are each optionally substituted with 1, 2, or 3 substituents independently selected from R^{g};
each Cy^{A} is independently selected from C₆₋₁₀ aryl, C₃₋₈ cycloalkyl, and 4-10 membered heterocycloalkyl, each of which is optionally substituted with 1, 2, 3, 4, or 5 substituents independently selected from R^{Cy};
each R^{Cy} is independently selected from halo, C₁₋₆ alkyl, C₁₋₆ haloalkyl, C₆₋₁₀ aryl, C₄₋₁₅ alkenyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}, wherein said C₁₋₆ alkyl, C₄₋₁₅ alkenyl, and C₆₋₁₀ aryl are each optionally substituted by 1, 2, or 3 substituents independently selected from C₁₋₆ alkyl, C₁₋₆ haloalkyl, halo, CN, NO₂, OR², SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
each R^{a2}, R^{b2}, R^{c2}, and R^{d2} is independently selected from H, C₁₋₆ alkyl, C₄₋₁₅ alkenyl, C₆₋₁₀ aryl, and 4-10 membered heterocycloalkyl, each of which is optionally substituted with 1, 2, 3, 4, or 5 substituents independently selected from R^{A};
each R^{A} is independently selected from C₁₋₆ alkyl, C₁₋₆haloalkyl, halo, CN, NO₂, OR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}, wherein said C₁₋₆ alkyl is optionally substituted with 1, 2, or 3 substituents selected from CN, NO₂, OR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3};
each R^{a3}, R^{b3}, R^{c3}, and R^{d3} is independently selected from H, C₁₋₆ alkyl, C₄₋₁₅ alkenyl, C₁₋₆ haloalkyl, wherein said C₁₋₆ alkyl and C₄₋₁₅ alkenyl are each optionally substituted with 1, 2, or 3 substituents independently selected from R^{g};
or any R^{c1} and R^{d1} together with the N atom to which they are attached form a 4-, 5-, 6-, or 7-membered heterocycloalkyl group optionally substituted with 1, 2, or 3 substituents independently selected from R^{g};
or any R^{c2} and R^{d2} together with the N atom to which they are attached form a 4-, 5-, 6-, or 7-membered heterocycloalkyl group optionally substituted with 1, 2, or 3 substituents independently selected from R^{g};
or any R^{c3} and R^{d3} together with the N atom to which they are attached form a 4-, 5-, 6-, or 7-membered heterocycloalkyl group optionally substituted with 1, 2, or 3 substituents independently selected from R^{g}; and
each R^{g} is independently selected from OH, NO₂, CN, halo, C₁₋₆ alkyl, C₁₋₄ haloalkyl, C₁₋₆ alkoxy, C₁₋₆ haloalkoxy, cyano-C₁₋₃ alkylene, HO—C₁₋₃ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino;
provided that the compound of Formula (I) comprises at least one C₄₋₁₅ alkenyl.

In some embodiments, the method comprises contacting the cell in vitro, in vivo, or ex vivo.

In some embodiments, R¹, R², R³, R⁴, R⁵, R⁶, R⁷, R⁸, R⁹, and R¹⁰ are each independently selected from H, Cy^{A}, halo, C₁₋₆ alkyl, C₁₋₆ haloalkyl, C₄₋₁₅ alkenyl, OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, and NR^{c1}S(O)₂NR^{c1}R^{d1}; wherein said C₁₋₆ alkyl and C₄₋₁₅ alkenyl are each optionally substituted with 1, 2 or 3 substituents independently selected from Cy^{A}, halo, OR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, NR^{c1}S(O)₂NR^{c1}R^{d1}, S(O)₂R^{b1}, and S(O)₂NR^{c1}R^{d1}.

In some embodiments, R¹, R², R³, R⁴, R⁵, R⁶, R⁷, R⁸, R⁹, and R¹⁰ are each independently selected from H, Cy^{A}, C₁₋₆ alkyl, C₄₋₁₅ alkenyl, and OR^{a1}, wherein said C₁₋₆ alkyl and C₄₋₁₅ alkenyl are each optionally substituted with OR^{a1}.

In some embodiments, each R^{a1}, R^{b1}, R^{c1}, and R^{d1} is independently selected from H, Cy^{A}, C₁₋₆ alkyl, and C₄₋₁₅ alkenyl.

In some embodiments, each Cy^{A} is independently selected from C₆₋₁₀ aryl, C₃₋₈ cycloalkyl, and 4-10 membered heterocycloalkyl, each of which is optionally substituted with 1, 2, or 3 substituents independently selected from R^{Cy}.

In some embodiments, each R^{Cy} is independently selected from halo, C₁₋₆ alkyl, C₆₋₁₀ aryl, C₄₋₁₅ alkenyl, OR^{a2}, C(O)R^{b2}, OC(O)R^{b2}, OC(O)NR^{c2}R^{d2}, NR^{c2}R^{d2}, NR^{c2}C(O)R^{b2}, NR^{c2}C(O)OR^{a2}, and NR^{c2}C(O)NR^{c2}R^{d2}, wherein said C₁₋₆ alkyl, C₄₋₁₅ alkenyl, and C₆₋₁₀ aryl are each optionally substituted by 1, 2, or 3 substituents independently selected from C₁₋₆ alkyl, OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, and NR^{c2}C(O)NR^{c2}R^{d2}.

In some embodiments, each R^{Cy} is independently selected from C₁₋₆ alkyl, C₆₋₁₀ aryl, C₄₋₁₅ alkenyl, OR^{a2}, C(O)R^{b2}, and OC(O)R^{b2}, wherein said C₁₋₆ alkyl, C₄₋₁₅ alkenyl, and C₆₋₁₀ aryl are each optionally substituted by 1, 2, or 3 substituents independently selected from OR^{a2} and OC(O)R^{b2}.

In some embodiments, each R^{a2}, R^{b2}, R^{c2}, and R^{d2} is independently selected from H, C₁₋₆ alkyl, C₄₋₁₅ alkenyl, C₆₋₁₀ aryl, and 4-10 membered heterocycloalkyl, each of which is optionally substituted with 1, 2, or 3 substituents independently selected from R^{A}.

In some embodiments, each R^{A} is independently selected from C₁₋₆ alkyl, OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, and NR^{c3}C(O)NR^{c3}R^{d3}, wherein said C₁₋₆ alkyl is optionally substituted with 1, 2, or 3 substituents selected from OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, and NR^{c3}C(O)NR^{c3}R^{d3}.

In some embodiments, each R^{A} is independently selected from C₁₋₆ alkyl, OR^{a3}, and OC(O)R^{b3}, wherein said C₁₋₆ alkyl is optionally substituted with 1, 2, or 3 substituents selected from OR^{a3} and OC(O)R^{b3}.

In some embodiments, each R^{a3}, R^{b3}, R^{c3}, and R^{d3} is independently selected from H, C₁₋₆ alkyl, and C₄₋₁₅ alkenyl, wherein said C₁₋₆ alkyl and C₄₋₁₅ alkenyl are each optionally substituted with 1, 2, or 3 substituents independently selected from R^{g}.

In some embodiments, each R^{g} is independently selected from OH, C₁₋₆ alkyl, C₁₋₆ alkoxy, HO—C₁₋₃ alkylene, amino, C₁₋₆ alkylamino, and di(C₁₋₆ alkyl)amino.

In some embodiments:
R¹, R², R³, R⁴, R⁵, R⁶, R⁷, R⁸, R⁹, and R¹⁰ are each independently selected from H, Cy^{A}, halo, C₁₋₆ alkyl, C₁₋₆ haloalkyl, C₄₋₁₅ alkenyl, OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, and NR^{c1}S(O)₂NR^{c1}R^{d1}; wherein said C₁₋₆ alkyl and C₄₋₁₅ alkenyl are each optionally substituted with 1, 2 or 3 substituents independently selected from Cy^{A}, halo, OR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, NR^{c1}S(O)₂NR^{c1}R^{d1}, S(O)₂R^{b1}, and S(O)₂NR^{c1}R^{d1};
each R^{a1}, R^{b1}, R^{c1}, and R^{d1} is independently selected from H, Cy^{A}, C₁₋₆ alkyl, and C₄₋₁₅ alkenyl;
each Cy^{A} is independently selected from C₆₋₁₀ aryl, C₃₋₈ cycloalkyl, and 4-10 membered heterocycloalkyl, each of which is optionally substituted with 1, 2, or 3 substituents independently selected from R^{Cy}.
each R^{Cy} is independently selected from halo, C₁₋₆ alkyl, C₆₋₁₀ aryl, C₄₋₁₅ alkenyl, OR^{a2}, C(O)R^{b2}, OC(O)R^{b2}, OC(O)NR^{c2}R^{d2}, NR^{c2}R^{d2}, NR^{c2}C(O)R^{b2}, NR^{c2}C(O)OR^{a2}, and NR^{c2}C(O)NR^{c2}R^{d2}, wherein said C₁₋₆ alkyl, C₄₋₁₅ alkenyl, and C₆₋₁₀ aryl are each optionally substituted by 1, 2, or 3 substituents independently selected from C₁₋₆ alkyl, OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, and NR^{c2}C(O)NR^{c2}R^{d2};
each R^{a2}, R^{b2}, R^{c2}, and R^{d2} is independently selected from H, C₁₋₆ alkyl, C₄₋₁₅ alkenyl, C₆₋₁₀ aryl, and 4-10 membered heterocycloalkyl, each of which is optionally substituted with 1, 2, or 3 substituents independently selected from R^{A};
each R^{A} is independently selected from C₁₋₆ alkyl, OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, and NR^{c3}C(O)NR^{c3}R^{d3}, wherein said C₁₋₆ alkyl is optionally substituted with 1, 2, or 3 substituents selected from OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, and NR^{c3}C(O)NR^{c3}R^{d3};
each R^{a3}, R^{b3}, R^{c3}, and R^{d3} is independently selected from H, C₁₋₆ alkyl, and C₄₋₁₅ alkenyl, wherein said C₁₋₆ alkyl and C₄₋₁₅ alkenyl are each optionally substituted with 1, 2, or 3 substituents independently selected from R^{g}; and
each R^{g} is independently selected from OH, C₁₋₆ alkyl, C₁₋₆ alkoxy, HO—C₁₋₃ alkylene, amino, C₁₋₆ alkylamino, and di(C₁₋₆ alkyl)amino.

In some embodiments:
R¹, R², R³, R⁴, R⁵, R⁶, R⁷, R⁸, R⁹, and R¹⁰ are each independently selected from H, Cy^{A}, C₁₋₆ alkyl, C₄₋₁₅ alkenyl, and OR^{a1}, wherein said C₁₋₆ alkyl and C₄₋₁₅ alkenyl are each optionally substituted with OR^{a1};
each R^{a1}, R^{b1}, R^{c1}, and R^{d1} is independently selected from H, Cy^{A}, C₁₋₆ alkyl, and C₄₋₁₅ alkenyl;
each Cy^{A} is independently selected from C₆₋₁₀ aryl, C₃₋₈ cycloalkyl, and 4-10 membered heterocycloalkyl, each of which is optionally substituted with 1, 2, or 3 substituents independently selected from R^{Cy};
each R^{Cy} is independently selected from C₁₋₆ alkyl, C₆₋₁₀ aryl, C₄₋₁₅ alkenyl, OR^{a2}, C(O)R^{b2}, and OC(O)R^{b2}, wherein said C₁₋₆ alkyl, C₄₋₁₅ alkenyl, and C₆₋₁₀ aryl are each optionally substituted by 1, 2, or 3 substituents independently selected from OR^{a2} and OC(O)R^{b2};
each R^{a2}, R^{b2}, R^{c2}, and R^{d2} is independently selected from H, C₁₋₆ alkyl, C₄₋₁₅ alkenyl, C₆₋₁₀ aryl, and 4-10 membered heterocycloalkyl, each of which is optionally substituted with 1, 2, or 3 substituents independently selected from R^{A};
each R^{A} is independently selected from C₁₋₆ alkyl, OR^{a3}, and OC(O)R^{b3}, wherein said C₁₋₆ alkyl is optionally substituted with 1, 2, or 3 substituents selected from OR^{a3} and OC(O)R^{b3};
each R^{a3}, R^{b3}, R^{c3}, and R^{d3} is independently selected from H, C₁₋₆ alkyl, and C₄₋₁₅ alkenyl, wherein said C₁₋₆ alkyl and C₄₋₁₅ alkenyl are each optionally substituted with 1, 2, or 3 substituents independently selected from R^{g}; and
each R^{g} is independently selected from OH, C₁₋₆ alkyl, C₁₋₆ alkoxy, HO—C₁₋₃ alkylene, amino, C₁₋₆ alkylamino, and di(C₁₋₆ alkyl)amino.

In some embodiments, the compound of Formula (I) is has formula:

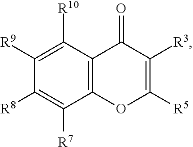

or a pharmaceutically acceptable salt thereof.

In some embodiments, the compound has formula:

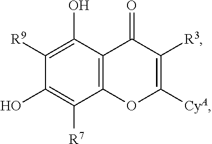

or a pharmaceutically acceptable salt thereof.

In some embodiments, the compound has formula:

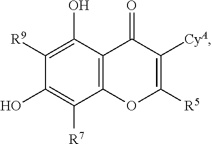

or a pharmaceutically acceptable salt thereof.

In some embodiments, the compound of Formula (I) has formula:

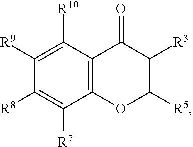

or a pharmaceutically acceptable salt thereof.

In some embodiments, the compound of Formula (I) has formula:

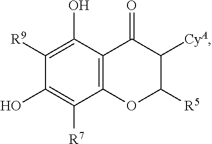

or a pharmaceutically acceptable salt thereof.

In some embodiments, the compound of Formula (I) has formula:

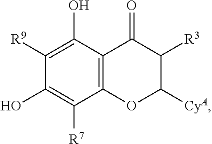

or a pharmaceutically acceptable salt thereof.

In some embodiments, the compound of Formula (I) has formula:

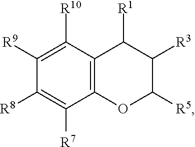

or a pharmaceutically acceptable salt thereof.

In some embodiments, the compound of Formula (I) has formula:

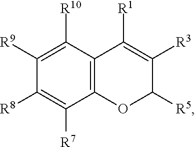

or a pharmaceutically acceptable salt thereof.

In some embodiments, the compound of Formula (I) comprises at least one C₄₋₁₅ alkenyl group selected from a moiety of any one of the following formulae:

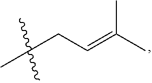 [SMILES: *CC=C(C)C]

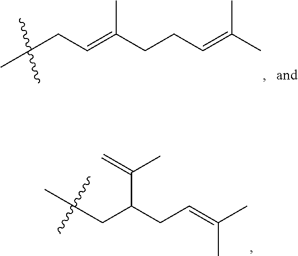 [SMILES: *C/C=C(\C)CCC=C(C)C ; *CC(CC=C(C)C)C(=C)C]

each of which is optionally substituted with 1, 2 or 3 substituents independently selected from OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, NR^{c1}S(O)₂NR^{c1}R^{d1}, S(O)₂R^{b1}, and S(O)₂NR^{c1}R^{d1}.

In some embodiments, the compound of Formula (I) is selected from any one of the compounds listed in any one of Tables 1a-2e, or a pharmaceutically acceptable salt thereof.

In a general aspect, the present disclosure provides a method of treating or preventing a viral infection caused by a coronavirus selected from SARS-CoV, SAR-CoV-2, and MERS-CoV in a subject, the method comprising administering to the subject a therapeutically effective amount of a compound of Formula (I) as described herein, or a pharmaceutically acceptable salt thereof.

In some embodiments, the viral infection is coronavirus disease 2019 (COVID-19).

In some embodiments, the viral infection is severe acute respiratory syndrome (SARS).

In some embodiments, the viral infection is Middle East respiratory syndrome (MERS).

In some embodiments, the method further comprises administering to the subject a therapeutically effective amount of an antiviral agent, or a pharmaceutically acceptable salt thereof.

In a general aspect, the present disclosure provides a method of treating or preventing a viral infection caused by a coronavirus selected from SARS-CoV, SAR-CoV-2, and MERS-CoV in a subject, the method comprising intranasally administering to the subject in need thereof a therapeutically effective amount of a compound of Formula (I):

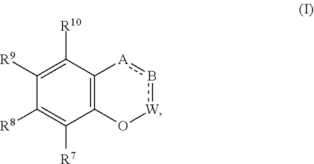

or a pharmaceutically acceptable salt thereof, wherein:
A is CR¹R²;
B is CR³R⁴;
W is CR⁵R⁶.
R¹, R², R³, R⁴, R⁵, R⁶, R⁷, R⁸, R⁹, and R¹⁰ are each independently selected from H, Cy^{A}, halo, CN, NO₂, C₁₋₆ alkyl, C₁₋₆ haloalkyl, C₄₋₁₅ alkenyl, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl and C₄₋₁₅ alkenyl are each optionally substituted with 1, 2 or 3 substituents independently selected from Cy^{A}, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1} and S(O)₂NR^{c1}R^{d1};
each [symbol] bond is either a single bond or a double bond, provided that:
(iii) when the bond between A and B is a double bond, then R² and R⁴ are absent and the bond between B and W is a single bond; and
(iv) when the bond between B and W is a double bond, then R⁴ and R⁶ are absent and the bond between A and B is a single bond;
or R¹ and R² together form an oxo group;
or R³ and R⁵, together with the carbon atoms to which they are attached, form 4-10 membered heterocycloalkyl, which is optionally substituted with 1, 2, 3, 4, or 5 substituents independently selected from R^{Cy};
or R¹ and R³, together with the carbon atoms to which they are attached, form 4-10 membered heterocycloalkyl, which is optionally substituted with 1, 2, 3, 4, or 5 substituents independently selected from R^{Cy};
R⁷ and R⁸, together with the carbon atoms to which they are attached, form a 4-10 membered heterocycloalkyl, which is optionally substituted with 1, 2, 3, or 4 substituents independently selected from R^{Cy};
R⁸ and R⁹, together with the carbon atoms to which they are attached, form a 4-10 membered heterocycloalkyl, which is optionally substituted with 1, 2, 3, or 4 substituents independently selected from R^{Cy};
R⁹ and R¹⁰, together with the carbon atoms to which they are attached, form a 4-10 membered heterocycloalkyl, which is optionally substituted with 1, 2, 3, or 4 substituents independently selected from R^{Cy};
each R^{a1}, R^{b1}, R^{c1}, and R^{d1} is independently selected from H, Cy^{A}, C₁₋₆ alkyl, C₄₋₁₅ alkenyl, C₁₋₆ haloalkyl, wherein said C₁₋₆ alkyl and C₄₋₁₅ alkenyl are each optionally substituted with 1, 2, or 3 substituents independently selected from R^{g}; each Cy^{A} is independently selected from C₆₋₁₀ aryl, C₃₋₈ cycloalkyl, and 4-10 membered heterocycloalkyl, each of which is optionally substituted with 1, 2, 3, 4, or 5 substituents independently selected from R^{Cy};
each R^{Cy} is independently selected from halo, C₁₋₆ alkyl, C₁₋₆ haloalkyl, C₆₋₁₀ aryl, C₄₋₁₅ alkenyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}, wherein said C₁₋₆ alkyl, C₄₋₁₅ alkenyl, and C₆₋₁₀ aryl are each optionally substituted by 1, 2, or 3 substituents independently selected from C₁₋₆ alkyl, C₁₋₆ haloalkyl, halo, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
each R^{a2}, R^{b2}, R^{c2}, and R^{d2} is independently selected from H, C₁₋₆ alkyl, C₄₋₁₅ alkenyl, C₆₋₁₀ aryl, and 4-10 membered heterocycloalkyl, each of which is optionally substituted with 1, 2, 3, 4, or 5 substituents independently selected from R^{A};
each R^{A} is independently selected from C₁₋₆ alkyl, C₁₋₆haloalkyl, halo, CN, NO₂, OR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}, wherein said C₁₋₆ alkyl is optionally substituted with 1, 2, or 3 substituents selected from CN, NO₂, OR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3};
each R^{a3}, R^{b3}, R^{c3}, and R^{d3} is independently selected from H, C₁₋₆ alkyl, C₄₋₁₅ alkenyl, C₁₋₆ haloalkyl, wherein said C₁₋₆ alkyl and C₄₋₁₅ alkenyl are each optionally substituted with 1, 2, or 3 substituents independently selected from R^{g};
or any R^{c1} and R^{d1} together with the N atom to which they are attached form a 4-, 5-, 6-, or 7-membered heterocycloalkyl group optionally substituted with 1, 2, or 3 substituents independently selected from R^{g};
or any R^{c2} and R^{d2} together with the N atom to which they are attached form a 4-, 5-, 6-, or 7-membered heterocycloalkyl group optionally substituted with 1, 2, or 3 substituents independently selected from R^{g};
or any R^{c3} and R^{d3} together with the N atom to which they are attached form a 4-, 5-, 6-, or 7-membered heterocycloalkyl group optionally substituted with 1, 2, or 3 substituents independently selected from R^{g}; and
each R^{g} is independently selected from OH, NO₂, CN, halo, C₁₋₆ alkyl, C₁₋₄ haloalkyl, C₁₋₆ alkoxy, C₁₋₆ haloalkoxy, cyano-C₁₋₃ alkylene, HO—C₁₋₃ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

In some embodiments, the compound of Formula (I) is selected from quercetin, luteolin, baicalin, amentoflavone, hesperetin, hesperidin, hervacetin, rhoifolin, pectolinarin, gallocatechin, gallocatechin gallate, diosmin, kaempferol, luteolin-7-glucoside, quercetin-3-β-galactoside, naringenin, apigenin-7-glucoside, catechin, pigallocatechin, isoquercetin, epigallocatechin, herbacetin, isotheaflavin, baicalein, theacitrin A, corilagin, theaflavin, (-)-taxifolin, Rhamnetin, quercetin 3-glucuronide-7-glucoside, quercetin 3-vicianoside, delphinidin 3-O-glucoside, petunidin 3-O-glucoside, chrysoeriol 8-C-glucoside, schaftoside, rutin, hypericin, cyanidin 3-glucoside, glabridin, orientin, astilbin, puerarin, astragalin, 2′-hydroxygenistein, genistein, morin, dihydromorin, steppogenin, 6-methoxyluteolin, pinocembrin 7-O-(3″-galloyl-4″,6″-(S)-hexahydroxydiphenoyl)-β-D-glucose (PGHG), biorobin, jaceidin triacetate, kaempferol-4′-O-beta-D-glucopyranoside, dracoflavan B1, isoschaftoside, norartocarpetin, 3′,4′,5,7-tetra hydroxyisoflavanone, and artocarpanone, or a pharmaceutically acceptable salt thereof.

In some embodiments, the compound of Formula (I) is selected from pinocembrin 7-O-(3″-galloyl-4″,6″-(S)-hexahydroxydiphenoyl)-β-D-glucose (PGHG), Amentoflavone, Jaceidin triacetate, 3,8′-Biapigenin, Baicalein, myricetin, apigenin, bilobetin, Ginkgetin, Isoginkgetin, Podocarpus flavone A, 2,3-dihydro-heveaflavone, Tetrahydro-amentoflavone, Apigenin 6-C-alpha-L-arabinopyranosyl-8-C-beta-D-xylopyranoside, 3,6-Dimethoxyapigenin, Apigenin 7-glucuronide, 7,4′-Di-O-methylapigenin 5-O-xylosylglucoside, Apigenin 4′-O-rhamnoside, Apigenin 5-O-neohesperidoside, 7,4′-Di-O-methylapigenin, and Pinocembrin.

In some embodiments, the viral infection is coronavirus disease 2019 (COVID-19).

In some embodiments, the viral infection is severe acute respiratory syndrome (SARS).

In some embodiments, the viral infection is Middle East respiratory syndrome (MERS).

In some embodiments, the method further comprises administering to the subject a therapeutically effective amount of an antiviral agent, or a pharmaceutically acceptable salt thereof.

Unless otherwise defined, all technical and scientific terms used herein have the same meaning as commonly understood by one of ordinary skill in the art to which the present application belongs. Methods and materials are described herein for use in the present application; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

In some embodiments, the present disclosure provides a method of inhibiting protein disulfide isomerase (PDI) in a cell, the method comprising contacting the cell with an effective amount of a compound of Formula (I) as described herein, or a pharmaceutically acceptable salt thereof.

In some embodiments, the present disclosure provides a method of inhibiting protein disulfide isomerase (PDI) in a subject, the method comprising administering to the subject a therapeutically effective amount of a compound of Formula (I) as described herein, or a pharmaceutically acceptable salt thereof,

In some embodiments, the present disclosure provides a method of inhibiting thrombosis in a subject, the method comprising administering to the subject a therapeutically effective amount of a compound of Formula (I) as disclosed herein, or a pharmaceutically acceptable salt thereof,

Other features and advantages of the present application will be apparent from the following detailed description and figures, and from the claims.

### DESCRIPTION OF DRAWINGS

FIG. 1 contains a crystal structure of SARS-CoV-2 M^{pro} protein. Two views of the three-dimensional crystal structure of SARS-CoV-2 M^{pro} are shown (adapted from Zhang et al., Science, 368: 409 (FIG. 2); DOI: 10.1126/science.abb3405).

FIG. 2A contains a consensus sequence of SARS-CoV M^{pro} cleavage. The 11 sequences within polyprotein 1a (pp1a) and polyprotein 1ab (pp1ab) of SARS-CoV-2 that are cleaved by SARS-CoV 3CL^{pro} (also known as SARS-CoV main protease) (adapted from Muramatsu et al., PNAS, 113: 12997 (FIG. 1D); doi.org/10.1073/pnas.1601327113).

FIG. 2B contains a consensus sequence of SARS-CoV M^{pro} cleavage. The figure shows consensus sequence for cleavage by 3CL^{pro}. (adapted from Muramatsu et al., PNAS, 113: 12997 (FIG. 1E); doi.org/10.1073/pnas.1601327113).

FIG. 3A contains a construct for generating SARS-CoV-2 M^{pro}.

FIG. 3B contains a schematic representation of a fluorescence-based assay to detect activity SARS-CoV-2 M^{pro} enzyme (adapted from Kasperkeiwicz et al., FEBS J., 284:1518 (FIG. 4); doi:10.1111/febs.14001).

FIG. 4A shows Evaluation of flavonoid Mpro inhibitors in a mass spectroscopy-based assay.

FIG. 4B shows Characteristics of exemplified tested compounds.

FIG. 5A shows Comparison of IC_{50S} of amentoflavone, 3,8′-biapigenin, bilobetin, and ginkgetin as inhibitors of Mpro.

FIG. 5B shows binding pose of amentoflavone (structure shown as sticks; oxygen; nitrogen) in the catalytic domain of Mpro (shown as surface; hydrophilic; hydrophobic).

FIG. 5C shows H-bond interactions (shown as blue dashed line) between amentoflavone (structure shown in cyan as sticks) and Mpro (backbone shown in blue as ribbon/helix/coil, and sidechain shown as wire; red: oxygen; blue: nitrogen, yellow: sulfur).

FIG. 6A shows Comparison of IC_{50S} of PGHG, jaceidin acetate, baicalein, and myricetin.

FIG. 6B shows Evaluation of reversibility of PGHG, apigenin, amentoflavone, 3,8′-biapigenin, baicalein, and myricetin.

FIG. 7A shows Dose curve of inhibition of SARS-CoV-2 replication in Vero E6 cells by PGHG, apigenin, amentoflavone, 3,8′-biapigenin, baicalein, and myricetin.

FIG. 7B shows Images of inhibition of viral replication by PGHG, apigenin, amentoflavone, 3,8′-biapigenin, baicalein, and myricetin.

FIG. 8 shows Activity of PGHG in the SARS-CoV-2 replication assay.

FIG. 9 Mass spectroscopy assay to detect inhibition of Mpro. The substrate peptide KTSAVLQSGFRKM was incubated with Mpro for 2 hours prior to analysis by LC/MS. Tracings for the full length (top panel), the N-terminal product (KTSAVLQS; middle panel), and the C-terminal product (GFRKM; bottom panel) are shown.

FIG. 10 shows inhibitory activity by selected flavonoids.

FIG. 11 shows inhibitory activity by selected apigenin analogs

FIG. 12 shows inhibitory activity of pinocembrin.

FIG. 13 shows chemical structures and IC_{50S} of selected flavonoids.

FIG. 14 shows thrombus formation following laser-induced injury of a cremaster arteriole. Image shows platelet formation and fibrin formation.

### DETAILED DESCRIPTION

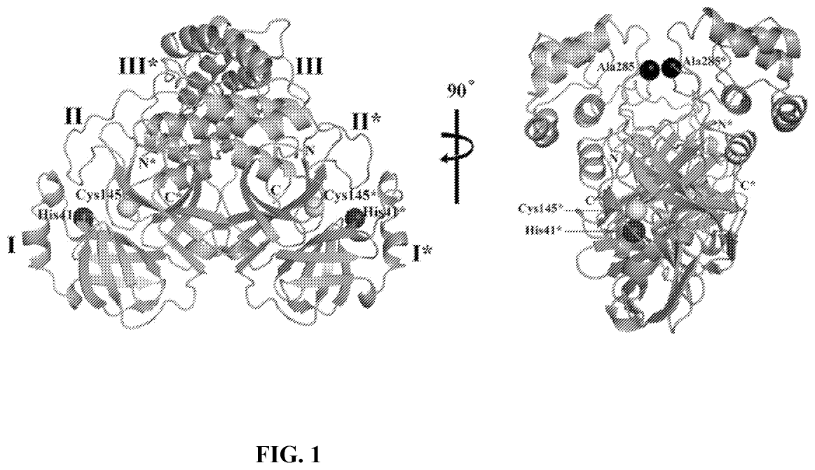

SARS-CoV-2, the virus responsible for Covid-19, encodes two polypeptides that contain proteins responsible for replicase activity. Polyprotein 1ab includes two proteases, papain-like (PL) proteinase and 3-chymotrypsin-like (3CL) protease, as well as several other proteins involved in viral replication. 3CL protease is also known as the SARS-CoV-2 main protease (SARS-CoV-2 M^{pro}) because it cleaves the polyprotein at 11 different cleavage sites to generate the majority of proteins encoded in polyprotein 1ab. SARS-CoV-2 M^{pro} is a cysteine-protease with an active site cysteine at C145 and a catalytic histidine at H41. The X-ray crystallography-derived structure of SARS-CoV-2 M^{pro} was recently solved (See Zhang et al, Science, 2020; 3405(March):1-9. doi:10.1126/science.abb3405). The protein consists of three domains. Domains I (10-99) and II (100-182) are adjacent β-sheets that harbor a substrate-binding site in a cleft between them. Domain III (198-303) consists of 5 u-helices that regulate dimerization (See FIG. 1, adapted from Zhang et al., Science, 36 (6489), 409-12), which is thought to affect the enzyme's activity.

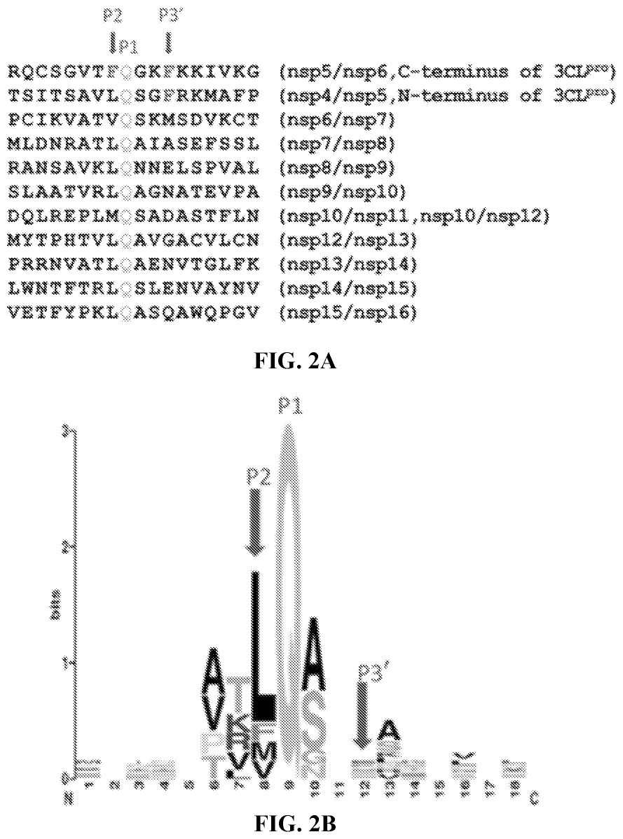

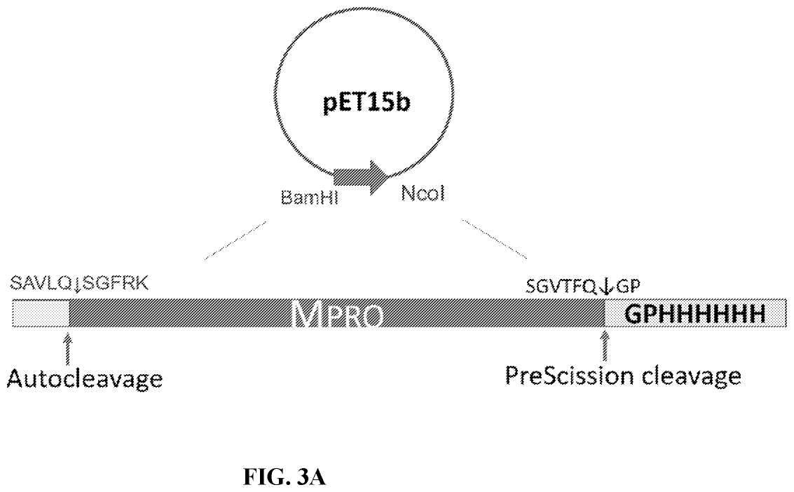

The SARS-CoV-2 RNA genome is about 82% similar to the SARS-CoV RNA genome and Main Proteases from the two viruses share about 96% sequence identity (for consequence sequence of SARS-CoV main protease, see FIGS. 2A and 2B, adapted from Muramatsu et al., PNAS, 113 (46), 12997-13002). It is believed that the residues involved in the catalysis, substrate recognition, and dimerization are 100% conserved (See Chen et al, F1000Research. 2020(9), 129; doi: 10.12688/f1000research.22457.2; see also Yang et al., Proc Natl Acad Sci USA. 2003; 100(23):13190-13195. doi:10.1073/pnas.1835675100). SARS-CoV M^{pro} cleaves at glutamine with a consensus sequence of Leu-Gln-Ser/Ala as the preferred P2-P1-P1′ and SARS-CoV-2 M^{pro} has a similar consensus sequence (See Muramatsu et al., PNAS, 113 (46), 12997-13002; doi:10.1073/pnas.1601327113). Without being bound by a theory, it is believed that the substrate binding site consists of six subsites (S1-S6), which correspond to the P1-P6 peptides in the substrate polyprotein. The conserved Gln of the polypeptide lies in the S1 subsite (See Yang et al., Proc Natl Acad Sci USA. 2003; 100(23):13190-13195. doi:10.1073/pnas.1835675100; see also Li et al., Sci Rep. 2016; 6:20918. doi:10.1038/srep20918). At the catalytic dyad, His41 facilitates the deprotonation of the thiol of Cys145. This allows for nucleophilic attack of Gln within the S1 subsite and release of the N-terminus. This is followed by hydrolysis of the resulting thioester. A short helix Ser139-Leu141 disrupts the catalytic machinery in the monomer, but is displaced upon dimer formation (Li et al., Sci Rep. 2016; 6:20918. doi:10.1038/srep20918).

Accordingly, the present disclosure provides flavonoid compounds that inhibit SARS-CoV-2 main protease, as well as other coronaviral proteases. Methods of using these compounds for treating viral infections, pharmaceutical compositions containing these compounds, and combination treatments are also described in the present disclosure.

Therapeutic Compounds

Flavonoids are a class of naturally occurring compounds this is rich in structural variety and pharmacological activity. Prenylated flavonoids are a subclass of flavonoid compounds that is often found in plants, for example, in the Leguminosae, Fabaceae, and Moraceae plant families. In some embodiments, the present disclosure provides a flavonoid compound, or a pharmaceutically acceptable salt thereof. In other embodiments, the present disclosure provides a prenylated flavonoid compound, or a pharmaceutically acceptable salt thereof. In these embodiments, the disclosure provides a plant extract containing at least one prenylated flavonoid compound. Examples of such extracts include a dry extract, an aqueous extract, and an alcoholic extract (e.g., ethanolic, methanolic, hydroethanolic, or hydromehtanolic extract). Examples of plants from which prenylated and non-prenylated flavonoid compounds of the disclosure may be obtained (e.g., extracted) include Morus alba L. (white mulberry), Morus nigra L. (black mulberry), Morus rubra, Humulus lupulus (common hop), Glycine max (soybean), Maackia amurensis, Artocarpus heterophyllus (jackfruit), Artocarpus rigida (monkey jackfruit), Artocarpus altilis (breadfruit), Artocarpus dadah, and Artocarpus elasticus, as well and other plant species. In alternative embodiments, flavonoid compounds of this disclosure may be prepared using chemical synthesis techniques. For example, a prenylated flavonoid may be prepared by introducing a prenyl group (or a related alkenyl group such as a geranyl group or a lavandulyl group) into the chemical structure of a non-prenylated natural flavonoid. In such an example, a flavonoid compound containing a hydroxyl group may be reacted with a suitable electrophile, such as a prenyl bromide.

In some embodiments, the present disclosure provides a compound of Formula (I):

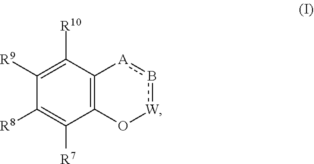

or a pharmaceutically acceptable salt thereof, wherein:
A is CR¹R²;
B is CR³R⁴;
W is CR⁵R⁶;
R¹, R², R³, R⁴, R⁵, R⁶, R⁷, R⁸, R⁹, and R¹⁰ are each independently selected from H, Cy^{A}, halo, CN, NO₂, C₁₋₆ alkyl, C₁₋₆ haloalkyl, C₄₋₁₅ alkenyl, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl and C₄₋₁₅ alkenyl are each optionally substituted with 1, 2 or 3 substituents independently selected from Cy^{A}, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1} and S(O)₂NR^{c1}R^{d1};
each [symbol] bond is either a single bond or a double bond, provided that:
(i) when the bond between A and B is a double bond, then R² and R⁴ are absent and the bond between B and W is a single bond; and
(ii) when the bond between B and W is a double bond, then R⁴ and R⁶ are absent and the bond between A and B is a single bond;
or R¹ and R² together form an oxo group;
or R³ and R⁵, together with the carbon atoms to which they are attached, form 4-10 membered heterocycloalkyl, which is optionally substituted with 1, 2, 3, 4, or 5 substituents independently selected from R^{Cy};
or R¹ and R³, together with the carbon atoms to which they are attached, form 4-10 membered heterocycloalkyl, which is optionally substituted with 1, 2, 3, 4, or 5 substituents independently selected from R^{Cy};
R⁷ and R⁸, together with the carbon atoms to which they are attached, form a 4-10 membered heterocycloalkyl, which is optionally substituted with 1, 2, 3, or 4 substituents independently selected from R^{Cy};
R⁸ and R⁹, together with the carbon atoms to which they are attached, form a 4-10 membered heterocycloalkyl, which is optionally substituted with 1, 2, 3, or 4 substituents independently selected from R^{Cy};
R⁹ and R¹⁰, together with the carbon atoms to which they are attached, form a 4-10 membered heterocycloalkyl, which is optionally substituted with 1, 2, 3, or 4 substituents independently selected from R^{Cy};
each R^{a1}, R^{b1}, R^{c1}, and R^{d1} is independently selected from H, Cy^{A}, C₁₋₆ alkyl, C₄₋₁₅ alkenyl, C₁₋₆ haloalkyl, wherein said C₁₋₆ alkyl and C₄₋₁₅ alkenyl are each optionally substituted with 1, 2, or 3 substituents independently selected from R^{g};
each Cy^{A} is independently selected from C₆₋₁₀ aryl, C₃₋₈ cycloalkyl, and 4-10 membered heterocycloalkyl, each of which is optionally substituted with 1, 2, 3, 4, or 5 substituents independently selected from R^{Cy};
each R^{Cy} is independently selected from halo, C₁₋₆ alkyl, C₁₋₆ haloalkyl, C₆₋₁₀ aryl, C₄₋₁₅ alkenyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}, wherein said C₁₋₆ alkyl, C₄₋₁₅ alkenyl, and C₆₋₁₀ aryl are each optionally substituted by 1, 2, or 3 substituents independently selected from C₁₋₆ alkyl, C₁₋₆ haloalkyl, halo, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
each R^{a2}, R^{b2}, R^{c2}, and R^{d2} is independently selected from H, C₁₋₆ alkyl, C₄₋₁₅ alkenyl, C₆₋₁₀ aryl, and 4-10 membered heterocycloalkyl, each of which is optionally substituted with 1, 2, 3, 4, or 5 substituents independently selected from R^{A};
each R^{A} is independently selected from C₁₋₆ alkyl, C₁₋₆haloalkyl, halo, CN, NO₂, OR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}, wherein said C₁₋₆ alkyl is optionally substituted with 1, 2, or 3 substituents selected from CN, NO₂, OR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3};
each R^{a3}, R^{b3}, R^{c3}, and R^{d3} is independently selected from H, C₁₋₆ alkyl, C₄₋₁₅ alkenyl, C₁₋₆ haloalkyl, wherein said C₁₋₆ alkyl and C₄₋₁₅ alkenyl are each optionally substituted with 1, 2, or 3 substituents independently selected from R^{g};
or any R^{c1} and R^{d1} together with the N atom to which they are attached form a 4-, 5-, 6-, or 7-membered heterocycloalkyl group optionally substituted with 1, 2, or 3 substituents independently selected from R^{g};
or any R^{c2} and R^{d2} together with the N atom to which they are attached form a 4-, 5-, 6-, or 7-membered heterocycloalkyl group optionally substituted with 1, 2, or 3 substituents independently selected from R^{g};
or any R^{c3} and R^{d3} together with the N atom to which they are attached form a 4-, 5-, 6-, or 7-membered heterocycloalkyl group optionally substituted with 1, 2, or 3 substituents independently selected from R^{g}; and
each R^{g} is independently selected from OH, NO₂, CN, halo, C₁₋₆ alkyl, C₁₋₄ haloalkyl, C₁₋₆ alkoxy, C₁₋₆ haloalkoxy, cyano-C₁₋₃ alkylene, HO—C₁₋₃ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

In some embodiments, R¹, R², R³, R⁴, R⁵, R⁶, R⁷, R⁸, R⁹, and R¹⁰ are each independently selected from H, Cy^{A}, halo, C₁₋₆ alkyl, C₁₋₆ haloalkyl, C₄₋₁₅ alkenyl, OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, and NR^{c1}S(O)₂NR^{c1}R^{d1}; wherein said C₁₋₆ alkyl and C₄₋₁₅ alkenyl are each optionally substituted with 1, 2 or 3 substituents independently selected from Cy^{A}, halo, OR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, NR^{c1}S(O)₂NR^{c1}R^{d1}, S(O)₂R^{b1}, and S(O)₂NR^{c1}R^{d1}.

In some embodiments, R¹, R², R³, R⁴, R⁵, R⁶, R⁷, R⁸, R⁹, and R¹⁰ are each independently selected from H, Cy^{A}, C₁₋₆ alkyl, C₄₋₁₅ alkenyl, and OR^{a1}, wherein said C₁₋₆ alkyl and C₄₋₁₅ alkenyl are each optionally substituted with OR^{a1}.

In some embodiments, at least one of R¹, R², R³, R⁴, R⁵, R⁶, R⁷, R⁸, R⁹, and R¹⁰ is C₄₋₁₅ alkenyl (e.g., a prenyl group described herein), optionally substituted with 1, 2, or 3 substituents independently selected from Cy^{A}, halo, OR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, NR^{c1}S(O)₂NR^{c1}R^{d1}, S(O)₂R^{b1}, and S(O)₂NR^{c1}R^{d1}.

In some embodiments, at least one of R¹, R², R³, R⁴, R⁵, R⁶, R⁷, R⁸, R⁹, and R¹⁰ is C₄₋₁₅ alkenyl (e.g., a prenyl group described herein), optionally substituted with OR^{a1}. In some embodiments, at least one of R¹, R², R³, R⁴, R⁵, R⁶, R⁷, R⁸, R⁹, and R¹⁰ is C₄₋₁₅ alkenyl (e.g., a prenyl group described herein).

In some embodiments, at least one of R¹, R², R³, R⁴, R⁵, R⁶, R⁷, R⁸, R⁹, and R¹⁰ is OR^{a1}. In some embodiments, at least one of R¹, R², R³, R⁴, R⁵, R⁶, R⁷, R⁸, R⁹, and R¹⁰ is Cy^{A}.

In some embodiments, each R^{a1}, R^{b1}, R^{c1}, and R^{d1} is independently selected from H, Cy^{A}, C₁₋₆ alkyl, and C₄₋₁₅ alkenyl.

In some embodiments, R^{a1} is C₄₋₁₅ alkenyl (e.g., a prenyl group described herein), optionally substituted with 1, 2, or 3 substituents independently selected from Cy^{A}, halo, OR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, NR^{c1}S(O)₂NR^{c1}R^{d1}, S(O)₂R^{b1}, and S(O)₂NR^{c1}R^{d1}.

In some embodiments, R^{a1} is C₄₋₁₅ alkenyl (e.g., a prenyl group described herein), optionally substituted with OR^{a1}.

In some embodiments, R^{a1} is C₄₋₁₅ alkenyl (e.g., a prenyl group described herein).

In some embodiments, R^{a1} is Cy^{A}. In some embodiments, R^{a1} is C₁₋₆ alkyl.

In some embodiments, Cy^{A} is selected from C₆₋₁₀ aryl, C₃₋₈ cycloalkyl, and 4-10 membered heterocycloalkyl, each of which is optionally substituted with 1, 2, or 3 substituents independently selected from R^{Cy}.

In some embodiments, Cy^{A} is C₆₋₁₀ aryl, optionally substituted with 1, 2, or 3 substituents independently selected from R^{Cy}.

In some embodiments, Cy^{A} is C₃₋₈ cycloalkyl, optionally substituted with 1, 2, or 3 substituents independently selected from R^{Cy}.

In some embodiments, Cy^{A} is 4-10 membered heterocycloalkyl, optionally substituted with 1, 2, or 3 substituents independently selected from R^{Cy}.

In some embodiments, R^{Cy} is selected from halo, C₁₋₆ alkyl, C₆₋₁₀ aryl, C₄₋₁₅ alkenyl, OR^{a2}, C(O)R^{b2}, OC(O)R^{b2}, OC(O)NR^{c2}R^{d2}, NR^{c2}R^{d2}, NR^{c2}C(O)R^{b2}, NR^{c2}C(O)OR^{a2}, and NR^{c2}C(O)NR^{c2}R^{d2}, wherein said C₁₋₆ alkyl, C₄₋₁₅ alkenyl, and C₆₋₁₀ aryl are each optionally substituted by 1, 2, or 3 substituents independently selected from C₁₋₆ alkyl, OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, and NR^{c2}C(O)NR^{c2}R^{d2}.

In some embodiments, R^{Cy} is selected from C₁₋₆ alkyl, C₆₋₁₀ aryl, C₄₋₁₅ alkenyl, OR^{a2}, C(O)R^{b2}, and OC(O)R^{b2}, wherein said C₁₋₆ alkyl, C₄₋₁₅ alkenyl, and C₆₋₁₀ aryl are each optionally substituted by 1, 2, or 3 substituents independently selected from OR^{a2} and OC(O)R^{b2}.

In some embodiments, R^{Cy} is C₄₋₁₅ alkenyl (e.g., a prenyl group described herein), optionally substituted with 1, 2, or 3 substituents independently selected from C₁₋₆ haloalkyl, halo, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}.

In some embodiments, R^{Cy} is C₄₋₁₅ alkenyl (e.g., a prenyl group described herein), optionally substituted with OR^{a2}. In some embodiments, R^{Cy} is C₄₋₁₅ alkenyl (e.g., a prenyl group described herein).

In some embodiments, R^{Cy} is C₆₋₁₀ aryl, optionally substituted by 1, 2, or 3 substituents independently selected from C₁₋₆ alkyl, OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, and NR^{c2}C(O)NR^{c2}R^{d2}.

In some embodiments, R^{Cy} is C₁₋₆ alkyl, optionally substituted with OR^{a2} or OC(O)R^{b2}. In some embodiments, R^{Cy} is OR^{a2}. In some embodiments, R^{Cy} is selected from C(O)R^{b2} and OC(O)R^{b2}.

In some embodiments, each R^{a2}, R^{b2}, R^{c2}, and R^{d2} is independently selected from H, C₁₋₆ alkyl, C₄₋₁₅ alkenyl, C₆₋₁₀ aryl, and 4-10 membered heterocycloalkyl, each of which is optionally substituted with 1, 2, or 3 substituents independently selected from R^{A}.

In some embodiments, R^{a2} is C₁₋₆ alkyl. In some embodiments, R^{a2} is C₄₋₁₅ alkenyl, optionally substituted with 1, 2, or 3 substituents independently selected from R^{A}.

In some embodiments, R^{a2} is selected from C₆₋₁₀ aryl and 4-10 membered heterocycloalkyl, each of which is optionally substituted with 1, 2, or 3 substituents independently selected from R^{A}.

In some embodiments, each R^{A} is independently selected from C₁₋₆ alkyl, OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, and NR^{c3}C(O)NR^{c3}R^{d3}, wherein said C₁₋₆ alkyl is optionally substituted with 1, 2, or 3 substituents selected from OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, and NR^{c3}C(O)NR^{c3}R^{d3}.

In some embodiments, each R^{A} is independently selected from C₁₋₆ alkyl, OR^{a3}, and OC(O)R^{b3}, wherein said C₁₋₆ alkyl is optionally substituted with 1, 2, or 3 substituents selected from OR^{a3} and OC(O)R^{b3}.

In some embodiments, each R^{a3}, R^{b3}, R^{c3}, and R^{d3} is independently selected from H, C₁₋₆ alkyl, and C₄₋₁₅ alkenyl, wherein said C₁₋₆ alkyl and C₄₋₁₅ alkenyl are each optionally substituted with 1, 2, or 3 substituents independently selected from R^{g}.

In some embodiments, each R^{g} is independently selected from OH, C₁₋₆ alkyl, C₁₋₆ alkoxy, HO—C₁₋₃ alkylene, amino, C₁₋₆ alkylamino, and di(C₁₋₆ alkyl)amino.

In some embodiments of the compounds of Formula (I):
R¹, R², R³, R⁴, R⁵, R⁶, R⁷, R⁸, R⁹, and R¹⁰ are each independently selected from H, Cy^{A}, halo, C₁₋₆ alkyl, C₁₋₆ haloalkyl, C₄₋₁₅ alkenyl, OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, and NR^{c1}S(O)₂NR^{c1}R^{d1}; wherein said C₁₋₆ alkyl and C₄₋₁₅ alkenyl are each optionally substituted with 1, 2 or 3 substituents independently selected from Cy^{A}, halo, OR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, NR^{c1}S(O)₂NR^{c1}R^{d1}, S(O)₂R^{b1}, and S(O)₂NR^{c1}R^{d1};
each R^{a1}, R^{b1}, R^{c1}, and R^{d1} is independently selected from H, Cy^{A}, C₁₋₆ alkyl, and C₄₋₁₅ alkenyl;
each Cy^{A} is independently selected from C₆₋₁₀ aryl, C₃₋₈ cycloalkyl, and 4-10 membered heterocycloalkyl, each of which is optionally substituted with 1, 2, or 3 substituents independently selected from R^{Cy};
each R^{Cy} is independently selected from halo, C₁₋₆ alkyl, C₆₋₁₀ aryl, C₄₋₁₅ alkenyl, OR^{a2}, C(O)R^{b2}, OC(O)R^{b2}, OC(O)NR^{c2}R^{d2}, NR^{c2}R^{d2}, NR^{c2}C(O)R^{b2}, NR^{c2}C(O)OR^{a2}, and NR^{c2}C(O)NR^{c2}R^{d2}, wherein said C₁₋₆ alkyl, C₄₋₁₅ alkenyl, and C₆₋₁₀ aryl are each optionally substituted by 1, 2, or 3 substituents independently selected from C₁₋₆ alkyl, OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, and NR^{c2}C(O)NR^{c2}R^{d2};
each R^{a2}, R^{b2}, R^{c2}, and R^{d2} is independently selected from H, C₁₋₆ alkyl, C₄₋₁₅ alkenyl, C₆₋₁₀ aryl, and 4-10 membered heterocycloalkyl, each of which is optionally substituted with 1, 2, or 3 substituents independently selected from R^{A};
each R^{A} is independently selected from C₁₋₆ alkyl, OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, and NR^{c3}C(O)NR^{c3}R^{d3}, wherein said C₁₋₆ alkyl is optionally substituted with 1, 2, or 3 substituents selected from OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, and NR^{c3}C(O)NR^{c3}R^{d3};
each R^{a3}, R^{b3}, R^{c3}, and R^{d3} is independently selected from H, C₁₋₆ alkyl, and C₄₋₁₅ alkenyl, wherein said C₁₋₆ alkyl and C₄₋₁₅ alkenyl are each optionally substituted with 1, 2, or 3 substituents independently selected from R^{g}; and
each R^{g} is independently selected from OH, C₁₋₆ alkyl, C₁₋₆ alkoxy, HO—C₁₋₃ alkylene, amino, C₁₋₆ alkylamino, and di(C₁₋₆ alkyl)amino.

In some embodiments of the compound of Formula (I):
R¹, R², R³, R⁴, R⁵, R⁶, R⁷, R⁸, R⁹, and R¹⁰ are each independently selected from H, Cy^{A}, C₁₋₆ alkyl, C₄₋₁₅ alkenyl, and OR^{a1}, wherein said C₁₋₆ alkyl and C₄₋₁₅ alkenyl are each optionally substituted with OR^{a1};
each R^{a1}, R^{b1}, R^{c1}, and R^{d1} is independently selected from H, Cy^{A}, C₁₋₆ alkyl, and C₄₋₁₅ alkenyl;
each Cy^{A} is independently selected from C₆₋₁₀ aryl, C₃₋₈ cycloalkyl, and 4-10 membered heterocycloalkyl, each of which is optionally substituted with 1, 2, or 3 substituents independently selected from R^{Cy};
each R^{Cy} is independently selected from C₁₋₆ alkyl, C₆₋₁₀ aryl, C₄₋₁₅ alkenyl, OR^{a2}, C(O)R^{b2}, and OC(O)R^{b2}, wherein said C₁₋₆ alkyl, C₄₋₁₅ alkenyl, and C₆₋₁₀ aryl are each optionally substituted by 1, 2, or 3 substituents independently selected from OR^{a2} and OC(O)R^{b2};
each R^{a2}, R^{b2}, R^{c2}, and R^{d2} is independently selected from H, C₁₋₆ alkyl, C₄₋₁₅ alkenyl, C₆₋₁₀ aryl, and 4-10 membered heterocycloalkyl, each of which is optionally substituted with 1, 2, or 3 substituents independently selected from R^{A};
each R^{A} is independently selected from C₁₋₆ alkyl, OR^{a3}, and OC(O)R^{b3}, wherein said C₁₋₆ alkyl is optionally substituted with 1, 2, or 3 substituents selected from OR^{a3} and OC(O)R^{b3};
each R^{a3}, R^{b3}, R^{c3}, and R^{d3} is independently selected from H, C₁₋₆ alkyl, and C₄₋₁₅ alkenyl, wherein said C₁₋₆ alkyl and C₄₋₁₅ alkenyl are each optionally substituted with 1, 2, or 3 substituents independently selected from R^{g}; and
each R^{g} is independently selected from OH, C₁₋₆ alkyl, C₁₋₆ alkoxy, HO—C₁₋₃ alkylene, amino, C₁₋₆ alkylamino, and di(C₁₋₆ alkyl)amino.

In some embodiments, the compound of Formula (I) has formula:

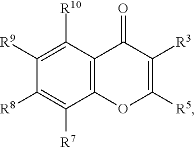

or a pharmaceutically acceptable salt thereof.

In some embodiments, the compound of Formula (I) has formula:

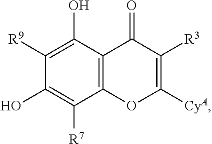

or a pharmaceutically acceptable salt thereof.

In some embodiments, the compound of Formula (I) has formula:

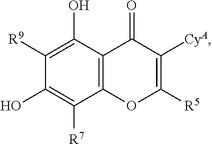

or a pharmaceutically acceptable salt thereof.

In some embodiments, the compound of Formula (I) has formula:

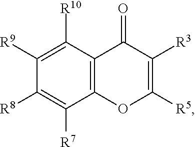

or a pharmaceutically acceptable salt thereof.

In some embodiments, the compound of Formula (I) has formula:

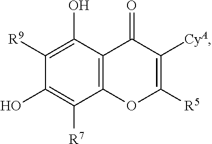

or a pharmaceutically acceptable salt thereof.

In some embodiments, the compound of Formula (I) has formula:

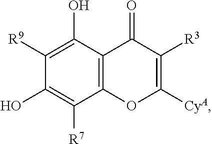

or a pharmaceutically acceptable salt thereof.

In some embodiments, the compound of Formula (I) has formula:

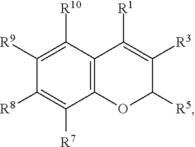

or a pharmaceutically acceptable salt thereof.

In some embodiments, the compound of Formula (I) has formula:

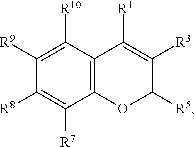

or a pharmaceutically acceptable salt thereof.

In some embodiments, the compound of Formula (I) has formula:

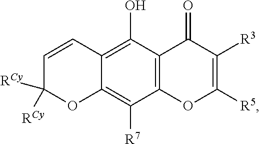

or a pharmaceutically acceptable salt thereof.

In some embodiments, the compound of Formula (I) has formula:

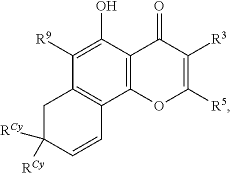

or a pharmaceutically acceptable salt thereof.

In some embodiments, the compound of Formula (I) has formula:

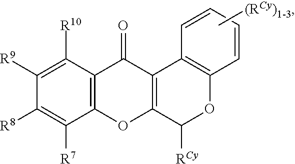

or a pharmaceutically acceptable salt thereof.

In some embodiments, the compound of Formula (I) has formula:

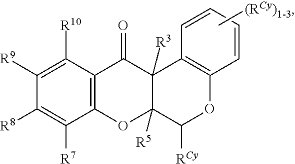

or a pharmaceutically acceptable salt thereof.

In some embodiments, the compound of Formula (I) has formula:

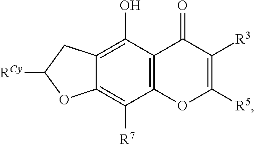

or a pharmaceutically acceptable salt thereof.

In some embodiments, the compound of Formula (I) has formula:

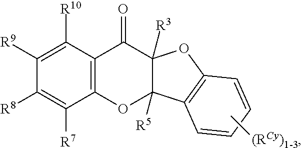

or a pharmaceutically acceptable salt thereof.

In some embodiments, the compound of Formula (I) has formula:

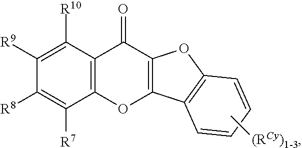

or a pharmaceutically acceptable salt thereof.

In some embodiments, the compound of Formula (I) has formula:

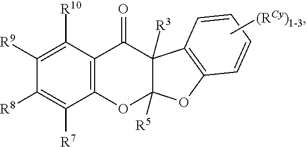

or a pharmaceutically acceptable salt thereof.

In some embodiments, the compound of Formula (I) has formula:

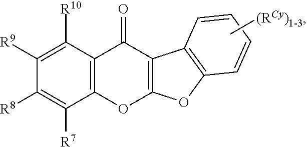

or a pharmaceutically acceptable salt thereof.

In some embodiments, the compound of Formula (I) comprises at least one (e.g., 1, 2, 3, 4, or 5) C₄₋₁₅ alkenyl group. In some embodiments, the compound of Formula (I) comprises at least one (e.g., 1, 2, 3, 4, or 5) prenyl group.

In some embodiments, a prenyl is a moiety having formula:

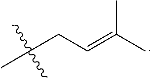 [SMILES: *CC=C(C)C]

In some embodiments, a prenyl is a moiety having formula:

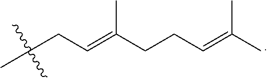 [SMILES: *C/C=C(\C)CCC=C(C)C]

In some embodiments, a prenyl is a moiety having formula:

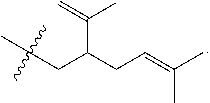 [SMILES: *CC(CC=C(C)C)C(=C)C]

In some embodiments, a prenyl group is optionally substituted with 1, 2 or 3 substituents independently selected from OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, NR^{c1}S(O)₂NR^{c1}R^{d1}, S(O)₂R^{b1}, and S(O)₂NR^{c1}R^{d1}.

In some embodiments, a prenyl group is optionally substituted with 1, 2 or 3 substituents independently selected from C₁₋₆ haloalkyl, halo, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}.

In some embodiments, the compound of Formula (I) is any one of the compounds listed in Table 1a, or a pharmaceutically acceptable salt thereof.

In some embodiments, the compound of Formula (I) is any one of the compounds listed in Table 1b, or a pharmaceutically acceptable salt thereof.

In some embodiments, the compound of Formula (I) is any one of the compounds listed in Table 1c, or a pharmaceutically acceptable salt thereof.

In some embodiments, the compound of Formula (I) is any one of the compounds listed in Table 1d, or a pharmaceutically acceptable salt thereof.

In some embodiments, the compound of Formula (I) is any one of the compounds listed in Table 1e, or a pharmaceutically acceptable salt thereof.

In some embodiments, the compound of Formula (I) is any one of the compounds listed in Table 1f, or a pharmaceutically acceptable salt thereof.

In some embodiments, the compound of Formula (I) is any one of the compounds listed in Table 1g, or a pharmaceutically acceptable salt thereof.

In some embodiments, the compound of Formula (I) is any one of the compounds listed in Table 2a, or a pharmaceutically acceptable salt thereof.

In some embodiments, the compound of Formula (I) is any one of the compounds listed in Table 2b, or a pharmaceutically acceptable salt thereof.

In some embodiments, the compound of Formula (I) is any one of the compounds listed in Table 2c, or a pharmaceutically acceptable salt thereof.

In some embodiments, the compound of Formula (I) is any one of the compounds listed in Table 2d, or a pharmaceutically acceptable salt thereof.

In some embodiments, the compound of Formula (I) is any one of the compounds listed in Table 2e, or a pharmaceutically acceptable salt thereof.

In some embodiments, the compound of Formula (I) is any one of the compounds listed in Table 3, or a pharmaceutically acceptable salt thereof.

In some embodiments, the compound of Formula (I) is selected from biflavone, ginkgetin, quercetin, luteolin, baicalin, amentoflavone, hesperetin, hesperidin, hervacetin, rhoifolin, pectolinarin, gallocatechin, gallocatechin gallate, diosmin, kaempferol, luteolin-7-glucoside, quercetin-3-β-galactoside, naringenin, apigenin-7-glucoside, catechin, pigallocatechin, isoquercetin, epigallocatechin, herbacetin, isotheaflavin, baicalein, theacitrin A, corilagin, theaflavin, (-)-taxifolin, Rhamnetin, quercetin 3-glucuronide-7-glucoside, quercetin 3-vicianoside, delphinidin 3-O-glucoside, petunidin 3-O-glucoside, chrysoeriol 8-C-glucoside, schaftoside, rutin, hypericin, cyanidin 3-glucoside, glabridin, orientin, astilbin, puerarin, astragalin, 2′-hydroxygenistein, genistein, morin, dihydromorin, steppogenin, 6-methoxyluteolin, pinocembrin 7-O-(3″-galloyl-4″,6″-(S)-hexahydroxydiphenoyl)-β-D-glucose (PGHG), biorobin, jaceidin triacetate, kaempferol-4′-O-beta-D-glucopyranoside, dracoflavan B1, isoschaftoside, norartocarpetin, 3′,4′,5,7-tetra hydroxyisoflavanone, and artocarpanone, or a pharmaceutically acceptable salt thereof.

In some embodiments, the compound of Formula (I) is selected from pinocembrin 7-O-(3″-galloyl-4″,6″-(S)-hexahydroxydiphenoyl)-β-D-glucose (PGHG), Amentoflavone, Jaceidin triacetate, 3,8′-Biapigenin, Baicalein, myricetin, apigenin, bilobetin, Ginkgetin, Isoginkgetin, Podocarpus flavone A, 2,3-dihydro-heveaflavone, Tetrahydro-amentoflavone, Apigenin 6-C-alpha-L-arabinopyranosyl-8-C-beta-D-xylopyranoside, 3,6-Dimethoxyapigenin, Apigenin 7-glucuronide, 7,4′-Di-O-methylapigenin 5-O-xylosylglucoside, Apigenin 4′-O-rhamnoside, Apigenin 5-O-neohesperidoside, 7,4′-Di-O-methylapigenin, and Pinocembrin.

In some embodiments, the present disclosure provides a pharmaceutical composition comprising a compound of Formula (I), or a pharmaceutically acceptable salt thereof.

Pharmaceutically Acceptable Salts

In some embodiments, a salt of a compound of Formula (I) is formed between an acid and a basic group of the compound, such as an amino functional group, or a base and an acidic group of the compound, such as a carboxyl functional group. According to another embodiment, the compound is a pharmaceutically acceptable acid addition salt.

In some embodiments, acids commonly employed to form pharmaceutically acceptable salts of the compounds of the present disclosure include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

In some embodiments, bases commonly employed to form pharmaceutically acceptable salts of the compounds of the present disclosure include hydroxides of alkali metals, including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C₁-C₆)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like. In some embodiments, the compounds of Formula (I), or pharmaceutically acceptable salts thereof, are substantially isolated.

Methods of Use

In some embodiments, the present disclosure provides a method of modulating activity of a main protease of a SAR-CoV-2 coronavirus in a cell, the method comprising contacting the cell with an effective amount of a compound of Formula (I), or a pharmaceutically acceptable salt thereof.

In some embodiments, the present disclosure provides a method of modulating activity of a main protease of a SAR-CoV coronavirus in a cell, the method comprising contacting the cell with an effective amount of a compound of Formula (I), or a pharmaceutically acceptable salt thereof.

In some embodiments, the present disclosure provides a method of modulating activity of a main protease of a MERS-CoV coronavirus in a cell, the method comprising contacting the cell with an effective amount of a compound of Formula (I), or a pharmaceutically acceptable salt thereof.

In some embodiments, the contacting is carried out in vitro. In some embodiments, the contacting is carried out in vivo. In some embodiments, the contacting is carried out ex vivo.

In some embodiments, the present disclosure provides a method of treating or preventing a viral infection caused by a SAR-CoV-2 coronavirus in a subject, the method comprising administering to the subject a therapeutically effective amount of a compound of Formula (I), or a pharmaceutically acceptable salt thereof. In some embodiments, the viral infection is coronavirus disease 2019 (COVID-19).

In some embodiments, the present disclosure provides a method of treating or preventing a viral infection caused by a SAR-CoV coronavirus in a subject, the method comprising administering to the subject a therapeutically effective amount of a compound of Formula (I), or a pharmaceutically acceptable salt thereof. In some embodiments, the viral infection is severe acute respiratory syndrome (SARS).

In some embodiments, the present disclosure provides a method of treating or preventing a viral infection caused by a MERS-CoV coronavirus in a subject, the method comprising administering to the subject a therapeutically effective amount of a compound of Formula (I), or a pharmaceutically acceptable salt thereof. In some embodiments, the viral infection is Middle East respiratory syndrome (MERS).

In some embodiments, the present disclosure provides a method of inhibiting protein disulfide isomerase (PDI) in a cell, the method comprising contacting the cell with an effective amount of a compound of Formula (I), or a pharmaceutically acceptable salt thereof. In some embodiments, the contacting is carried out in vitro. In some embodiments, the contacting is carried out in vivo. In some embodiments, the contacting is carried out ex vivo.

In some embodiments, the present disclosure provides a method of inhibiting protein disulfide isomerase (PDI) in a subject, the method comprising administering to the subject a therapeutically effective amount of a compound of Formula (I), or a pharmaceutically acceptable salt thereof, or a pharmaceutical composition comprising same. Hence, the present disclosure provides a method of inhibiting thrombosis in a subject, the method comprising administering to the subject a therapeutically effective amount of a compound of Formula (I), or a pharmaceutically acceptable salt thereof, or a pharmaceutical composition comprising same.

Protein disulfide isomerase (PDI) is essential for normal thrombosis. In some embodiments, the flavonoid PDI inhibitors of the present disclosure block thrombus formation in vivo and have shown efficacy as antithrombotics. Given the substantial morbidity and mortality caused by viral infection associated (e.g., COVID19-associated) coagulopathy, the flavonoids of this disclosure inhibits both SARS-CoV-2 Mpro and PDI, thus blocking both viral replication and thrombus formation.

In some embodiments, the compound of Formula (I), or a pharmaceutically acceptable salt thereof, can be administered to the subject by any suitable route disclosed herein, including orally or intranasally. In some embodiments, the compound of Formula (I) is administered to the subject intranasally, for example, using nasal sprays as described herein. In the nasal spray, a solution of the compound of Formula (I), or a pharmaceutically acceptable salt thereof, may be atomized into a fine aerosol mist. Alternatively, the compound of Formula (I), or a pharmaceutically acceptable salt thereof, can be spayed intranasally in a form of a dry powder having mean particle size of the compound of Formula (I), or a pharmaceutically acceptable salt thereof, from about 10 nm to about 100 μm.

Combination Therapies

In some embodiments, the compound of Formula (I), or a pharmaceutically acceptable salt thereof, can be administered to the subject in combination with at least one additional anti-viral agent, or a pharmaceutically acceptable salt thereof. Suitable examples of the additional anti-viral agents include favipiravir, remdesivir, lopinavir, ritonavir, remdesivir, rupintrivir, ribavirin, amantadine, rimantadine, pleconaril, molnupiravir, ivermectin, oleandrin, hydroxychloroquine, recombinant ACE2, and chloroquine, or a pharmaceutically acceptable salt thereof. Other suitable examples of additional anti-viral therapies include SAR-CoV antibodies, SARS-CoV-2 antibodies, and MERS-CoV antibodies. Examples of SARS-CoV-2 antibodies include REGN-COV2, LY-CoV555, and LY-CoV016. A compound of Formula (I), or a pharmaceutically acceptable salt thereof, can also be administered to the subject in combination with an immunomodulating drug, or a pharmaceutically acceptable salt thereof. Suitable examples of immunomodulating drugs include dexamethasone, SNG001, tocilizumab, baricitinib, convalescent plasma, interferons, glatiramer acetate, fingolimod, teriflunomide, and dimethyl fumarate, or a pharmaceutically acceptable salt thereof.

The compound of Formula (I), or a pharmaceutically acceptable salt thereof, may be administered to the patient simultaneously with the additional therapeutic agent (in the same pharmaceutical composition or dosage form or in different compositions or dosage forms) or consecutively (the additional therapeutic agent may be administered in a separate pharmaceutical composition or dosage form before or after administration of the compound of the present disclosure).

Kits

The present invention also includes pharmaceutical kits useful, for example, in the treatment of disorders, diseases and conditions referred to herein, which include one or more containers containing a pharmaceutical composition comprising a therapeutically effective amount of a compound of Formula (I), or a pharmaceutically acceptable salt thereof. Such kits can further include, if desired, one or more of various conventional pharmaceutical kit components, such as, for example, containers with one or more pharmaceutically acceptable carriers, additional containers, etc. Instructions, either as inserts or as labels, indicating quantities of the components to be administered, guidelines for administration, and/or guidelines for mixing the components, can also be included in the kit. The kit may optionally include an additional anti-viral agent as described herein.

Pharmaceutical Compositions and Formulations

The present application also provides pharmaceutical compositions comprising an effective amount of a compound of Formula (I), or a pharmaceutically acceptable salt thereof; and a pharmaceutically acceptable carrier. The pharmaceutical composition may also comprise any one of the additional therapeutic agents described herein. In certain embodiments, the application also provides pharmaceutical compositions and dosage forms comprising any one the additional therapeutic agents described herein. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of the present application include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

The compositions or dosage forms may contain any one of the compounds and therapeutic agents described herein in the range of 0.005% to 100% with the balance made up from the suitable pharmaceutically acceptable excipients. The contemplated compositions may contain 0.001%-100% of any one of the compounds and therapeutic agents provided herein, in one embodiment 0.1-⁹⁵%, in another embodiment 75-85%, in a further embodiment 20-80%, wherein the balance may be made up of any pharmaceutically acceptable excipient described herein, or any combination of these excipients.

Routes of Administration and Dosage Forms

The pharmaceutical compositions of the present application include those suitable for any acceptable route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intranasal, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal.

Compositions and formulations described herein may conveniently be presented in a unit dosage form, e.g., tablets, sustained release capsules, and in liposomes, and may be prepared by any methods well known in the art of pharmacy. See, for example, Remington: The Science and Practice of Pharmacy, Lippincott Williams & Wilkins, Baltimore, MD (20th ed. 2000). Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In some embodiments, any one of the compounds and therapeutic agents disclosed herein are administered orally. Compositions of the present application suitable for oral administration may be presented as discrete units such as capsules, sachets, granules or tablets each containing a predetermined amount (e.g., effective amount) of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption. In the case of tablets for oral use, carriers that are commonly used include lactose, sucrose, glucose, mannitol, and silicic acid and starches. Other acceptable excipients may include: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quatemary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. For oral administration in a capsule form, useful diluents include lactose and dried corn starch. When aqueous suspensions are administered orally, the active ingredient is combined with emulsifying and suspending agents. If desired, certain sweetening and/or flavoring and/or coloring agents may be added. Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions or infusion solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, saline (e.g., 0.9% saline solution) or 5% dextrose solution, immediately prior to use. Extemporaneous injection solutions and suspensions may be prepared from sterile powders, granules and tablets. The injection solutions may be in the form, for example, of a sterile injectable aqueous or oleaginous suspension. This suspension may be formulated according to techniques known in the art using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of the present application may be administered in the form of suppositories for rectal administration. These compositions can be prepared by mixing a compound of the present application with a suitable non-irritating excipient which is solid at room temperature but liquid at the rectal temperature and therefore will melt in the rectum to release the active components. Such materials include, but are not limited to, cocoa butter, beeswax, and polyethylene glycols.

The pharmaceutical compositions of the present application may be administered intranasally, e.g., by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, for example, U.S. Pat. No. 6,803,031. Additional formulations and methods for intranasal administration are found in Ilium, L., J Pharm Pharmacol, 56:3-17, 2004 and Ilium, L., Eur J Pharm Sci 11:1-18, 2000.

The topical compositions of the present disclosure can be prepared and used in the form of an aerosol spray, cream, emulsion, solid, liquid, dispersion, foam, oil, gel, hydrogel, lotion, mousse, ointment, powder, patch, pomade, solution, pump spray, stick, towelette, soap, or other forms commonly employed in the art of topical administration and/or cosmetic and skin care formulation. The topical compositions can be in an emulsion form. Topical administration of the pharmaceutical compositions of the present application is especially useful when the desired treatment involves areas or organs readily accessible by topical application. In some embodiments, the topical composition comprises a combination of any one of the compounds and therapeutic agents disclosed herein, and one or more additional ingredients, carriers, excipients, or diluents including, but not limited to, absorbents, anti-irritants, anti-acne agents, preservatives, antioxidants, coloring agents/pigments, emollients (moisturizers), emulsifiers, film-forming/holding agents, fragrances, leave-on exfoliants, prescription drugs, preservatives, scrub agents, silicones, skin-identical/repairing agents, slip agents, sunscreen actives, surfactants/detergent cleansing agents, penetration enhancers, and thickeners.

Dosages and Regimens

In the pharmaceutical compositions of the present application, a compound of Formula (I), or a pharmaceutically acceptable salt thereof, is present in an effective amount (e.g., a therapeutically effective amount). Effective doses may vary, depending on the diseases treated, the severity of the disease, the route of administration, the sex, age and general health condition of the subject, excipient usage, the possibility of co-usage with other therapeutic treatments such as use of other agents and the judgment of the treating physician.

In some embodiments, an effective amount of the compound of Formula (I), or a pharmaceutically acceptable salt thereof, can range, for example, from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0.1 mg/kg to about 200 mg/kg; from about 0.1 mg/kg to about 150 mg/kg; from about 0.1 mg/kg to about 100 mg/kg; from about 0.1 mg/kg to about 50 mg/kg; from about 0.1 mg/kg to about 10 mg/kg; from about 0.1 mg/kg to about 5 mg/kg; from about 0.1 mg/kg to about 2 mg/kg; from about 0.1 mg/kg to about 1 mg/kg; or from about 0.1 mg/kg to about 0.5 mg/kg). In some embodiments, an effective amount of a compound of Formula (I) is about 0.1 mg/kg, about 0.5 mg/kg, about 1 mg/kg, about 2 mg/kg, or about 5 mg/kg.

The foregoing dosages can be administered on a daily basis (e.g., as a single dose or as two or more divided doses, e.g., once daily, twice daily, thrice daily) or non-daily basis (e.g., every other day, every two days, every three days, once weekly, twice weekly, once every two weeks, once a month).

### Definitions

As used herein, the term “about” means “approximately” (e.g., plus or minus approximately 10% of the indicated value).

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

At various places in the present specification various aryl, heteroaryl, cycloalkyl, and heterocycloalkyl rings are described. Unless otherwise specified, these rings can be attached to the rest of the molecule at any ring member as permitted by valency. For example, the term “a pyridine ring” or “pyridinyl” may refer to a pyridin-2-yl, pyridin-3-yl, or pyridin-4-yl ring.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

The term “aromatic” refers to a carbocycle or heterocycle having one or more polyunsaturated rings having aromatic character (i.e., having (4n+2) delocalized π (pi) electrons where n is an integer).

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

Throughout the definitions, the term “Cₙ₋ₘ” indicates a range which includes the endpoints, wherein n and m are integers and indicate the number of carbons. Examples include C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms.

As used herein, “Cₙ₋ₘ alkenyl” refers to an alkyl group having one or more double carbon-carbon bonds and having n to m carbons. Example alkenyl groups include, but are not limited to, ethenyl, n-propenyl, isopropenyl, n-butenyl, sec-butenyl, prenyl group, geranyl group, and the like. In some embodiments, the alkenyl moiety contains 2 to 6, 2 to 4, 2 to 3, 4 to 15, or 5 to 15 carbon atoms.

As used herein, “Cₙ₋ₘ alkynyl” refers to an alkyl group having one or more triple carbon-carbon bonds and having n to m carbons. Example alkynyl groups include, but are not limited to, ethynyl, propyn-1-yl, propyn-2-yl, and the like. In some embodiments, the alkynyl moiety contains 2 to 6, 2 to 4, or 2 to 3 carbon atoms.

As used herein, the term “Cₙ₋ₘ alkylene”, employed alone or in combination with other terms, refers to a divalent alkyl linking group having n to m carbons. Examples of alkylene groups include, but are not limited to, ethan-1,1-diyl, ethan-1,2-diyl, propan-1,1-diyl, propan-1,3-diyl, propan-1,2-diyl, butan-1,4-diyl, butan-1,3-diyl, butan-1,2-diyl, 2-methyl-propan-1,3-diyl, and the like. In some embodiments, the alkylene moiety contains 2 to 6, 2 to 4, 2 to 3, 1 to 6, 1 to 4, or 1 to 2 carbon atoms.

As used herein, the term “Cₙ₋ₘ alkoxy”, employed alone or in combination with other terms, refers to a group of formula —O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, “Cₙ₋ₘ haloalkoxy” refers to a group of formula —O-haloalkyl having n to m carbon atoms. An example haloalkoxy group is OCF₃. In some embodiments, the haloalkoxy group is fluorinated only. In some embodiments, the alkyl group has 1 to 6, 1 to 4, or 1 to 3 carbon atoms.

As used herein, the term “amino” refers to a group of formula —NH₂.

As used herein, the term “Cₙ₋ₘ alkylamino” refers to a group of formula —NH(alkyl), wherein the alkyl group has n to m carbon atoms. In some embodiments, the alkyl group has 1 to 6, 1 to 4, or 1 to 3 carbon atoms. Examples of alkylamino groups include, but are not limited to, N-methylamino, N-ethylamino, N-propylamino (e.g., N-(n-propyl)amino and N-isopropylamino), N-butylamino (e.g., N-(n-butyl)amino and N-(tert-butyl)amino), and the like.

As used herein, the term “di(Cₙ₋ₘ-alkyl)amino” refers to a group of formula —N(alkyl)₂, wherein the two alkyl groups each has, independently, n to m carbon atoms. In some embodiments, each alkyl group independently has 1 to 6, 1 to 4, or 1 to 3 carbon atoms.

As used herein, the term “Cₙ₋ₘ alkoxycarbonyl” refers to a group of formula —C(O)O-alkyl, wherein the alkyl group has n to m carbon atoms. In some embodiments, the alkyl group has 1 to 6, 1 to 4, or 1 to 3 carbon atoms. Examples of alkoxycarbonyl groups include, but are not limited to, methoxycarbonyl, ethoxycarbonyl, propoxycarbonyl (e.g., n-propoxycarbonyl and isopropoxycarbonyl), butoxycarbonyl (e.g., n-butoxycarbonyl and tert-butoxycarbonyl), and the like.

As used herein, the term “Cₙ₋ₘ alkylcarbonyl” refers to a group of formula —C(O)-alkyl, wherein the alkyl group has n to m carbon atoms. In some embodiments, the alkyl group has 1 to 6, 1 to 4, or 1 to 3 carbon atoms. Examples of alkylcarbonyl groups include, but are not limited to, methylcarbonyl, ethylcarbonyl, propylcarbonyl (e.g., n-propylcarbonyl and isopropylcarbonyl), butylcarbonyl (e.g., n-butylcarbonyl and tert-butylcarbonyl), and the like.

As used herein, the term “Cₙ₋ₘ alkylcarbonylamino” refers to a group of formula —NHC(O)-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfonylamino” refers to a group of formula —NHS(O)₂-alkyl, wherein the alkyl group has n to m carbon atoms. In some embodiments, the alkyl group has 1 to 6, 1 to 4, or 1 to 3 carbon atoms.

As used herein, the term “aminosulfonyl” refers to a group of formula —S(O)₂NH₂.

As used herein, the term “Cₙ₋ₘ alkylaminosulfonyl” refers to a group of formula —S(O)₂NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminosulfonyl” refers to a group of formula —S(O)₂N(alkyl)₂, wherein each alkyl group independently has n to m carbon atoms. In some embodiments, each alkyl group has, independently, 1 to 6, 1 to 4, or 1 to 3 carbon atoms.

As used herein, the term “aminosulfonylamino” refers to a group of formula —NHS(O)₂NH₂.

As used herein, the term “Cₙ₋ₘ alkylaminosulfonylamino” refers to a group of formula —NHS(O)₂NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminosulfonylamino” refers to a group of formula —NHS(O)₂N(alkyl)₂, wherein each alkyl group independently has n to m carbon atoms. In some embodiments, each alkyl group has, independently, 1 to 6, 1 to 4, or 1 to 3 carbon atoms.

As used herein, the term “aminocarbonylamino”, employed alone or in combination with other terms, refers to a group of formula —NHC(O)NH₂.

As used herein, the term “Cₙ₋ₘ alkylaminocarbonylamino” refers to a group of formula —NHC(O)NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminocarbonylamino” refers to a group of formula —NHC(O)N(alkyl)₂, wherein each alkyl group independently has n to m carbon atoms. In some embodiments, each alkyl group has, independently, 1 to 6, 1 to 4, or 1 to 3 carbon atoms.

As used herein, the term “carbamyl” to a group of formula —C(O)NH₂.

As used herein, the term “Cₙ₋ₘ alkylcarbamyl” refers to a group of formula —C(O)—NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ-alkyl)carbamyl” refers to a group of formula —C(O)N(alkyl)₂, wherein the two alkyl groups each has, independently, n to m carbon atoms. In some embodiments, each alkyl group independently has 1 to 6, 1 to 4, or 1 to 3 carbon atoms.

As used herein, the term “thio” refers to a group of formula —SH.

As used herein, the term “Cₙ₋ₘ alkylthio” refers to a group of formula —S-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfinyl” refers to a group of formula —S(O)-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfonyl” refers to a group of formula —S(O)₂-alkyl, wherein the alkyl group has n to m carbon atoms. In some embodiments, the alkyl group has 1 to 6, 1 to 4, or 1 to 3 carbon atoms.

As used herein, the term “carbonyl”, employed alone or in combination with other terms, refers to a —C(═O)— group, which may also be written as C(O).

As used herein, the term “carboxy” refers to a —C(O)OH group.

As used herein, the term “cyano-C₁₋₃ alkyl” refers to a group of formula —(C₁₋₃ alkylene)-CN.

As used herein, the term “HO—C₁₋₃ alkyl” refers to a group of formula —(C₁₋₃ alkylene)-OH.

As used herein, “halo” refers to F, Cl, Br, or I. In some embodiments, a halo is F, Cl, or Br.

As used herein, the term “aryl,” employed alone or in combination with other terms, refers to an aromatic hydrocarbon group, which may be monocyclic or polycyclic (e.g., having 2, 3 or 4 fused rings). The term “Cₙ₋ₘ aryl” refers to an aryl group having from n to m ring carbon atoms. Aryl groups include, e.g., phenyl, naphthyl, anthracenyl, phenanthrenyl, indanyl, indenyl, and the like. In some embodiments, aryl groups have from 6 to 10 carbon atoms. In some embodiments, the aryl group is phenyl or naphtyl.

As used herein, “cycloalkyl” refers to non-aromatic cyclic hydrocarbons including cyclized alkyl and/or alkenyl groups. Cycloalkyl groups can include mono- or polycyclic (e.g., having 2, 3 or 4 fused rings) groups and spirocycles. Ring-forming carbon atoms of a cycloalkyl group can be optionally substituted by 1 or 2 independently selected oxo or sulfide groups (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, or 10 ring-forming carbons (C₃₋₁₀). In some embodiments, the cycloalkyl is a C₃₋₁₀ monocyclic or bicyclic cyclocalkyl. In some embodiments, the cycloalkyl is a C₃₋₇ monocyclic cyclocalkyl. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbomyl, norpinyl, norcamyl, adamantyl, and the like. In some embodiments, cycloalkyl is cyclopropyl, cyclobutyl, cyclopentyl, or cyclohexyl.

As used herein, “heteroaryl” refers to a monocyclic or polycyclic aromatic heterocycle having at least one heteroatom ring member selected from sulfur, oxygen, and nitrogen. In some embodiments, the heteroaryl ring has 1, 2, 3, or 4 heteroatom ring members independently selected from nitrogen, sulfur and oxygen. In some embodiments, any ring-forming N in a heteroaryl moiety can be an N-oxide. In some embodiments, the heteroaryl is a 5-10 membered monocyclic or bicyclic heteroaryl having 1, 2, 3 or 4 heteroatom ring members independently selected from nitrogen, sulfur and oxygen. In some embodiments, the heteroaryl is a 5-6 monocyclic heteroaryl having 1 or 2 heteroatom ring members independently selected from nitrogen, sulfur and oxygen. In some embodiments, the heteroaryl is a five-membered or six-membered heteroaryl ring. A five-membered heteroaryl ring is a heteroaryl with a ring having five ring atoms wherein one or more (e.g., 1, 2, or 3) ring atoms are independently selected from N, O, and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. A six-membered heteroaryl ring is a heteroaryl with a ring having six ring atoms wherein one or more (e.g., 1, 2, or 3) ring atoms are independently selected from N, O, and S. Exemplary six-membered ring heteroaryls are pyridyl, pyrazinyl, pyrimidinyl, triazinyl and pyridazinyl.

As used herein, “heterocycloalkyl” refers to non-aromatic monocyclic or polycyclic heterocycles having one or more ring-forming heteroatoms selected from O, N, or S. Included in heterocycloalkyl are monocyclic 4-, 5-, 6-, 7-, 8-, 9- or 10-membered heterocycloalkyl groups. Heterocycloalkyl groups can also include spirocycles. Example heterocycloalkyl groups include pyrrolidin-2-one, 1,3-isoxazolidin-2-one, pyranyl, tetrahydropuran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, and the like. Ring-forming carbon atoms and heteroatoms of a heterocycloalkyl group can be optionally substituted by 1 or 2 independently selected oxo or sulfido groups (e.g., C(O), S(O), C(S), or S(O)₂, etc.). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the cycloalkyl ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl is a monocyclic 4-6 membered heterocycloalkyl having 1 or 2 heteroatoms independently selected from nitrogen, oxygen, or sulfur and having one or more oxidized ring members. In some embodiments, the heterocycloalkyl is a monocyclic or bicyclic 4-10 membered heterocycloalkyl having 1, 2, 3, or 4 heteroatoms independently selected from nitrogen, oxygen, or sulfur and having one or more oxidized ring members.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

As used herein, the term “oxo” refers to an oxygen atom as a divalent substituent, forming a carbonyl group when attached to a carbon (e.g., C═O), or attached to a heteroatom forming a sulfoxide or sulfone group.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, N═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. In some embodiments, the compound has the (R)-configuration. In some embodiments, the compound has the (S)-configuration.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone—enol pairs, amide—imidic acid pairs, lactam—lactim pairs, enamine—imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” a viral main protease with a compound of the invention includes the administration of a compound of the present invention to an individual or patient, such as a human, having a viral main protease, as well as, for example, introducing a compound of the invention into a sample containing a cellular or purified preparation containing the viral main protease.

As used herein, the term “individual”, “patient”, or “subject” used interchangeably, refers to any animal, including mammals, preferably mice, rats, other rodents, rabbits, dogs, cats, swine, cattle, sheep, horses, or primates, and most preferably humans.

As used herein, the phrase “effective amount” or “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein the term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatology), or 2) ameliorating the disease; for example, ameliorating a disease, condition or disorder in an individual who is experiencing or displaying the pathology or symptomatology of the disease, condition or disorder (i.e., reversing the pathology and/or symptomatology).

As used herein, the term “preventing” or “prevention” of a disease, condition or disorder refers to decreasing the risk of occurrence of the disease, condition or disorder in a subject or group of subjects (e.g., a subject or group of subjects predisposed to or susceptible to the disease, condition or disorder). In some embodiments, preventing a disease, condition or disorder refers to decreasing the possibility of acquiring the disease, condition or disorder and/or its associated symptoms. In some embodiments, preventing a disease, condition or disorder refers to completely or almost completely stopping the disease, condition or disorder from occurring.

### Examples

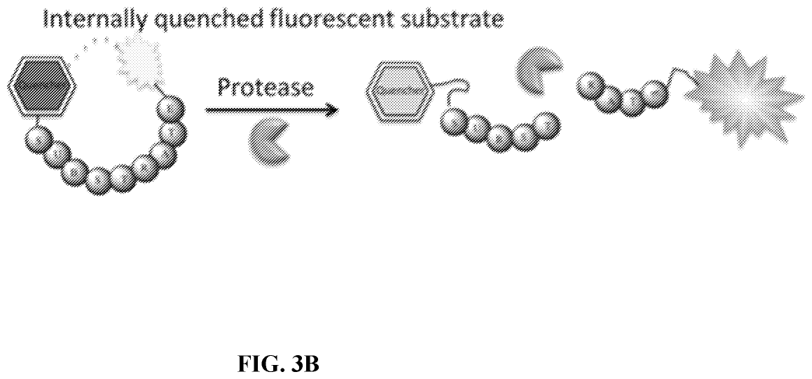

Assay details: A SARS-Cov-2 M^{pro} construct was prepared, which includes a His-tag and PreScission cleavage site (see FIG. 3A). The construct was transformed into E. coli and the enzyme was isolated using Ni²⁺-affinity chromatography. The C-terminal His tag was cleaved by PreScission and the N-term was autocleaved by the protease itself. The protein was further purified by FPLC and purity was assessed by gel electrophoresis and Coomassie staining. The enzymatic activity of purified SARS-CoV-2 M^{pro} was tested using a dabcyl-KTSAVLQ↓SGFRKME(Edans)-NH₂ substrate (GL Biochem) by monitoring at em 460 nm with ex 360 nm (the assay is schematically shown in FIG. 3B, adapted from Kasperkeiwicz et al., FEBS J, 284 (10), 1518-1539). Initial velocities were determined from the linear portion of the kinetic curve. As a quality control measure, the Kₘ and k_{cat} of purified SARS-CoV-2 M^{pro} were compared to published values and only preparations that show similar activity were used in the activity assay. SARS-CoV-2 M^{pro} was incubated with a test compound for 20 minutes prior to addition of substrate. Compounds were tested at 17 μM in duplicates. For each tested compound, assay results were reported as % fluorescence generated by initial enzyme activity (initial enzyme activity in the absence of test compound). Assay results are reported in Tables 1a-3. “+++” means <10% enzyme activity as indicated by fluorescence measurements, “++” means enzyme activity as indicated by fluorescence measurements ≥10% and <80%, and “+” means ≥80% enzyme activity as indicated by fluorescence measurements. IC₅₀ values were obtained by evaluating compounds at 0.015, 0.046, 0.14, 0.41, 1.23, 3.7, 11, 33, 100, 300 μM compound.

### Example 1—prenylated flavones, flavanones, and flavenes

TABLE 1a

| structure | Name | Activity | IC₅₀ |
|---|---|---|---|
| 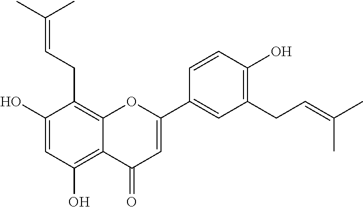 [SMILES: CC(C)=CCc1cc(-c2cc(=O)c3c(O)cc(O)c(CC=C(C)C)c3o2)ccc1O] | 8,3′-Diprenylapigenin | +++ | n/a |
| 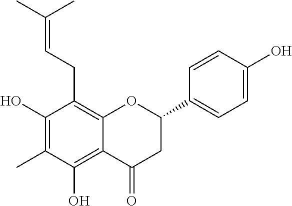 [SMILES: CC(C)=CCc1c(O)c(C)c(O)c2c1O[C@H](c1ccc(O)cc1)CC2=O] | 6-Methyl-8- prenylnaringenin | +++ | n/a |
| 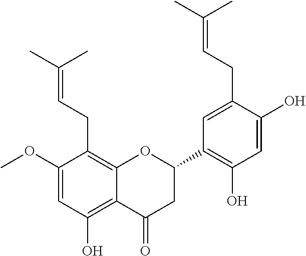 [SMILES: COc1cc(O)c2c(c1CC=C(C)C)O[C@H](c1cc(CC=C(C)C)c(O)cc1O)CC2=O] | Maackiaflavanone | +++ | 3 |
| 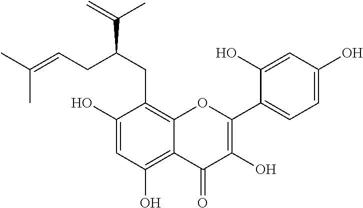 [SMILES: C=C(C)[C@H](CC=C(C)C)Cc1c(O)cc(O)c2c(=O)c(O)c(-c3ccc(O)cc3O)oc12] | Kushenol C | ++ | 5.5 |
| 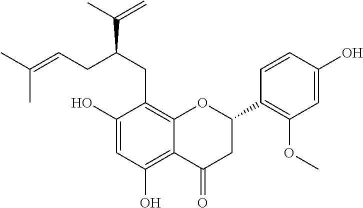 [SMILES: C=C(C)[C@H](CC=C(C)C)Cc1c(O)cc(O)c2c1O[C@H](c1ccc(O)cc1OC)CC2=O] | Isokurarinone | ++ | n/a |
| 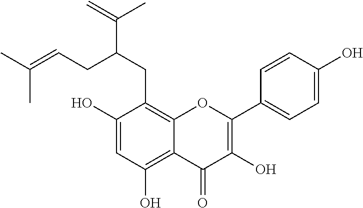 [SMILES: C=C(C)C(CC=C(C)C)Cc1c(O)cc(O)c2c(=O)c(O)c(-c3ccc(O)cc3)oc12] | 8- Lavandulylkaempferol | ++ | 19.5 |
| 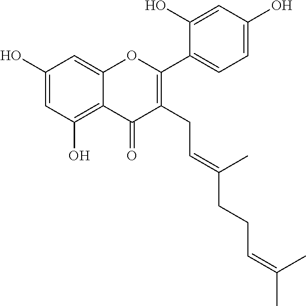 [SMILES: CC(C)=CCC/C(C)=C/Cc1c(-c2ccc(O)cc2O)oc2cc(O)cc(O)c2c1=O] | 5,7,2′,4′- Tetrahydroxy-3- geranylflavone | ++ | 5 |
| 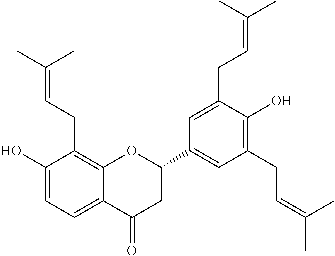 [SMILES: CC(C)=CCc1cc([C@@H]2CC(=O)c3ccc(O)c(CC=C(C)C)c3O2)cc(CC=C(C)C)c1O] | (−)-Sophoranone | ++ | n/a |
| 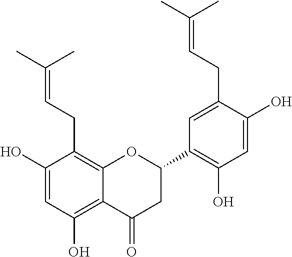 [SMILES: CC(C)=CCc1cc([C@@H]2CC(=O)c3c(O)cc(O)c(CC=C(C)C)c3O2)c(O)cc1O] | Euchrestaflavanone B | ++ | 96 |
| 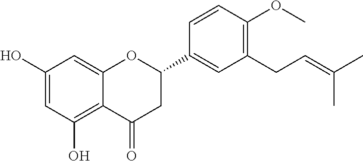 [SMILES: COc1ccc([C@@H]2CC(=O)c3c(O)cc(O)cc3O2)cc1CC=C(C)C] | 4′-O- Methyllicoflavanone | ++ | n/a |
| 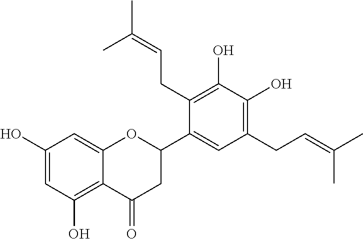 [SMILES: CC(C)=CCc1cc(C2CC(=O)c3c(O)cc(O)cc3O2)c(CC=C(C)C)c(O)c1O] | (+/−)-Sigmoidin A | ++ | n/a |
| 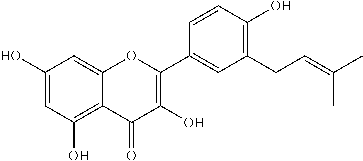 [SMILES: CC(C)=CCc1cc(-c2oc3cc(O)cc(O)c3c(=O)c2O)ccc1O] | Isolicoflavonol | ++ | n/a |
| 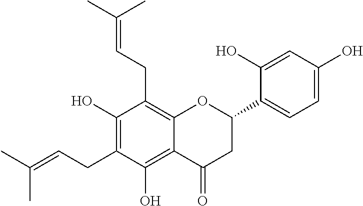 [SMILES: CC(C)=CCc1c(O)c(CC=C(C)C)c2c(c1O)C(=O)C[C@@H](c1ccc(O)cc1O)O2] | Kushenol E | ++ | n/a |
| 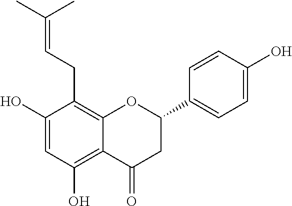 [SMILES: CC(C)=CCc1c(O)cc(O)c2c1O[C@H](c1ccc(O)cc1)CC2=O] | 8-Prenylnaringenin | ++ | n/a |
| 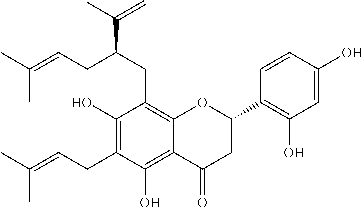 [SMILES: C=C(C)[C@H](CC=C(C)C)Cc1c(O)c(CC=C(C)C)c(O)c2c1O[C@H](c1ccc(O)cc1O)CC2=O] | Kushenol B | + | n/a |
| 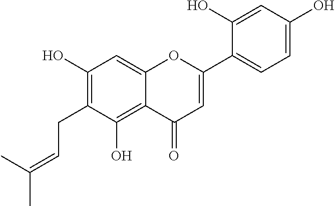 [SMILES: CC(C)=CCc1c(O)cc2oc(-c3ccc(O)cc3O)cc(=O)c2c1O] | Artocarpesin | + | n/a |
| 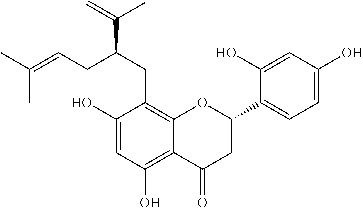 [SMILES: C=C(C)[C@H](CC=C(C)C)Cc1c(O)cc(O)c2c1O[C@H](c1ccc(O)cc1O)CC2=O] | Kushenol F | + | n/a |
| 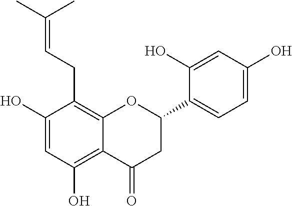 [SMILES: CC(C)=CCc1c(O)cc(O)c2c1O[C@H](c1ccc(O)cc1O)CC2=O] | Leachianone G | + | n/a |
| 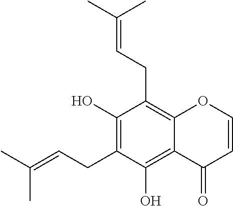 [SMILES: CC(C)=CCc1c(O)c(CC=C(C)C)c2occc(=O)c2c1O] | Eriosemation | + | n/a |
| 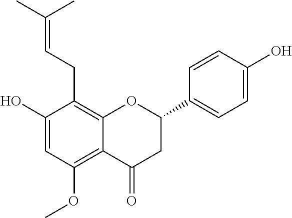 [SMILES: COc1cc(O)c(CC=C(C)C)c2c1C(=O)C[C@@H](c1ccc(O)cc1)O2] | Isoxanthohumol | + | n/a |
| 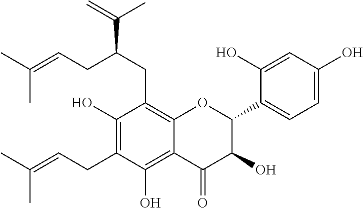 [SMILES: C=C(C)[C@H](CC=C(C)C)Cc1c(O)c(CC=C(C)C)c(O)c2c1O[C@H](c1ccc(O)cc1O)[C@@H](O)C2=O] | kushenol M | + | n/a |
| 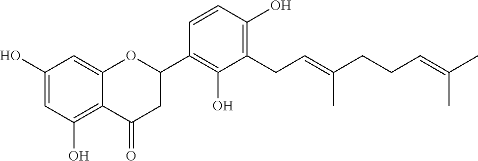 [SMILES: CC(C)=CCC/C(C)=C/Cc1c(O)ccc(C2CC(=O)c3c(O)cc(O)cc3O2)c1O] | sanggenol A | ++ | n/a |
| 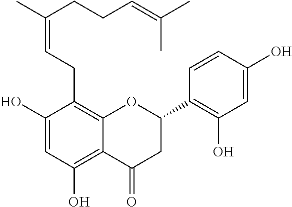 [SMILES: CC(C)=CCC/C(C)=C\Cc1c(O)cc(O)c2c1O[C@H](c1ccc(O)cc1O)CC2=O] | sophoraflavanone C | ++ | 63 |
| 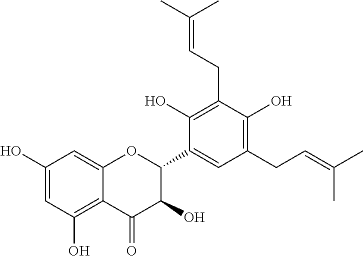 [SMILES: CC(C)=CCc1cc([C@H]2Oc3cc(O)cc(O)c3C(=O)[C@@H]2O)c(O)c(CC=C(C)C)c1O] | cathayanon I | ++ | n/a |
| 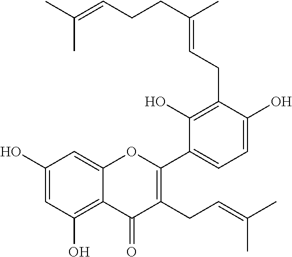 [SMILES: CC(C)=CCC/C(C)=C/Cc1c(O)ccc(-c2oc3cc(O)cc(O)c3c(=O)c2CC=C(C)C)c1O] | 3′-Geranyl-3-prenyl- 2′,4′,5,7- tetrahydroxyflavone | ++ | n/a |
| 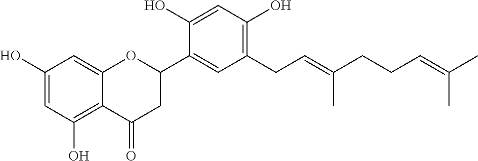 [SMILES: CC(C)=CCC/C(C)=C/Cc1cc(C2CC(=O)c3c(O)cc(O)cc3O2)c(O)cc1O] | kuwanon E | ++ | n/a |
| 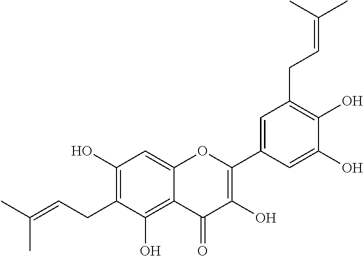 [SMILES: CC(C)=CCc1cc(-c2oc3cc(O)c(CC=C(C)C)c(O)c3c(=O)c2O)cc(O)c1O] | broussonol E | + | n/a |
| 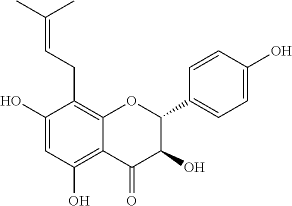 [SMILES: CC(C)=CCc1c(O)cc(O)c2c1O[C@H](c1ccc(O)cc1)[C@@H](O)C2=O] | neophellamuretin | + | n/a |
| 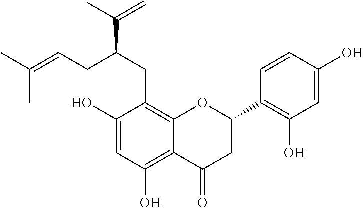 [SMILES: C=C(C)[C@H](CC=C(C)C)Cc1c(O)cc(O)c2c1O[C@H](c1ccc(O)cc1O)CC2=O] | Sophoraflavanone G | + | n/a |
| 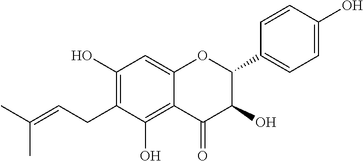 [SMILES: CC(C)=CCc1c(O)cc2c(c1O)C(=O)[C@H](O)[C@@H](c1ccc(O)cc1)O2] | Shuterin | + | n/a |
| 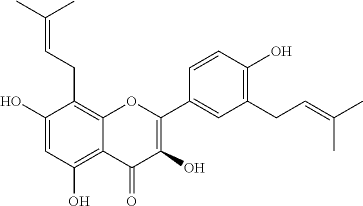 | Broussoflavonol F | + | n/a |
| 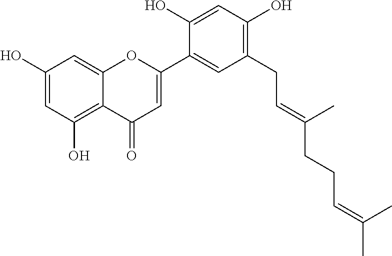 [SMILES: CC(C)=CCC/C(C)=C/Cc1cc(-c2cc(=O)c3c(O)cc(O)cc3o2)c(O)cc1O] | 5′-Geranyl-5,7,2′,4′- tetrahydroxyflavone | + | n/a |
| 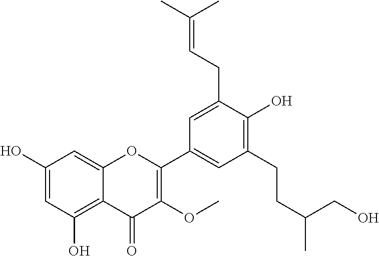 [SMILES: COc1c(-c2cc(CC=C(C)C)c(O)c(CCC(C)CO)c2)oc2cc(O)cc(O)c2c1=O] | Dodoviscin H | + | n/a |
| 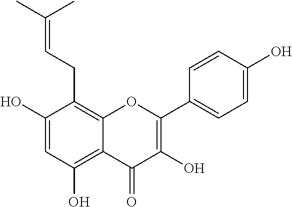 [SMILES: CC(C)=CCc1c(O)cc(O)c2c(=O)c(O)c(-c3ccc(O)cc3)oc12] | 8-Prenylkaempferol | + | n/a |
| 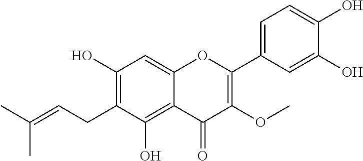 [SMILES: COc1c(-c2ccc(O)c(O)c2)oc2cc(O)c(CC=C(C)C)c(O)c2c1=O] | 6-Prenylquercetin-3- methylether | + | n/a |
| 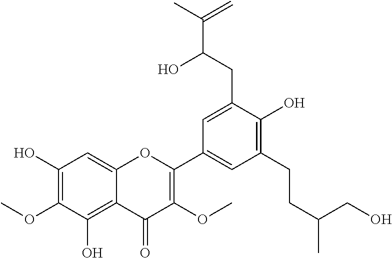 [SMILES: C=C(C)C(O)Cc1cc(-c2oc3cc(O)c(OC)c(O)c3c(=O)c2OC)cc(CCC(C)CO)c1O] | Dodoviscin A | + | n/a |
| 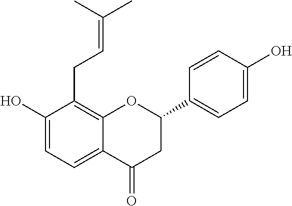 [SMILES: CC(C)=CCc1c(O)ccc2c1O[C@H](c1ccc(O)cc1)CC2=O] | Isobavachin | + | n/a |
| 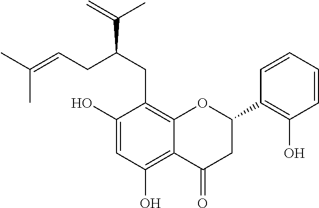 [SMILES: C=C(C)[C@H](CC=C(C)C)Cc1c(O)cc(O)c2c1O[C@H](c1ccccc1O)CC2=O] | Kushenol A | + | n/a |
| 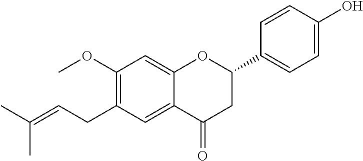 [SMILES: COc1cc2c(cc1CC=C(C)C)C(=O)C[C@@H](c1ccc(O)cc1)O2] | Bavachinin | + | n/a |
| 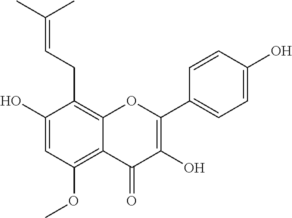 [SMILES: COc1cc(O)c(CC=C(C)C)c2oc(-c3ccc(O)cc3)c(O)c(=O)c12] | Sophoflavescenol | + | n/a |
| 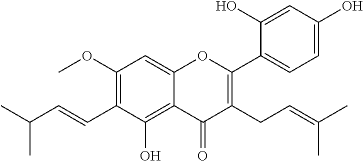 [SMILES: COc1cc2oc(-c3ccc(O)cc3O)c(CC=C(C)C)c(=O)c2c(O)c1/C=C/C(C)C] | Artocarpin | + | n/a |
| 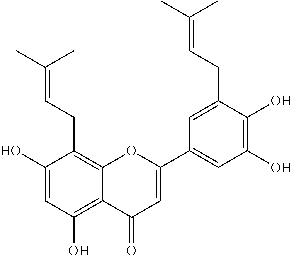 [SMILES: CC(C)=CCc1cc(-c2cc(=O)c3c(O)cc(O)c(CC=C(C)C)c3o2)cc(O)c1O] | Epimedokoreanin B | + | n/a |
| 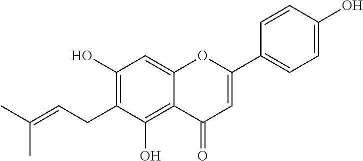 [SMILES: CC(C)=CCc1c(O)cc2oc(-c3ccc(O)cc3)cc(=O)c2c1O] | 4′,5,7-Trihydroxy-6- prenylflavone | + | n/a |
| 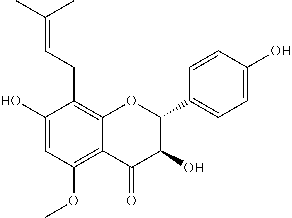 [SMILES: COc1cc(O)c(CC=C(C)C)c2c1C(=O)[C@H](O)[C@@H](c1ccc(O)cc1)O2] | 3,7,4′-Trihydroxy-5- methoxy-8- prenylflavanone | + | n/a |
| 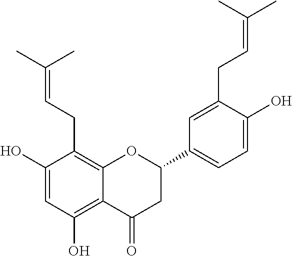 [SMILES: CC(C)=CCc1cc([C@@H]2CC(=O)c3c(O)cc(O)c(CC=C(C)C)c3O2)ccc1O] | Euchrestaflavanone A | + | n/a |
| 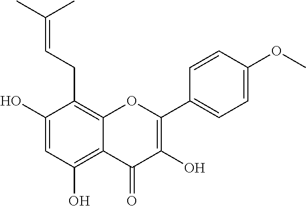 [SMILES: COc1ccc(-c2oc3c(CC=C(C)C)c(O)cc(O)c3c(=O)c2O)cc1] | Icaritin | + | n/a |
| 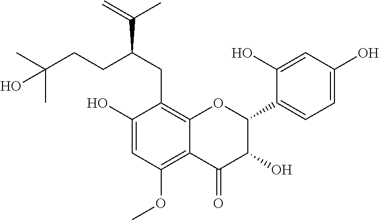 [SMILES: C=C(C)[C@H](CCC(C)(C)O)Cc1c(O)cc(OC)c2c1O[C@H](c1ccc(O)cc1O)[C@H](O)C2=O] | Kushenol K | + | n/a |
| 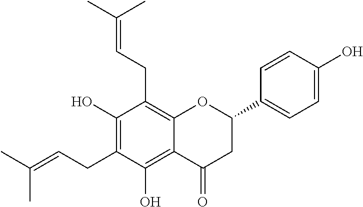 [SMILES: CC(C)=CCc1c(O)c(CC=C(C)C)c2c(c1O)C(=O)C[C@@H](c1ccc(O)cc1)O2] | 6,8- Diprenylnaringenin | + | n/a |
| 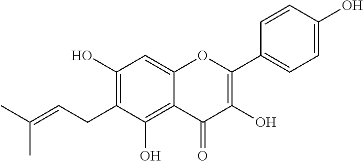 [SMILES: CC(C)=CCc1c(O)cc2oc(-c3ccc(O)cc3)c(O)c(=O)c2c1O] | Licoflavonol | + | n/a |
| 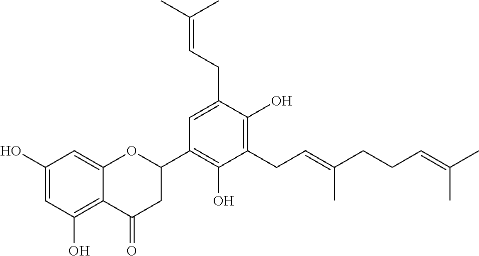 [SMILES: CC(C)=CCC/C(C)=C/Cc1c(O)c(CC=C(C)C)cc(C2CC(=O)c3c(O)cc(O)cc3O2)c1O] | Sanggenol P | + | n/a |
| 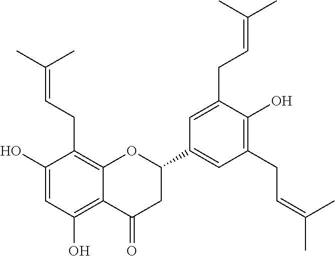 [SMILES: CC(C)=CCc1cc([C@@H]2CC(=O)c3c(O)cc(O)c(CC=C(C)C)c3O2)cc(CC=C(C)C)c1O] | 5- Hydroxysophoranone | + | n/a |
| 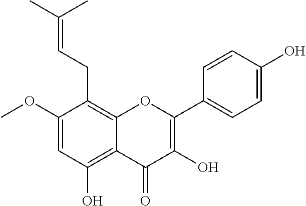 [SMILES: COc1cc(O)c2c(=O)c(O)c(-c3ccc(O)cc3)oc2c1CC=C(C)C] | Isoanhydroicaritin | + | n/a |
| 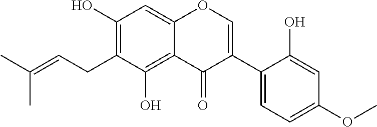 [SMILES: COc1ccc(-c2coc3cc(O)c(CC=C(C)C)c(O)c3c2=O)c(O)c1] | Gancaonin N | + | n/a |
| 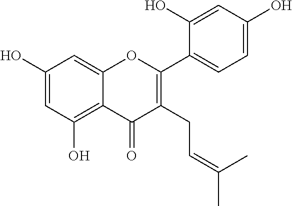 [SMILES: CC(C)=CCc1c(-c2ccc(O)cc2O)oc2cc(O)cc(O)c2c1=O] | Albanin A | + | n/a |
| 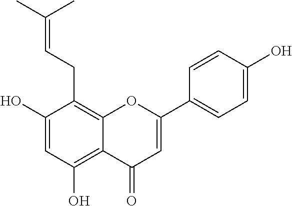 [SMILES: CC(C)=CCc1c(O)cc(O)c2c(=O)cc(-c3ccc(O)cc3)oc12] | Licoflavone C | + | n/a |
| 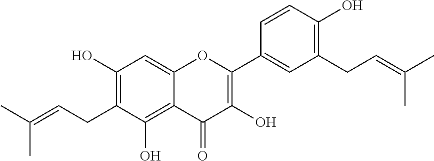 [SMILES: CC(C)=CCc1cc(-c2oc3cc(O)c(CC=C(C)C)c(O)c3c(=O)c2O)ccc1O] | Glyasperin A | + | n/a |
| 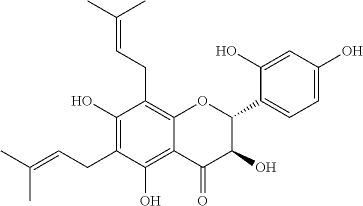 [SMILES: CC(C)=CCc1c(O)c(CC=C(C)C)c2c(c1O)C(=O)[C@H](O)[C@@H](c1ccc(O)cc1O)O2] | Kushenol L | + | n/a |
| 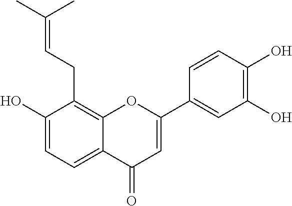 [SMILES: CC(C)=CCc1c(O)ccc2c(=O)cc(-c3ccc(O)c(O)c3)oc12] | Corylifol C | + | n/a |
| 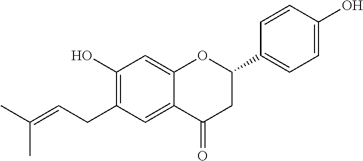 [SMILES: CC(C)=CCc1cc2c(cc1O)O[C@H](c1ccc(O)cc1)CC2=O] | Bavachin | + | n/a |
| 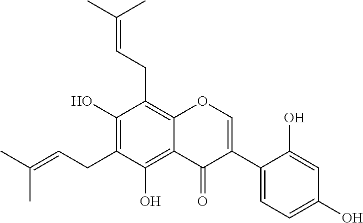 [SMILES: CC(C)=CCc1c(O)c(CC=C(C)C)c2occ(-c3ccc(O)cc3O)c(=O)c2c1O] | 8-Prenylluteone | + | n/a |
| 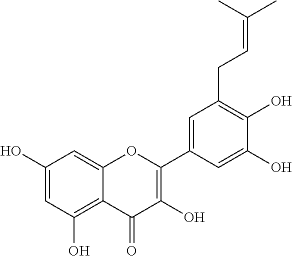 [SMILES: CC(C)=CCc1cc(-c2oc3cc(O)cc(O)c3c(=O)c2O)cc(O)c1O] | Uralenol | + | n/a |
| 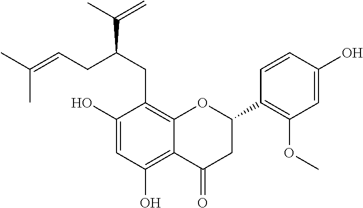 [SMILES: C=C(C)[C@H](CC=C(C)C)Cc1c(O)cc(O)c2c1O[C@H](c1ccc(O)cc1OC)CC2=O] | Leachianone A | + | n/a |
| 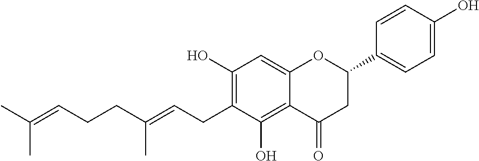 [SMILES: CC(C)=CCC/C(C)=C/Cc1c(O)cc2c(c1O)C(=O)C[C@@H](c1ccc(O)cc1)O2] | 6-Geranylnaringenin | + | n/a |
| 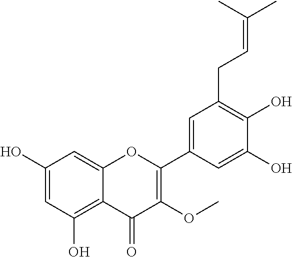 [SMILES: COc1c(-c2cc(O)c(O)c(CC=C(C)C)c2)oc2cc(O)cc(O)c2c1=O] | Uralenol-3- methylether | + | n/a |
| 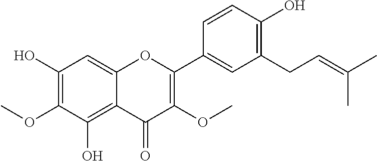 [SMILES: COc1c(O)cc2oc(-c3ccc(O)c(CC=C(C)C)c3)c(OC)c(=O)c2c1O] | 5,7,4-Trihydroxy-3,6- dimethoxy-3- prenylflavone | + | n/a |
| 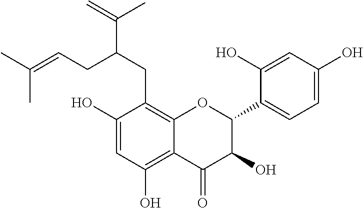 [SMILES: C=C(C)C(CC=C(C)C)Cc1c(O)cc(O)c2c1O[C@H](c1ccc(O)cc1O)[C@@H](O)C2=O] | Kushenol X | + | n/a |
| 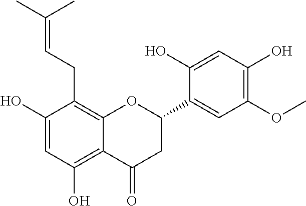 [SMILES: COc1cc([C@@H]2CC(=O)c3c(O)cc(O)c(CC=C(C)C)c3O2)c(O)cc1O] | Kushenol W | + | n/a |
| 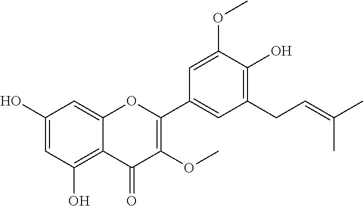 [SMILES: COc1cc(-c2oc3cc(O)cc(O)c3c(=O)c2OC)cc(CC=C(C)C)c1O] | Dodoviscin J | + | n/a |
| 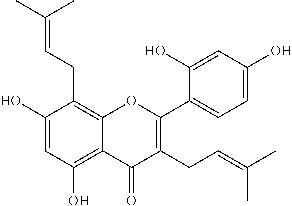 [SMILES: CC(C)=CCc1c(-c2ccc(O)cc2O)oc2c(CC=C(C)C)c(O)cc(O)c2c1=O] | Mulberrin | + | n/a |
| 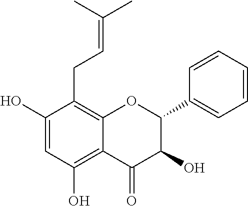 [SMILES: CC(C)=CCc1c(O)cc(O)c2c1O[C@H](c1ccccc1)[C@@H](O)C2=O] | Glepidotin B | + | n/a |
| 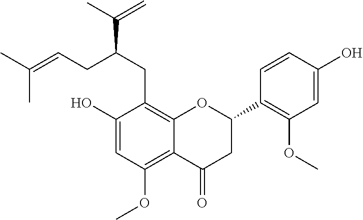 [SMILES: C=C(C)[C@H](CC=C(C)C)Cc1c(O)cc(OC)c2c1O[C@H](c1ccc(O)cc1OC)CC2=O] | 2′-methoxykurarinone | + | n/a |
| 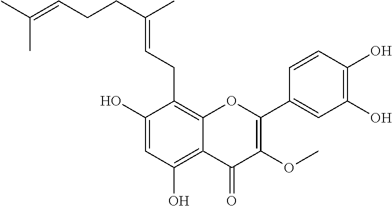 [SMILES: COc1c(-c2ccc(O)c(O)c2)oc2c(C/C=C(\C)CCC=C(C)C)c(O)cc(O)c2c1=O] | 5,7,3′,4′- Tetrahydroxy-3- methoxy-8- geranylflavone | + | n/a |
| 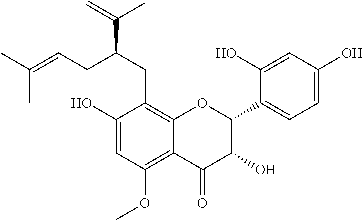 [SMILES: C=C(C)[C@H](CC=C(C)C)Cc1c(O)cc(OC)c2c1O[C@H](c1ccc(O)cc1O)[C@H](O)C2=O] | Kushenol N | + | n/a |
| 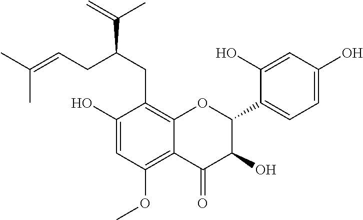 [SMILES: C=C(C)[C@H](CC=C(C)C)Cc1c(O)cc(OC)c2c1O[C@H](c1ccc(O)cc1O)[C@@H](O)C2=O] | Kushenol I | + | n/a |
| 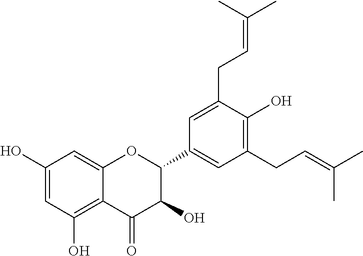 [SMILES: CC(C)=CCc1cc([C@H]2Oc3cc(O)cc(O)c3C(=O)[C@@H]2O)cc(CC=C(C)C)c1O] | Cathayanon H | + | n/a |
| 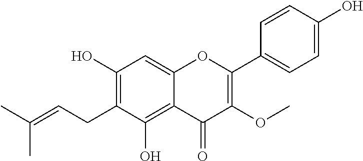 [SMILES: COc1c(-c2ccc(O)cc2)oc2cc(O)c(CC=C(C)C)c(O)c2c1=O] | Topazolin | + | n/a |
| 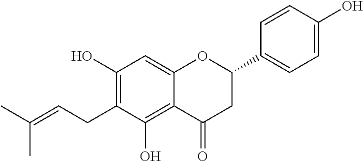 [SMILES: CC(C)=CCc1c(O)cc2c(c1O)C(=O)C[C@@H](c1ccc(O)cc1)O2] | 6-Prenylnaringenin | + | n/a |
| 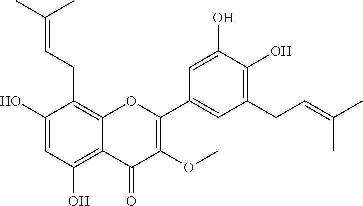 [SMILES: COc1c(-c2cc(O)c(O)c(CC=C(C)C)c2)oc2c(CC=C(C)C)c(O)cc(O)c2c1=O] | 5,7,3′,4′- Tetrahydroxy-3- methoxy-8,5′- diprenylflavone | + | n/a |
| 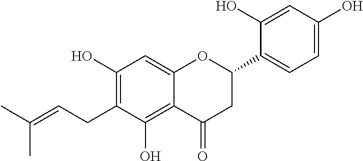 [SMILES: CC(C)=CCc1c(O)cc2c(c1O)C(=O)C[C@@H](c1ccc(O)cc1O)O2] | Cudraflavanone B | + | n/a |
| 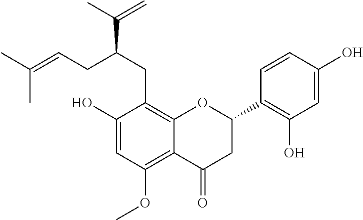 [SMILES: C=C(C)[C@H](CC=C(C)C)Cc1c(O)cc(OC)c2c1O[C@H](c1ccc(O)cc1O)CC2=O] | Kurarinone | + | n/a |
| 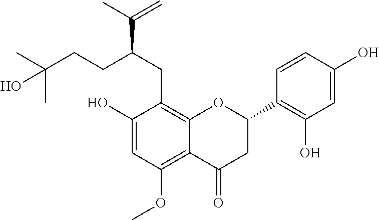 [SMILES: C=C(C)[C@H](CCC(C)(C)O)Cc1c(O)cc(OC)c2c1O[C@H](c1ccc(O)cc1O)CC2=O] | Kurarinol | + | n/a |
| 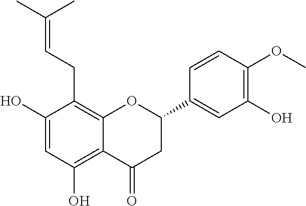 [SMILES: COc1ccc([C@@H]2CC(=O)c3c(O)cc(O)c(CC=C(C)C)c3O2)cc1O] | 5,7,3′-Trihydroxy-4′- methoxy-8- prenylflavanone | + | n/a |
| 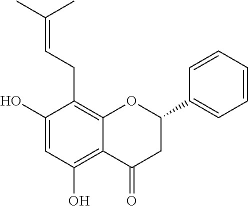 [SMILES: CC(C)=CCc1c(O)cc(O)c2c1O[C@H](c1ccccc1)CC2=O] | Glabranin | + | n/a |
| 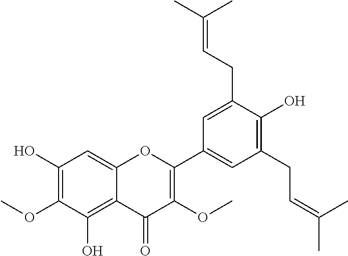 [SMILES: COc1c(O)cc2oc(-c3cc(CC=C(C)C)c(O)c(CC=C(C)C)c3)c(OC)c(=O)c2c1O] | 5,7,4′-Trihydroxy-3,6- dimethoxy-3′,5′- diprenylflavone | + | n/a |
| 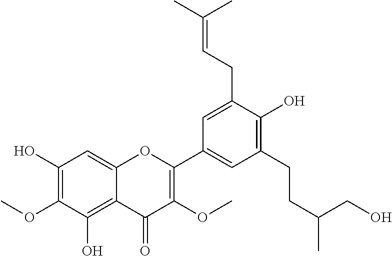 [SMILES: COc1c(O)cc2oc(-c3cc(CC=C(C)C)c(O)c(CCC(C)CO)c3)c(OC)c(=O)c2c1O] | 5′-Prenylaliarin | + | n/a |
| 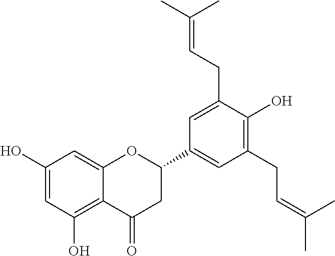 [SMILES: CC(C)=CCc1cc([C@@H]2CC(=O)c3c(O)cc(O)cc3O2)cc(CC=C(C)C)c1O] | abyssinone V | + | n/a |
| 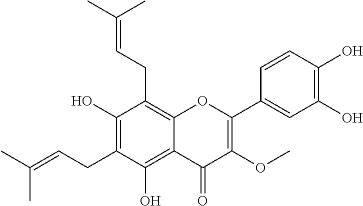 [SMILES: COc1c(-c2ccc(O)c(O)c2)oc2c(CC=C(C)C)c(O)c(CC=C(C)C)c(O)c2c1=O] | broussoflavonol B | + | n/a |
| 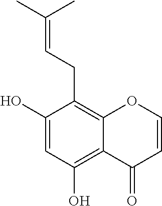 [SMILES: CC(C)=CCc1c(O)cc(O)c2c(=O)ccoc12] | Eriosematin A | n/a | n/a |
| 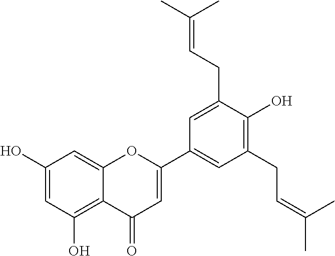 [SMILES: CC(C)=CCc1cc(-c2cc(=O)c3c(O)cc(O)cc3o2)cc(CC=C(C)C)c1O] | Honyucitrin | n/a | n/a |

TABLE 1b

| Structure | Name | activity | IC₅₀ |
|---|---|---|---|
| 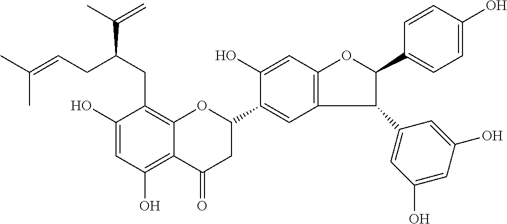 | Sophoraflavanone I | ++ | n/a |
| 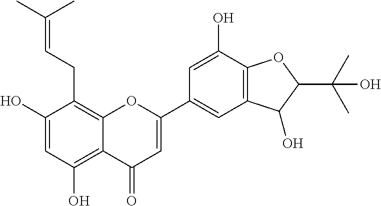 [SMILES: CC(C)=CCc1c(O)cc(O)c2c(=O)cc(-c3cc(O)c4c(c3)C(O)C(C(C)(C)O)O4)oc12] | Epimedokoreanin C | + | n/a |
| 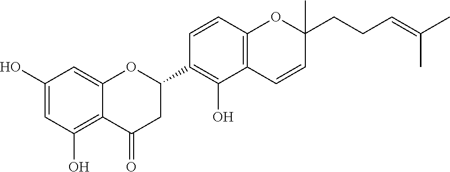 [SMILES: CC(C)=CCCC1(C)C=Cc2c(ccc([C@@H]3CC(=O)c4c(O)cc(O)cc4O3)c2O)O1] | Sanggenon N | + | n/a |
| 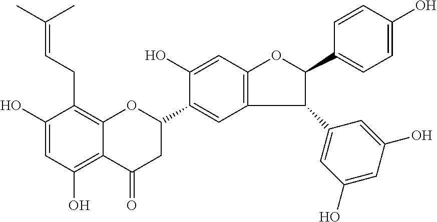 | Sophoraflavanone H | + | n/a |
| 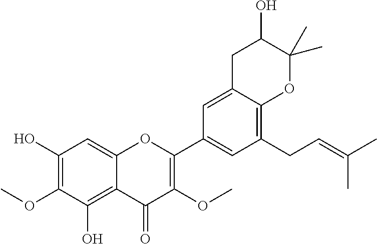 [SMILES: COc1c(O)cc2oc(-c3cc(CC=C(C)C)c4c(c3)CC(O)C(C)(C)O4)c(OC)c(=O)c2c1O] | Dodovisone A | + | n/a |
| 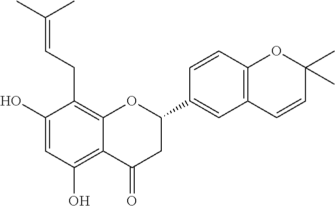 [SMILES: CC(C)=CCc1c(O)cc(O)c2c1O[C@H](c1ccc3c(c1)C=CC(C)(C)O3)CC2=O] | Euchrenone A10 | + | n/a |
| 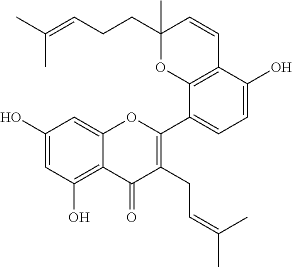 [SMILES: CC(C)=CCCC1(C)C=Cc2c(O)ccc(-c3oc4cc(O)cc(O)c4c(=O)c3CC=C(C)C)c2O1] | Sanggenone K | + | n/a |

TABLE 1c

| Structure | Name | activity | IC₅₀ |
|---|---|---|---|
| 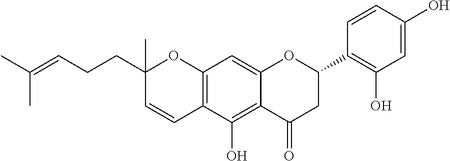 [SMILES: CC(C)=CCCC1(C)C=Cc2c(cc3c(c2O)C(=O)C[C@@H](c2ccc(O)cc2O)O3)O1] | Kuwanol C | ++ | n/a |
| 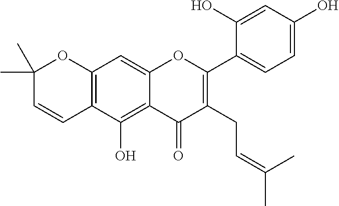 [SMILES: CC(C)=CCc1c(-c2ccc(O)cc2O)oc2cc3c(c(O)c2c1=O)C=CC(C)(C)O3] | Cudraflavone B | ++ | n/a |
| 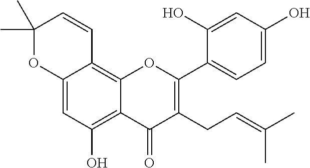 [SMILES: CC(C)=CCc1c(-c2ccc(O)cc2O)oc2c3c(cc(O)c2c1=O)OC(C)(C)C=C3] | Morusin | ++ | n/a |
| 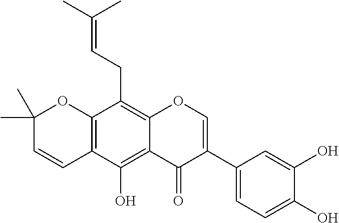 [SMILES: CC(C)=CCc1c2c(c(O)c3c(=O)c(-c4ccc(O)c(O)c4)coc13)C=CC(C)(C)O2] | Auriculasin | + | n/a |
| 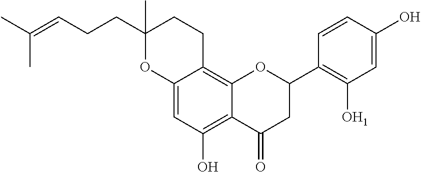 [SMILES: CC(C)=CCCC1(C)CCc2c(cc(O)c3c2OC(c2ccc(O)cc2O)CC3=O)O1] | Sanggenol L | + | n/a |

TABLE 1d

| Structure | Name | activity | IC₅₀ |
|---|---|---|---|
| 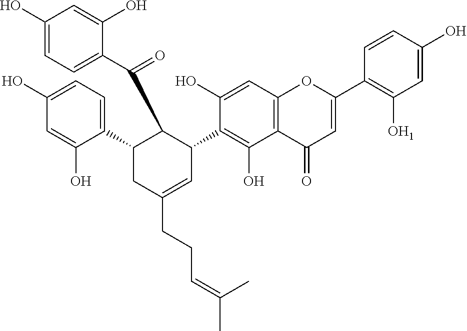 [SMILES: CC(C)=CCCC1=C[C@@H](c2c(O)cc3oc(-c4ccc(O)cc4O)cc(=O)c3c2O)[C@H](C(=O)c2ccc(O)cc2O)[C@@H](c2ccc(O)cc2O)C1] | Multicaulisin | +++ | 17 |
| 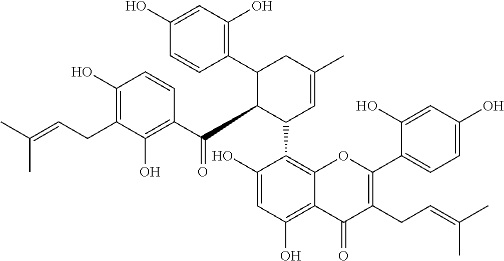 | Kuwanon H | ++ | n/a |

TABLE 1e

| Structure | Name | activity | IC₅₀ |
|---|---|---|---|
| 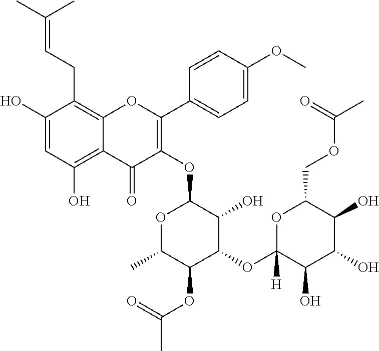 [SMILES: COc1ccc(-c2oc3c(CC=C(C)C)c(O)cc(O)c3c(=O)c2O[C@@H]2O[C@@H](C)[C@H](OC(C)=O)[C@@H](O[C@@H]3O[C@H](COC(C)=O)[C@@H](O)[C@H](O)[C@H]3O)[C@H]2O)cc1] | Korepimedoside A | ++ | n/a |
| 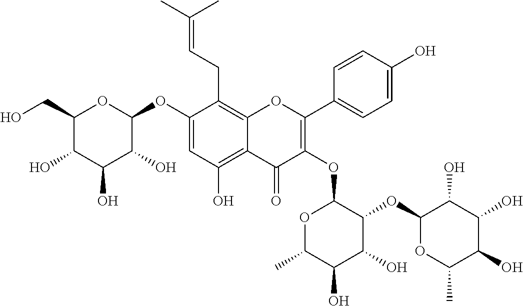 [SMILES: CC(C)=CCc1c(O[C@@H]2O[C@H](CO)[C@@H](O)[C@H](O)[C@H]2O)cc(O)c2c(=O)c(O[C@@H]3O[C@@H](C)[C@H](O)[C@@H](O)[C@H]3O[C@@H]3O[C@@H](C)[C@H](O)[C@@H](O)[C@H]3O)c(-c3ccc(O)cc3)oc12] | Baohuoside V | ++ | n/a |
| 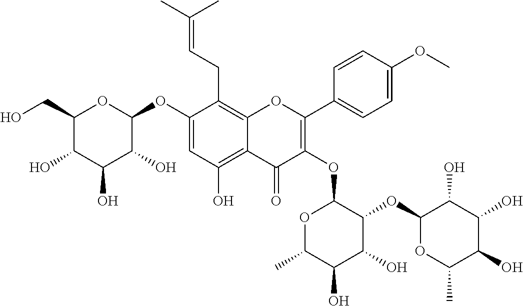 [SMILES: COc1ccc(-c2oc3c(CC=C(C)C)c(O[C@@H]4O[C@H](CO)[C@@H](O)[C@H](O)[C@H]4O)cc(O)c3c(=O)c2O[C@@H]2O[C@@H](C)[C@H](O)[C@@H](O)[C@H]2O[C@@H]2O[C@@H](C)[C@H](O)[C@@H](O)[C@H]2O)cc1] | Epimedin C | ++ | n/a |
| 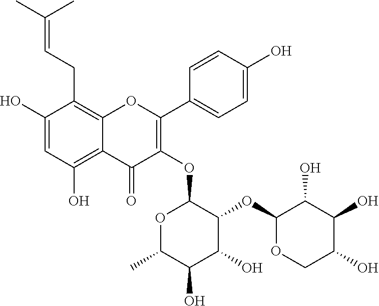 [SMILES: CC(C)=CCc1c(O)cc(O)c2c(=O)c(O[C@@H]3O[C@@H](C)[C@H](O)[C@@H](O)[C@H]3O[C@@H]3OC[C@@H](O)[C@H](O)[C@H]3O)c(-c3ccc(O)cc3)oc12] | Ikarisoside F | ++ | n/a |
| 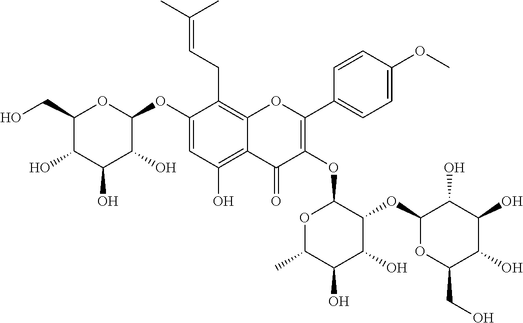 [SMILES: COc1ccc(-c2oc3c(CC=C(C)C)c(O[C@@H]4O[C@H](CO)[C@@H](O)[C@H](O)[C@H]4O)cc(O)c3c(=O)c2O[C@@H]2O[C@@H](C)[C@H](O)[C@@H](O)[C@H]2O[C@@H]2O[C@H](CO)[C@@H](O)[C@H](O)[C@H]2O)cc1] | Epimedin A | ++ | n/a |
| 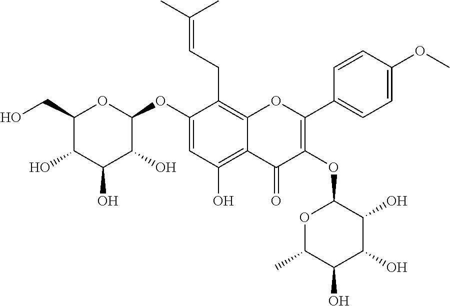 [SMILES: COc1ccc(-c2oc3c(CC=C(C)C)c(O[C@@H]4O[C@H](CO)[C@@H](O)[C@H](O)[C@H]4O)cc(O)c3c(=O)c2O[C@@H]2O[C@@H](C)[C@H](O)[C@@H](O)[C@H]2O)cc1] | Icariin | ++ | n/a |
| 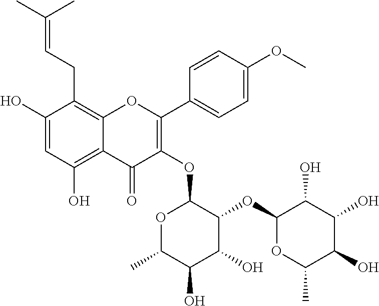 [SMILES: COc1ccc(-c2oc3c(CC=C(C)C)c(O)cc(O)c3c(=O)c2O[C@@H]2O[C@@H](C)[C@H](O)[C@@H](O)[C@H]2O[C@@H]2O[C@@H](C)[C@H](O)[C@@H](O)[C@H]2O)cc1] | 2″-O- Rhamnosylicariside II | ++ | n/a |
| 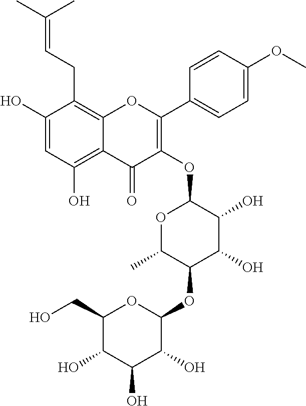 [SMILES: COc1ccc(-c2oc3c(CC=C(C)C)c(O)cc(O)c3c(=O)c2O[C@@H]2O[C@@H](C)[C@H](O[C@@H]3O[C@H](CO)[C@@H](O)[C@H](O)[C@H]3O)[C@@H](O)[C@H]2O)cc1] | Baohuoside VII | ++ | n/a |
| 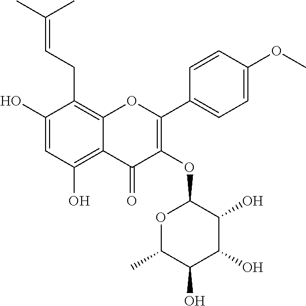 [SMILES: COc1ccc(-c2oc3c(CC=C(C)C)c(O)cc(O)c3c(=O)c2O[C@@H]2O[C@@H](C)[C@H](O)[C@@H](O)[C@H]2O)cc1] | Baohuoside I | ++ | n/a |
| 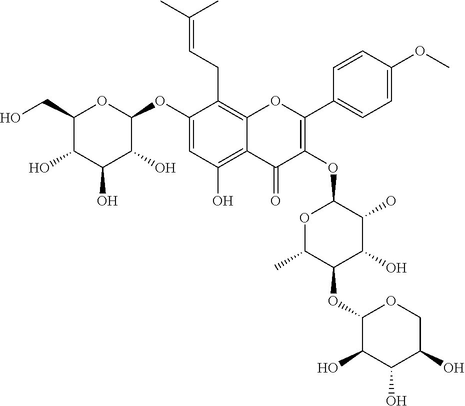 [SMILES: COc1ccc(-c2oc3c(CC=C(C)C)c(O[C@@H]4O[C@H](CO)[C@@H](O)[C@H](O)[C@H]4O)cc(O)c3c(=O)c2O[C@@H]2O[C@@H](C)[C@H](O[C@@H]3OC[C@@H](O)[C@H](O)[C@H]3O)[C@@H](O)[C@H]2O)cc1] | Epimedin B1 | ++ | n/a |
| 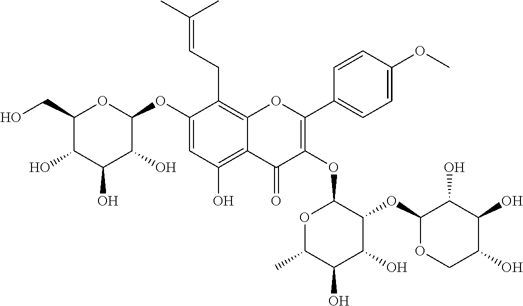 [SMILES: COc1ccc(-c2oc3c(CC=C(C)C)c(O[C@@H]4O[C@H](CO)[C@@H](O)[C@H](O)[C@H]4O)cc(O)c3c(=O)c2O[C@@H]2O[C@@H](C)[C@H](O)[C@@H](O)[C@H]2O[C@@H]2OC[C@@H](O)[C@H](O)[C@H]2O)cc1] | Epimedin B | ++ | n/a |
| 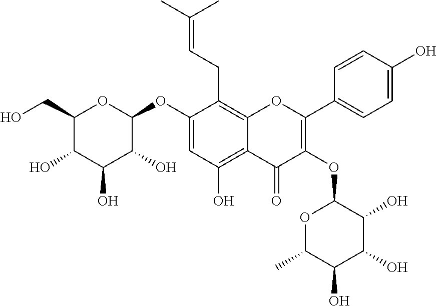 [SMILES: CC(C)=CCc1c(O[C@@H]2O[C@H](CO)[C@@H](O)[C@H](O)[C@H]2O)cc(O)c2c(=O)c(O[C@@H]3O[C@@H](C)[C@H](O)[C@@H](O)[C@H]3O)c(-c3ccc(O)cc3)oc12] | Epimedoside A | + | n/a |
| 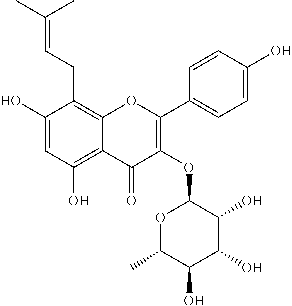 [SMILES: CC(C)=CCc1c(O)cc(O)c2c(=O)c(O[C@@H]3O[C@@H](C)[C@H](O)[C@@H](O)[C@H]3O)c(-c3ccc(O)cc3)oc12] | Baohuoside II | + | n/a |
| 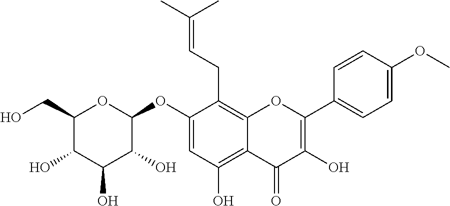 [SMILES: COc1ccc(-c2oc3c(CC=C(C)C)c(O[C@@H]4O[C@H](CO)[C@@H](O)[C@H](O)[C@H]4O)cc(O)c3c(=O)c2O)cc1] | Icariside I | + | n/a |
| 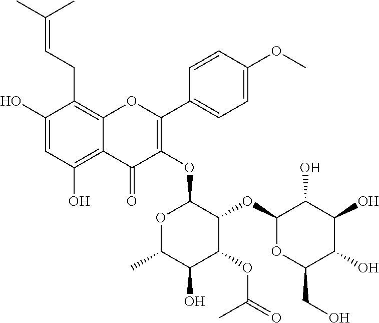 [SMILES: COc1ccc(-c2oc3c(CC=C(C)C)c(O)cc(O)c3c(=O)c2O[C@@H]2O[C@@H](C)[C@H](O)[C@@H](OC(C)=O)[C@H]2O[C@@H]2O[C@H](CO)[C@@H](O)[C@H](O)[C@H]2O)cc1] | Sagittatoside C | + | n/a |
| 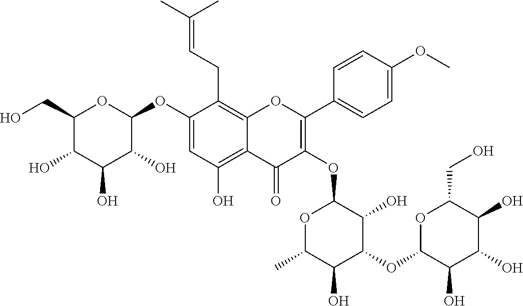 [SMILES: COc1ccc(-c2oc3c(CC=C(C)C)c(O[C@@H]4O[C@H](CO)[C@@H](O)[C@H](O)[C@H]4O)cc(O)c3c(=O)c2O[C@@H]2O[C@@H](C)[C@H](O)[C@@H](O[C@@H]3O[C@H](CO)[C@@H](O)[C@H](O)[C@H]3O)[C@H]2O)cc1] | Epimedin A1 | + | n/a |
| 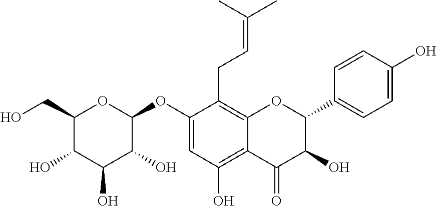 [SMILES: CC(C)=CCc1c(O[C@@H]2O[C@H](CO)[C@@H](O)[C@H](O)[C@H]2O)cc(O)c2c1O[C@H](c1ccc(O)cc1)[C@@H](O)C2=O] | Phellamurin | + | n/a |
| 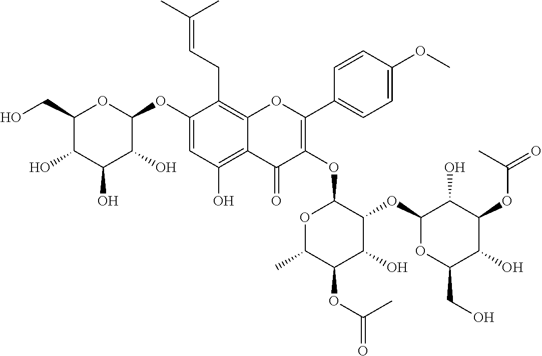 [SMILES: COc1ccc(-c2oc3c(CC=C(C)C)c(O[C@@H]4O[C@H](CO)[C@@H](O)[C@H](O)[C@H]4O)cc(O)c3c(=O)c2O[C@@H]2O[C@@H](C)[C@H](OC(C)=O)[C@@H](O)[C@H]2O[C@@H]2O[C@H](CO)[C@@H](O)[C@H](OC(C)=O)[C@H]2O)cc1] | Caohuoside E | + | n/a |
| 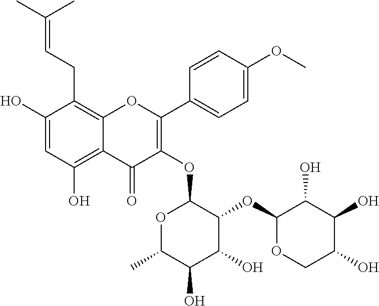 [SMILES: COc1ccc(-c2oc3c(CC=C(C)C)c(O)cc(O)c3c(=O)c2O[C@@H]2O[C@@H](C)[C@H](O)[C@@H](O)[C@H]2O[C@@H]2OC[C@@H](O)[C@H](O)[C@H]2O)cc1] | Sagittatoside B | + | n/a |
| 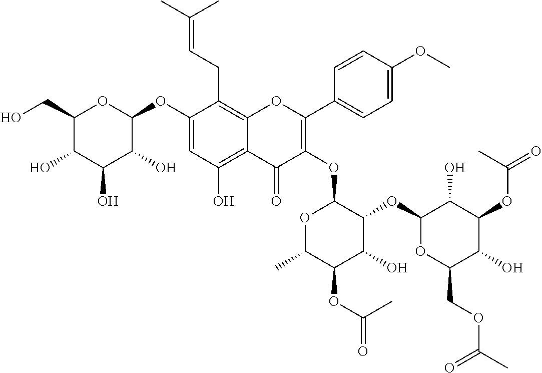 [SMILES: COc1ccc(-c2oc3c(CC=C(C)C)c(O[C@@H]4O[C@H](CO)[C@@H](O)[C@H](O)[C@H]4O)cc(O)c3c(=O)c2O[C@@H]2O[C@@H](C)[C@H](OC(C)=O)[C@@H](O)[C@H]2O[C@@H]2O[C@H](COC(C)=O)[C@@H](O)[C@H](OC(C)=O)[C@H]2O)cc1] | Epimedin K | + | n/a |
| 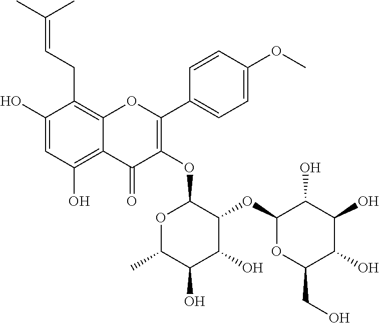 [SMILES: COc1ccc(-c2oc3c(CC=C(C)C)c(O)cc(O)c3c(=O)c2O[C@@H]2O[C@@H](C)[C@H](O)[C@@H](O)[C@H]2O[C@@H]2O[C@H](CO)[C@@H](O)[C@H](O)[C@H]2O)cc1] | Sagittatoside A | + | n/a |
| 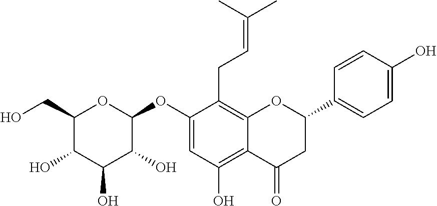 [SMILES: CC(C)=CCc1c(O[C@@H]2O[C@H](CO)[C@@H](O)[C@H](O)[C@H]2O)cc(O)c2c1O[C@H](c1ccc(O)cc1)CC2=O] | Flavaprin | + | n/a |

TABLE 1f

| Structure | Name | activity | IC₅₀ |
|---|---|---|---|
| 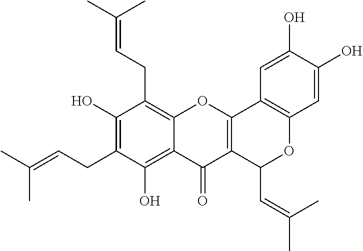 [SMILES: CC(C)=CCc1c(O)c(CC=C(C)C)c2oc3c(c(=O)c2c1O)C(C=C(C)C)Oc1cc(O)c(O)cc1-3] | Artoheterophyllin B | + | n/a |
| 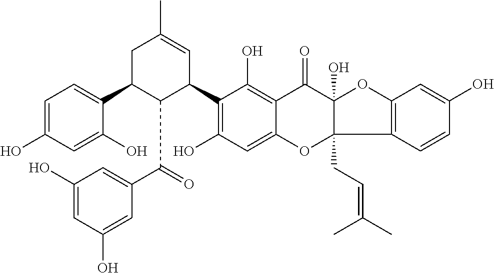 [SMILES: CC(C)=CC[C@]12Oc3cc(O)c([C@@H]4C=C(C)C[C@H](c5ccc(O)cc5O)[C@H]4C(=O)c4cc(O)cc(O)c4)c(O)c3C(=O)[C@@]1(O)Oc1cc(O)ccc12] | Sanggenone D | +++ | n/a |
| 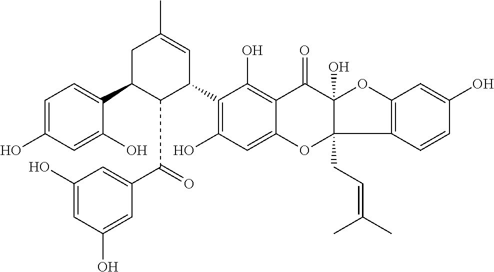 [SMILES: CC(C)=CC[C@]12Oc3cc(O)c([C@H]4C=C(C)C[C@H](c5ccc(O)cc5O)[C@H]4C(=O)c4cc(O)cc(O)c4)c(O)c3C(=O)[C@@]1(O)Oc1cc(O)ccc12] | Sanggenone C | +++ | n/a |
| 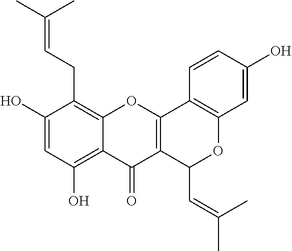 [SMILES: CC(C)=CCc1c(O)cc(O)c2c(=O)c3c(oc12)-c1ccc(O)cc1OC3C=C(C)C] | Cyclomulberrin | ++ | n/a |
| 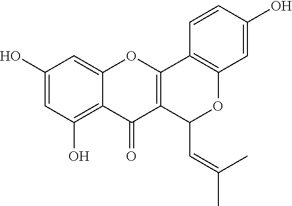 [SMILES: CC(C)=CC1Oc2cc(O)ccc2-c2oc3cc(O)cc(O)c3c(=O)c21] | Cyclocommunol | + | n/a |
| 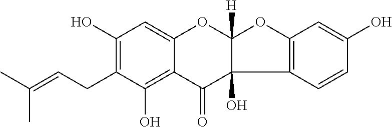 [SMILES: CC(C)=CCc1c(O)cc2c(c1O)C(=O)[C@@]1(O)c3ccc(O)cc3O[C@H]1O2] | Lupinol C | + | n/a |
| 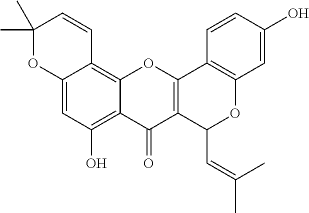 [SMILES: CC(C)=CC1Oc2cc(O)ccc2-c2oc3c4c(cc(O)c3c(=O)c21)OC(C)(C)C=C4] | Cyclomorusin | + | n/a |
| 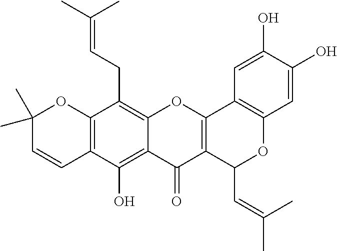 [SMILES: CC(C)=CCc1c2c(c(O)c3c(=O)c4c(oc13)-c1cc(O)c(O)cc1OC4C=C(C)C)C=CC(C)(C)O2] | Cycloheterophyllin | + | n/a |

TABLE 1g

| Structure | Name | activity | IC₅₀ |
|---|---|---|---|
| 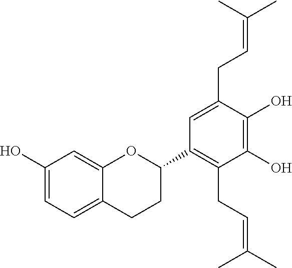 [SMILES: CC(C)=CCc1cc([C@@H]2CCc3ccc(O)cc3O2)c(CC=C(C)C)c(O)c1O] | Kazinol A | + | n/a |
| 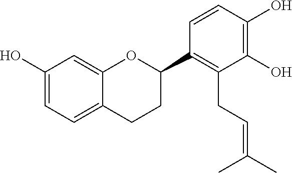 [SMILES: CC(C)=CCc1c([C@H]2CCc3ccc(O)cc3O2)ccc(O)c1O] | Kazinol U | + | n/a |
| 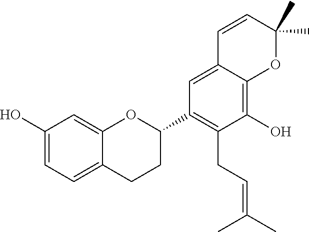 | Kazinol B | + | n/a |
| 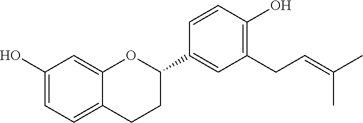 [SMILES: CC(C)=CCc1cc([C@@H]2CCc3ccc(O)cc3O2)ccc1O] | 7,4′-Dihydroxy-3′- prenylflavan | n/a | n/a |
| 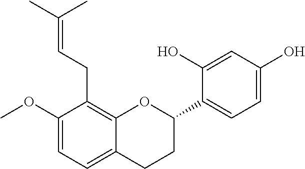 [SMILES: COc1ccc2c(c1CC=C(C)C)O[C@H](c1ccc(O)cc1O)CC2] | 2′,4′-Dihydroxy-7- methoxy-8- prenylflavan | + | n/a |

### Example 2—prenylated isoflavones, isoflavanones, and isoflavenes

TABLE 2a

| Structure | Name | activity | IC₅₀ |
|---|---|---|---|
| 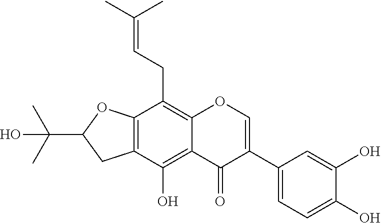 [SMILES: CC(C)=CCc1c2c(c(O)c3c(=O)c(-c4ccc(O)c(O)c4)coc13)CC(C(C)(C)O)O2] | furowanin A | ++ | 7 |
| 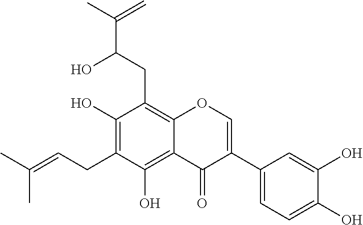 [SMILES: C=C(C)C(O)Cc1c(O)c(CC=C(C)C)c(O)c2c(=O)c(-c3ccc(O)c(O)c3)coc12] | millewanin H | ++ | n/a |
| 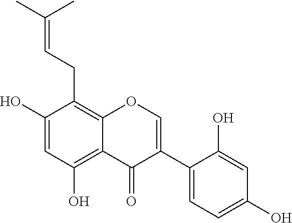 [SMILES: CC(C)=CCc1c(O)cc(O)c2c(=O)c(-c3ccc(O)cc3O)coc12] | 2,3-dehydrokievitone | ++ | n/a |
| 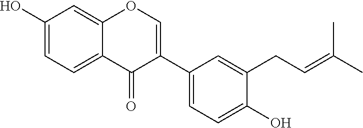 [SMILES: CC(C)=CCc1cc(-c2coc3cc(O)ccc3c2=O)ccc1O] | Neobavaisoflavone | ++ | n/a |
| 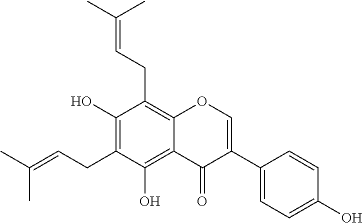 [SMILES: CC(C)=CCc1c(O)c(CC=C(C)C)c2occ(-c3ccc(O)cc3)c(=O)c2c1O] | 6,8-Diprenylgenistein | ++ | n/a |
| 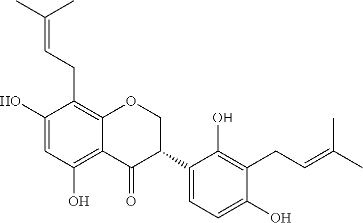 [SMILES: CC(C)=CCc1c(O)ccc([C@H]2COc3c(CC=C(C)C)c(O)cc(O)c3C2=O)c1O] | 5,7,2′,4′- Tetrahydroxy-8,3′- di(gamma,gamma- dimethylallyl)- isoflavanone | ++ | n/a |
| 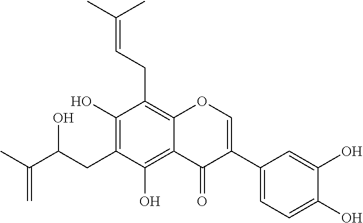 [SMILES: C=C(C)C(O)Cc1c(O)c(CC=C(C)C)c2occ(-c3ccc(O)c(O)c3)c(=O)c2c1O] | millewanin G | + | n/a |
| 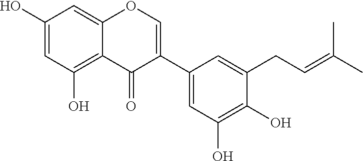 [SMILES: CC(C)=CCc1cc(-c2coc3cc(O)cc(O)c3c2=O)cc(O)c1O] | glycyrrhisoflavone | + | n/a |
| 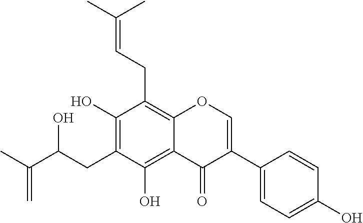 [SMILES: C=C(C)C(O)Cc1c(O)c(CC=C(C)C)c2occ(-c3ccc(O)cc3)c(=O)c2c1O] | Isoerysenegalensein E | + | n/a |
| 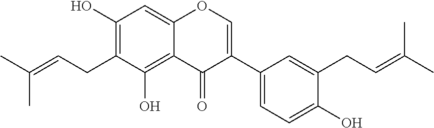 [SMILES: CC(C)=CCc1cc(-c2coc3cc(O)c(CC=C(C)C)c(O)c3c2=O)ccc1O] | Lupalbigenin | + | n/a |
| 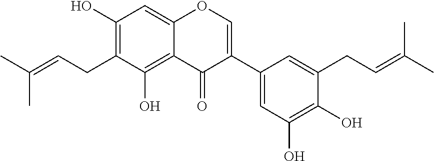 [SMILES: CC(C)=CCc1cc(-c2coc3cc(O)c(CC=C(C)C)c(O)c3c2=O)cc(O)c1O] | Imbricataflavone A | + | n/a |
| 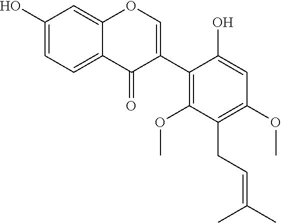 [SMILES: COc1cc(O)c(-c2coc3cc(O)ccc3c2=O)c(OC)c1CC=C(C)C] | Licoricone | + | n/a |
| 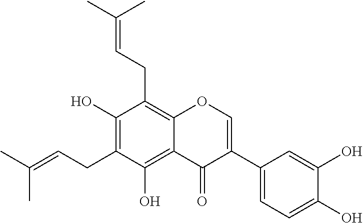 [SMILES: CC(C)=CCc1c(O)c(CC=C(C)C)c2occ(-c3ccc(O)c(O)c3)c(=O)c2c1O] | 6,8-Diprenylorobol | + | n/a |
| 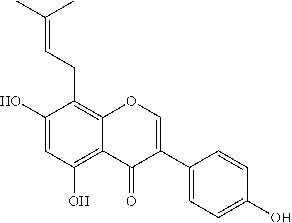 [SMILES: CC(C)=CCc1c(O)cc(O)c2c(=O)c(-c3ccc(O)cc3)coc12] | Lupiwighteone | + | n/a |
| 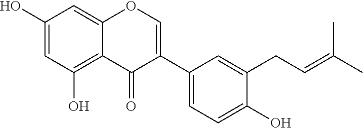 [SMILES: CC(C)=CCc1cc(-c2coc3cc(O)cc(O)c3c2=O)ccc1O] | Isowighteone | + | n/a |
| 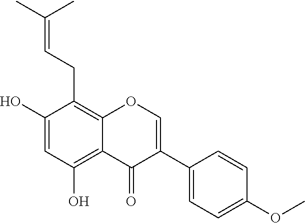 [SMILES: COc1ccc(-c2coc3c(CC=C(C)C)c(O)cc(O)c3c2=O)cc1] | Gancaonin M | + | n/a |
| 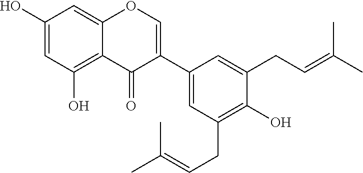 [SMILES: CC(C)=CCc1cc(-c2coc3cc(O)cc(O)c3c2=O)cc(CC=C(C)C)c1O] | 3′,5′-Diprenylgenistein | + | n/a |
| 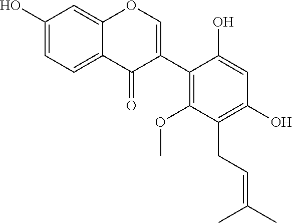 [SMILES: COc1c(CC=C(C)C)c(O)cc(O)c1-c1coc2cc(O)ccc2c1=O] | Glicoricone | + | n/a |
| 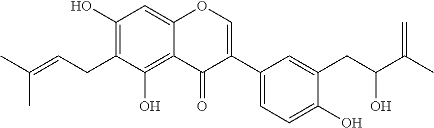 [SMILES: C=C(C)C(O)Cc1cc(-c2coc3cc(O)c(CC=C(C)C)c(O)c3c2=O)ccc1O] | Derrisisoflavone B | + | n/a |
| 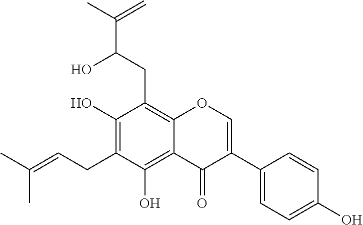 [SMILES: C=C(C)C(O)Cc1c(O)c(CC=C(C)C)c(O)c2c(=O)c(-c3ccc(O)cc3)coc12] | Erysenegalensein E | + | n/a |
| 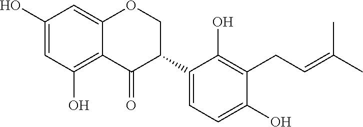 [SMILES: CC(C)=CCc1c(O)ccc([C@H]2COc3cc(O)cc(O)c3C2=O)c1O] | Dihydrolicoisoflavone | + | n/a |
| 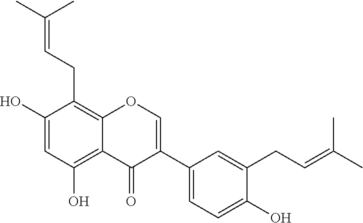 [SMILES: CC(C)=CCc1cc(-c2coc3c(CC=C(C)C)c(O)cc(O)c3c2=O)ccc1O] | Isolupalbigenin | + | n/a |
| 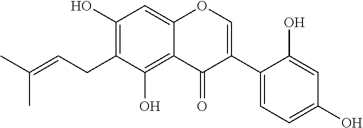 [SMILES: CC(C)=CCc1c(O)cc2occ(-c3ccc(O)cc3O)c(=O)c2c1O] | Luteone | + | n/a |
| 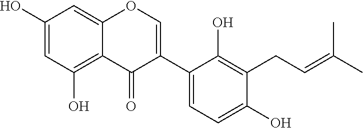 [SMILES: CC(C)=CCc1c(O)ccc(-c2coc3cc(O)cc(O)c3c2=O)c1O] | Licoisoflavone A | + | n/a |
| 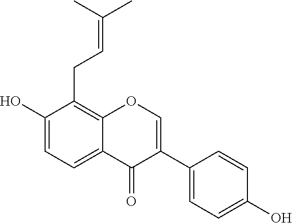 [SMILES: CC(C)=CCc1c(O)ccc2c(=O)c(-c3ccc(O)cc3)coc12] | 8-Prenyldaidzein | + | n/a |
| 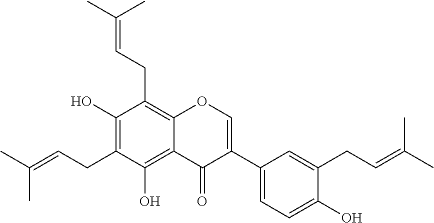 [SMILES: CC(C)=CCc1cc(-c2coc3c(CC=C(C)C)c(O)c(CC=C(C)C)c(O)c3c2=O)ccc1O] | Euchrenone B1 | + | n/a |
| 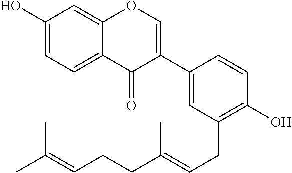 [SMILES: CC(C)=CCC/C(C)=C/Cc1cc(-c2coc3cc(O)ccc3c2=O)ccc1O] | Corylifol A | + | n/a |
| 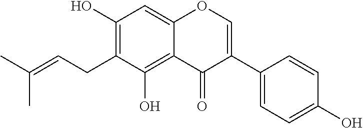 [SMILES: CC(C)=CCc1c(O)cc2occ(-c3ccc(O)cc3)c(=O)c2c1O] | Wighteone | + | n/a |
| 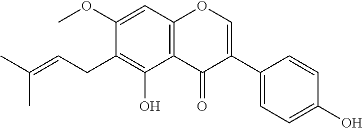 [SMILES: COc1cc2occ(-c3ccc(O)cc3)c(=O)c2c(O)c1CC=C(C)C] | Gancaonin G | + | n/a |

TABLE 2b

| Structure | Name | activity | IC₅₀ |
|---|---|---|---|
| 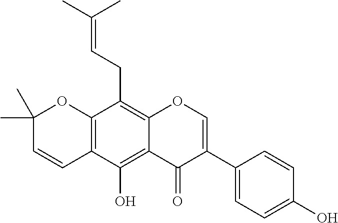 [SMILES: CC(C)=CCc1c2c(c(O)c3c(=O)c(-c4ccc(O)cc4)coc13)C=CC(C)(C)O2] | warangalone | ++ | n/a |
| 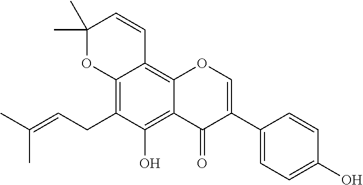 [SMILES: CC(C)=CCc1c2c(c3occ(-c4ccc(O)cc4)c(=O)c3c1O)C=CC(C)(C)O2] | Osajin | + | n/a |

TABLE 2c

| | | % | |
|---|---|---|---|
| Structure | Name | activity | IC₅₀ |
| 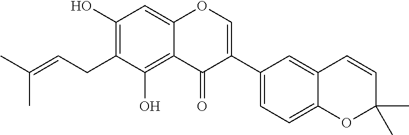 [SMILES: CC(C)=CCc1c(O)cc2occ(-c3ccc4c(c3)C=CC(C)(C)O4)c(=O)c2c1O] | Isochandalone | + | n/a |

TABLE 2d

| Structure | Name | activity | IC₅₀ |
|---|---|---|---|
| 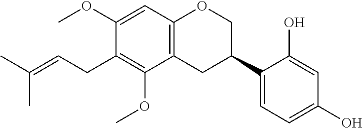 [SMILES: COc1cc2c(c(OC)c1CC=C(C)C)C[C@H](c1ccc(O)cc1O)CO2] | Glyasperin D | ++ | n/a |
| 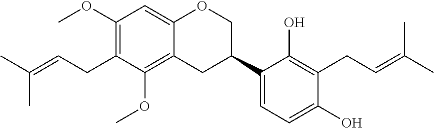 [SMILES: COc1cc2c(c(OC)c1CC=C(C)C)C[C@H](c1ccc(O)c(CC=C(C)C)c1O)CO2] | Licorisoflavan A | ++ | n/a |
| 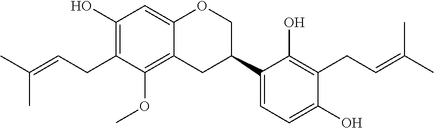 [SMILES: COc1c(CC=C(C)C)c(O)cc2c1C[C@H](c1ccc(O)c(CC=C(C)C)c1O)CO2] | Licoricidin | + | n/a |
| 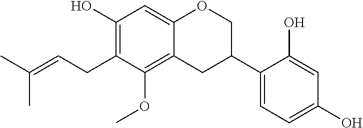 [SMILES: COc1c(CC=C(C)C)c(O)cc2c1CC(c1ccc(O)cc1O)CO2] | Glyasperin C | + | n/a |
| 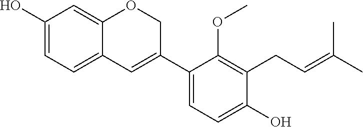 [SMILES: COc1c(C2=Cc3ccc(O)cc3OC2)ccc(O)c1CC=C(C)C] | Bidwillol A | + | n/a |
| 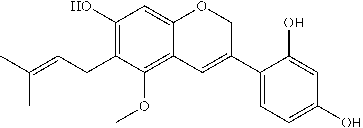 [SMILES: COc1c2c(cc(O)c1CC=C(C)C)OCC(c1ccc(O)cc1O)=C2] | Dehydroglyasperin C | + | n/a |

TABLE 2e

| Structure | Name | activity | IC₅₀ |
|---|---|---|---|
| 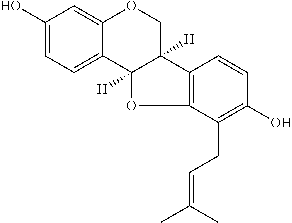 [SMILES: CC(C)=CCc1c(O)ccc2c1O[C@H]1c3ccc(O)cc3OC[C@@H]21] | Phaseollidin | + | n/a |

### Example 3—non-prenylated flavonoids and isoflavonoids

| Structure | Name | activity | IC₅₀ |
|---|---|---|---|
| 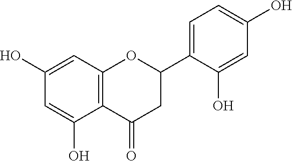 [SMILES: O=C1CC(c2ccc(O)cc2O)Oc2cc(O)cc(O)c21] | Steppogenin | + | |
| 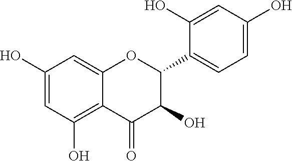 [SMILES: O=C1c2c(O)cc(O)cc2O[C@H](c2ccc(O)cc2O)[C@H]1O] | Dihydromorin | + | |
| 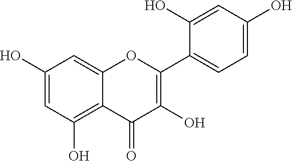 [SMILES: O=c1c(O)c(-c2ccc(O)cc2O)oc2cc(O)cc(O)c12] | morin | + | |
| 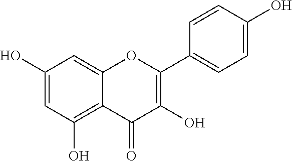 [SMILES: O=c1c(O)c(-c2ccc(O)cc2)oc2cc(O)cc(O)c12] | Kaempferol | + | |
| 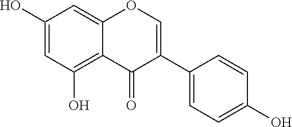 [SMILES: O=c1c(-c2ccc(O)cc2)coc2cc(O)cc(O)c12] | Genistein | + | |
| 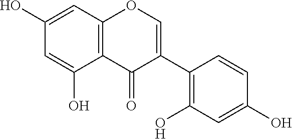 [SMILES: O=c1c(-c2ccc(O)cc2O)coc2cc(O)cc(O)c12] | 2′-hydroxygenistein | + | |
| | Biorobin | +++ | 7 |
| | Kaempferol-4′-O-beta- | ++ | 12 |
| | D-glucopyranoside | | |
| | Dracoflavan B1 | ++ | 17 |
| | Isoschaftoside | ++ | 20 |
| | 6-methoxyluteolin | ++ | 4 |
| 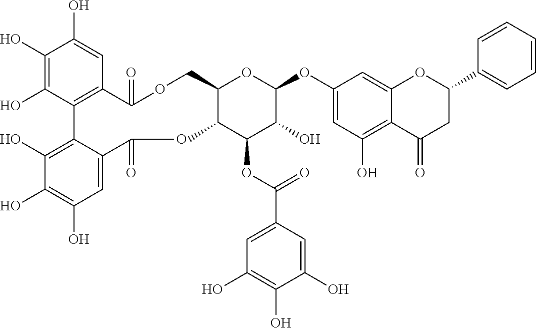 [SMILES: C[C@H]1[C@H](OC(=O)c2cc(O)c(O)c(O)c2)[C@@H](O)[C@H](Oc2cc(O)c3c(c2)O[C@H](c2ccccc2)CC3=O)O[C@@H]1COC(=O)c1cc(O)c(O)c(O)c1-c1c(C(=O)O)cc(O)c(O)c1O] | pinocembrin 7-O-(3″- galloyl-4″,6″-(S)- hexahydroxydiphenoyl)- β-D-glucose (PGHG) | +++ | 6 |
| 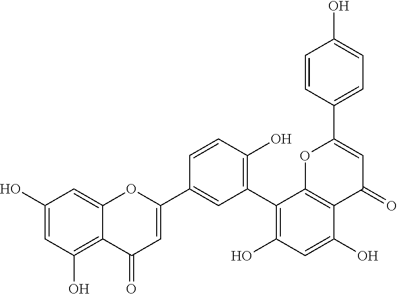 [SMILES: O=c1cc(-c2ccc(O)c(-c3c(O)cc(O)c4c(=O)cc(-c5ccc(O)cc5)oc34)c2)oc2cc(O)cc(O)c12] | Amentoflavone | ++ | 8 |
| 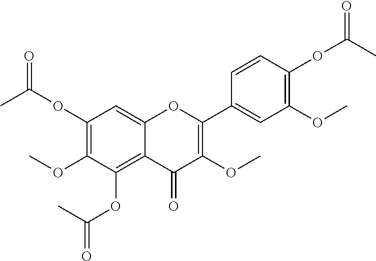 [SMILES: COc1cc(-c2oc3cc(OC(C)=O)c(OC)c(OC(C)=O)c3c(=O)c2OC)ccc1OC(C)=O] | Jaceidin triacetate | +++ | 12 |
| 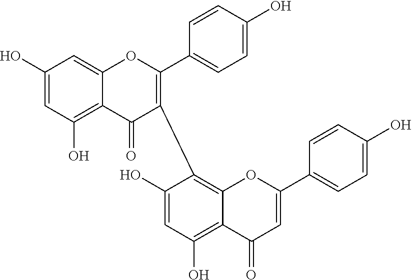 [SMILES: O=c1c(-c2c(O)cc(O)c3c(=O)cc(-c4ccc(O)cc4)oc23)c(-c2ccc(O)cc2)oc2cc(O)cc(O)c12] | 3,8′-Biapigenin | | |
| 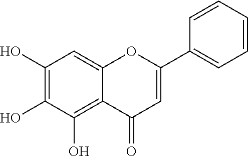 [SMILES: O=c1cc(-c2ccccc2)oc2cc(O)c(O)c(O)c12] | Baicalein | | |
| 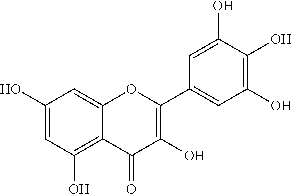 [SMILES: O=c1c(O)c(-c2cc(O)c(O)c(O)c2)oc2cc(O)cc(O)c12] | myricetin | | |
| 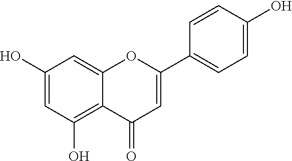 [SMILES: O=c1cc(-c2ccc(O)cc2)oc2cc(O)cc(O)c12] | apigenin | | |
| 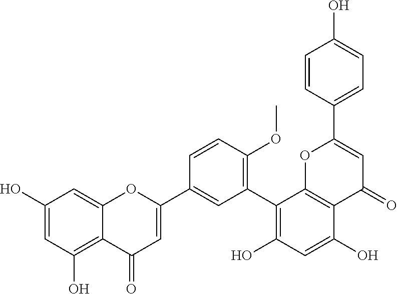 [SMILES: COc1ccc(-c2cc(=O)c3c(O)cc(O)cc3o2)cc1-c1c(O)cc(O)c2c(=O)cc(-c3ccc(O)cc3)oc12] | bilobetin | | |
| 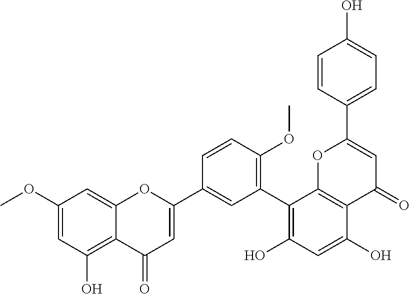 [SMILES: COc1cc(O)c2c(=O)cc(-c3ccc(OC)c(-c4c(O)cc(O)c5c(=O)cc(-c6ccc(O)cc6)oc45)c3)oc2c1] | Ginkgetin | | |
| 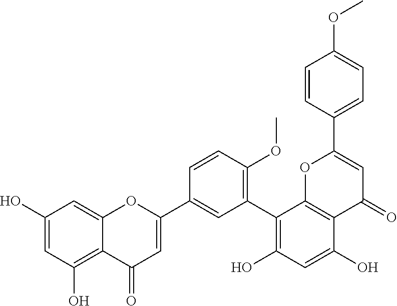 [SMILES: COc1ccc(-c2cc(=O)c3c(O)cc(O)c(-c4cc(-c5cc(=O)c6c(O)cc(O)cc6o5)ccc4OC)c3o2)cc1] | Isoginkgetin | | |
| 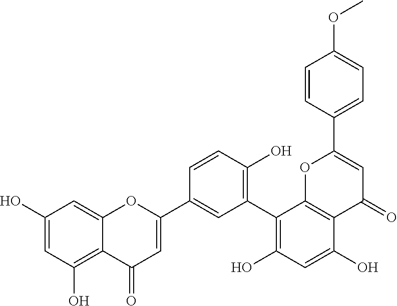 [SMILES: COc1ccc(-c2cc(=O)c3c(O)cc(O)c(-c4cc(-c5cc(=O)c6c(O)cc(O)cc6o5)ccc4O)c3o2)cc1] | Podocarpus flavone A | | |
| 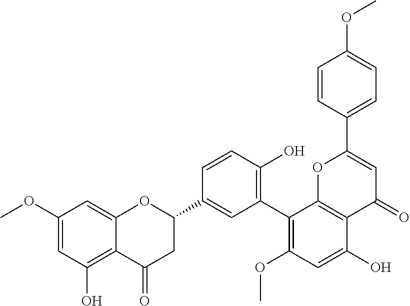 [SMILES: COc1ccc(-c2cc(=O)c3c(O)cc(OC)c(-c4cc([C@@H]5CC(=O)c6c(O)cc(OC)cc6O5)ccc4O)c3o2)cc1] | 2,3-dihydro- heveaflavone | | |
| 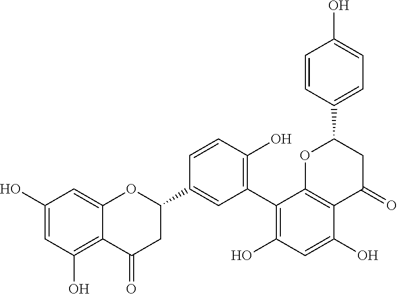 [SMILES: O=C1C[C@@H](c2ccc(O)c(-c3c(O)cc(O)c4c3O[C@H](c3ccc(O)cc3)CC4=O)c2)Oc2cc(O)cc(O)c21] | Tetrahydro- amentoflavone | | |
| 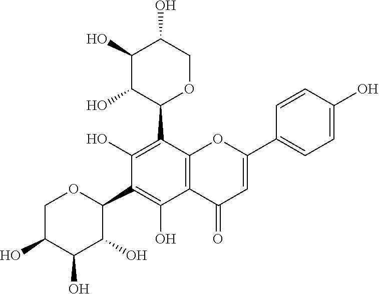 [SMILES: O=c1cc(-c2ccc(O)cc2)oc2c([C@@H]3OC[C@@H](O)[C@H](O)[C@H]3O)c(O)c([C@@H]3OC[C@H](O)[C@H](O)[C@H]3O)c(O)c12] | Apigenin 6-C-alpha-L- arabinopyranosyl-8-C- beta-D-xylopyranoside | | |
| 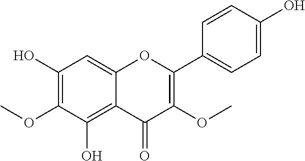 [SMILES: COc1c(O)cc2oc(-c3ccc(O)cc3)c(OC)c(=O)c2c1O] | 3,6-Dimethoxyapigenin | | |
| 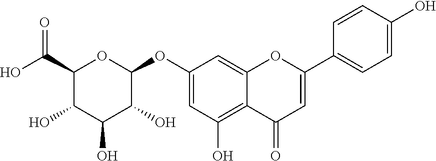 [SMILES: O=C(O)[C@H]1O[C@@H](Oc2cc(O)c3c(=O)cc(-c4ccc(O)cc4)oc3c2)[C@H](O)[C@@H](O)[C@@H]1O] | Apigenin 7-glucuronide | | |
| 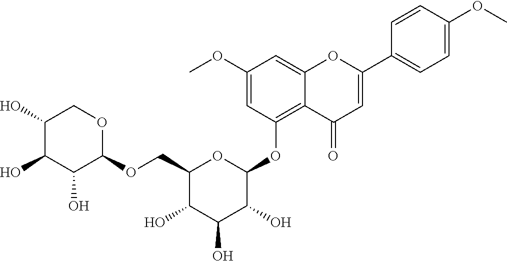 [SMILES: COc1ccc(-c2cc(=O)c3c(O[C@@H]4O[C@H](CO[C@@H]5OC[C@@H](O)[C@H](O)[C@H]5O)[C@@H](O)[C@H](O)[C@H]4O)cc(OC)cc3o2)cc1] | 7,4′-Di-O- methylapigenin 5-O- xylosylglucoside | | |
| 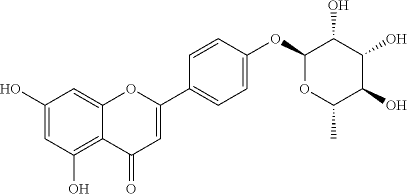 [SMILES: C[C@@H]1O[C@@H](Oc2ccc(-c3cc(=O)c4c(O)cc(O)cc4o3)cc2)[C@H](O)[C@H](O)[C@H]1O] | Apigenin 4′-O- rhamnoside | | |
| 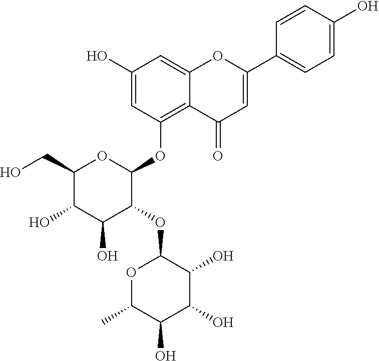 [SMILES: C[C@@H]1O[C@@H](O[C@H]2[C@H](Oc3cc(O)cc4oc(-c5ccc(O)cc5)cc(=O)c34)O[C@H](CO)[C@@H](O)[C@@H]2O)[C@H](O)[C@H](O)[C@H]1O] | Apigenin 5-O- neohesperidoside | | |
| 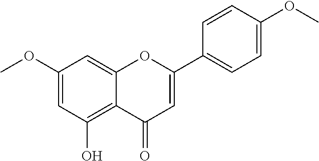 | 7,4′-Di-O- methylapigenin | | |
| 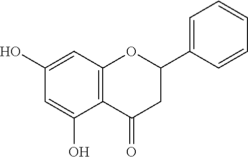 [SMILES: O=C1CC(c2ccccc2)Oc2cc(O)cc(O)c21] | Pinocembrin | | |

### Example 2—Flavonoid Mpro inhibitors

4 compounds with IC₅₀ values of 5-15 μM were identified, including amentoflavone, 3,8′-biapigenin, pinocembrin 7-O-(3″-galloyl-4″,6″-(S)-hexahydroxydiphenoyl)-beta-D-glucose (PGHG), and jaceidin triacetate (see structures in the table above).

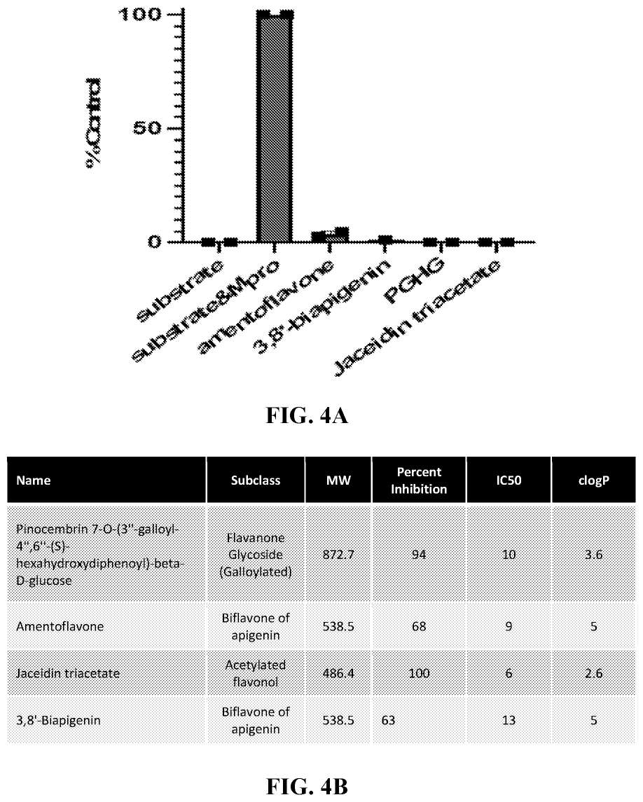

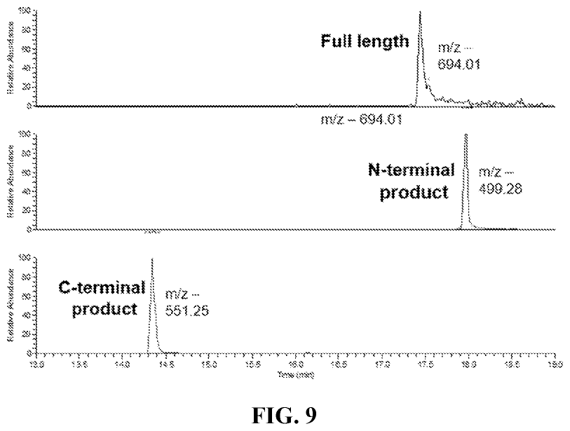

To confirm the Mpro-inhibiting potential of these compounds, they were further evaluated in a label free assay (FIG. 9B). Specifically, the cleavage of the substrate by mass spectrometry was quantified, monitoring the loss of full-length substrate and production of cleavage products. This assay demonstrated that at 50 μM concentration, amentoflavone, 3,8′-biapigenin, pinocembrin 7-O-(3″-galloyl-4″,6″-(S)-hexahydroxydiphenoyl)-beta-D-glucose, and jaceidin triacetate completely inhibited Mpro activity (FIG. 4A). Characteristics of these compounds is provided in FIG. 4B.

### Example 3—Biflavones of Apigenin inhibit Mpro

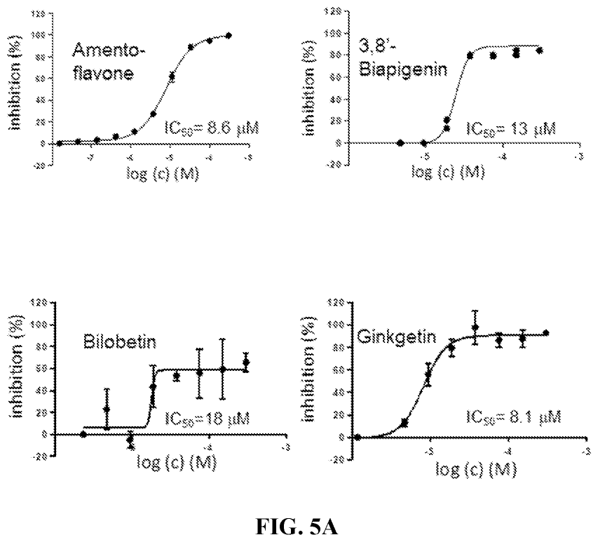

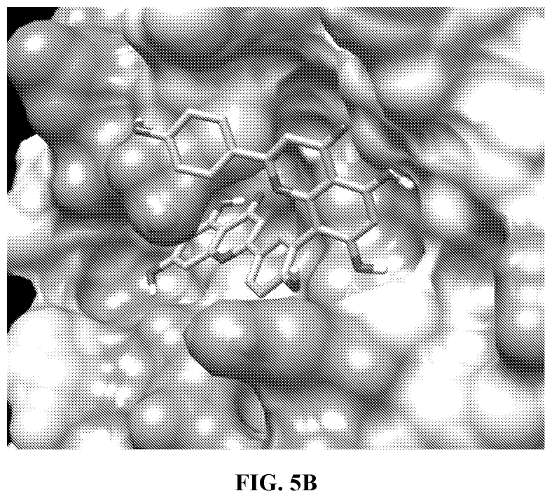

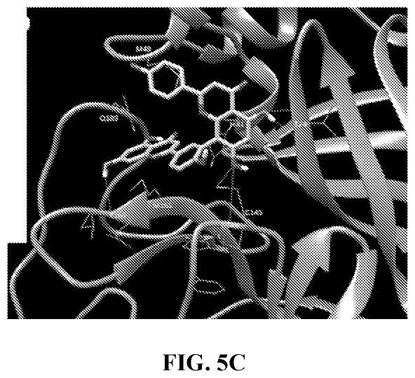

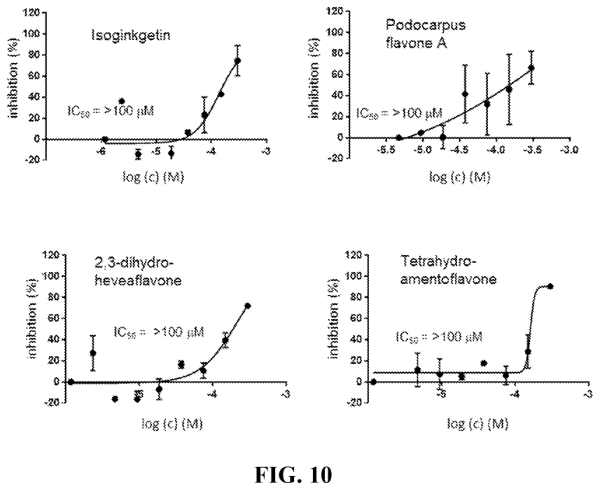

Bilobetin and ginkgetin (chemical structures shown in Table above) showed Mpro inhibitory activity comparable to amentoflavone (FIG. 5A), showing that biflavones capable of inhibiting Mpro. IC_{50S} of isoginkgetin, podocarpus flavone A, 2,3-dihydro-heveaflavone, and tetrahydro-amentoflavone are shown in FIG. 10.

Docking study (Autodock Vina) was conducted to model the interactions between amentoflavone and the catalytic domain of Mpro utilizing the reported high resolution crystal structure of Mpro (PDB Code: 7B3E). The top binding pose (delta E: 8.5 kcal/mol) from the simulation indicated that amentoflavone was able to occupy the substrate binding site of Mpro to inhibit its enzymatic activity (FIG. 5B). The formation of multiple hydrogen bonds (H-bond) between the inhibitor and protein contributed to the stabilization of the structural complex (FIG. 5C). Notably, the OH group at the 7″ position formed a H-bond with the mainchain NH of Gly143 (2,39 Å) and a sidechain SH (3.58 Å) of Cys145, respectively. Gly143 and Cys145, together with Ser144, constitute the ‘oxyanion hole’ of this cysteine protease and perform a pivotal role in catalyzing the hydrolysis of protein substrate. Moreover, H-bond interactions could be observed between the 7-OH and mainchain carbonyl (C═O) (2.30 Å) of Glu166, as well as between 4″-OH and mainchain NH (2.04 Å) or C═O (2.57 Å) of Thr26. Additional inhibitor-protein interactions that also contributed to the stabilization of the structural complex include NH₂-pi-interaction with Gln189, and hydrophobic interactions with Met49 and Met165.

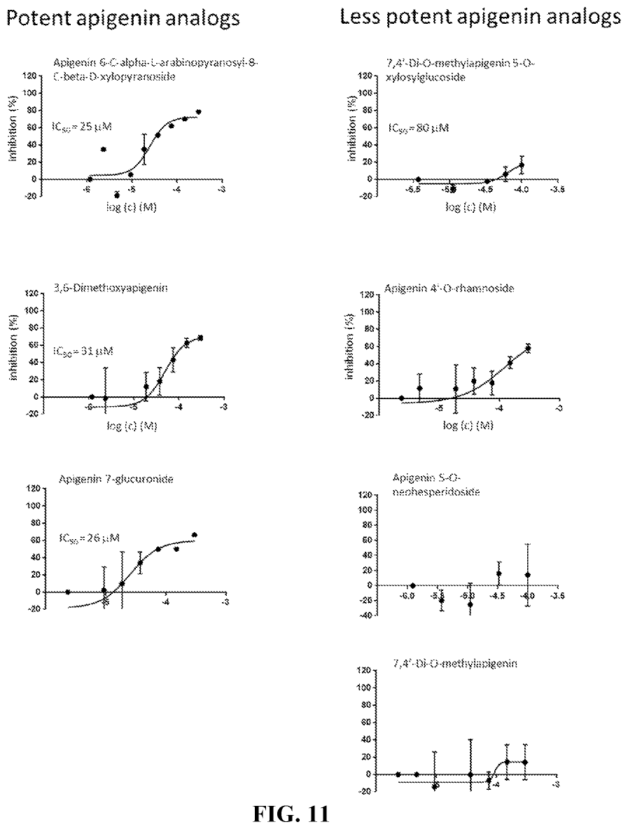

Amentoflavone (3′,8″-biapigenin) and 3,8′-biapigenin are formed from conjugation of two apigenin molecules. Follow-up assays confirmed the ability of apigenin to inhibit Mpro (IC₅₀=17.4+12.7 μM). Apigenin is among the most abundant flavones in the plant kingdom and is readily available in a purified form as an OTC nutritional supplement. Evaluation of apigenin analogs revealed a few compounds with inhibition against Mpro (FIG. 11). Several other apigenin analogs showed lesser activity against Mpro (FIG. 11).

### Example 4—Mode of Mpro inhibition differs among flavonoids

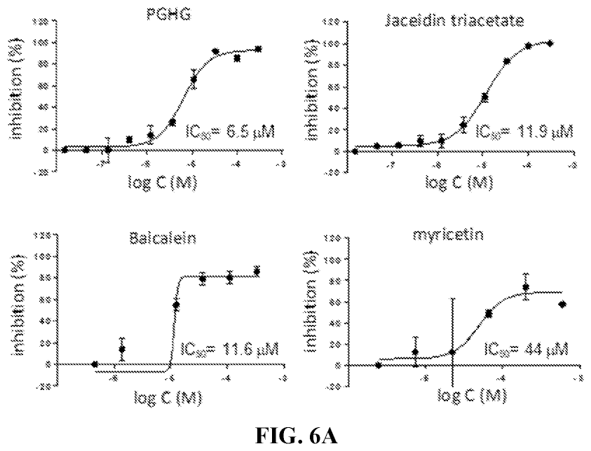

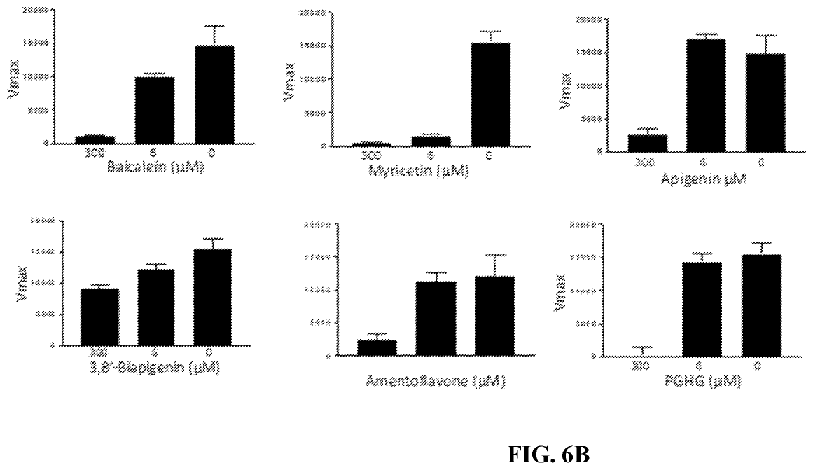

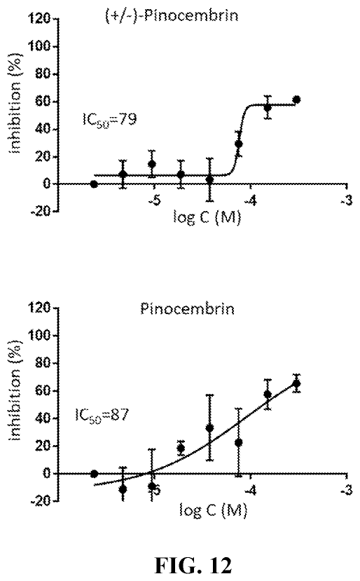

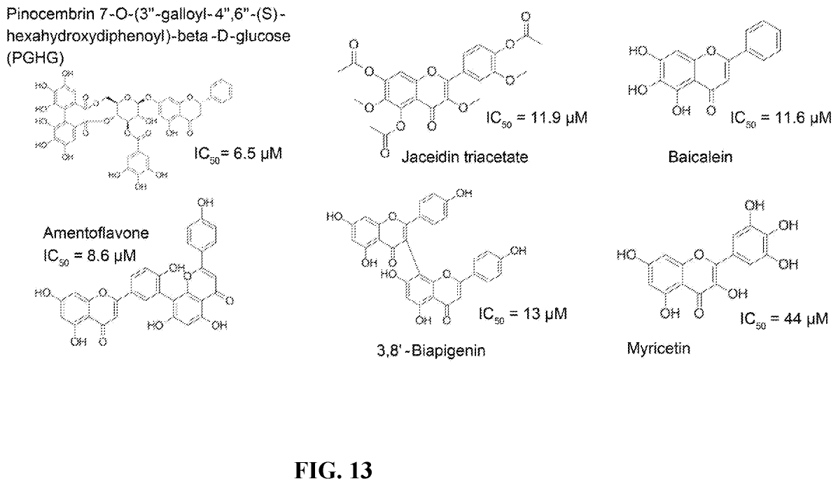

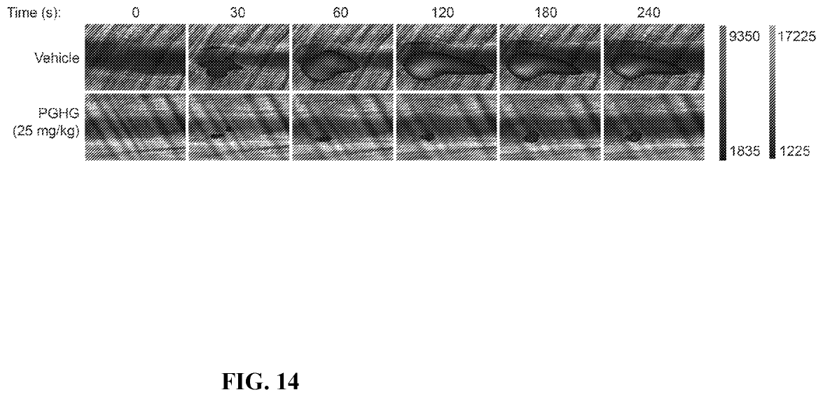

PGHG and jaceidin acetate were identified as inhibitors of SARS-CoV-2 Mpro. Both compounds showed inhibitory activity that was comparable to or better than the previously described Mpro inhibitors baicalein and myricetin (FIG. 6A). In contrast to PGHG, pinocembrin itself showed relatively poor inhibition of Mpro (FIG. 12). Jaceidin acetate was found to be toxic in Vero E6 cells at concentrations that inhibited Mpro and was not pursued further. Baicalein and myricetin showed inhibition in the initial screen at 17 μM, but did not make the original cut-off for further analysis.

Mpro can be blocked either by irreversible inhibitors that covalently bind the active site Cys145 or by reversible inhibitors that occlude the S1 binding pocket. High resolution structures from crystals of baicalein (PDB: 6M2N) and myricetin (PDB: 7B3E) in complex with SARS-CoV-2 Mpro demonstrate very different orientations of these flavonoids in the S1 binding pocket of Mpro despite their substantial structural similarity. Myricetin in complex with Mpro is predicted to become covalently bound to the active cysteine Cys145 at the 5′ position. In contrast, baicalein associates with Mpro non-covalently. To assess whether PGHG, apigenin and its analogs bind reversibly or irreversibly, jump dilution reversibility assays were performed. These studies showed that inhibition of myricetin was resistant to dilution, indicating an irreversible mechanism of binding, consistent with the crystal structure indicating a covalent interaction with Cys145 (FIG. 6B). In contrast, inhibition of Mpro by baicalein, apigenin, amentoflavone, and PGHG was completely reversible by dilution, indicating a reversible mechanism of inhibition (FIG. 6B).

### Example 5—Evaluation of Mpro inhibitors in a SARS-CoV-2 replication assay

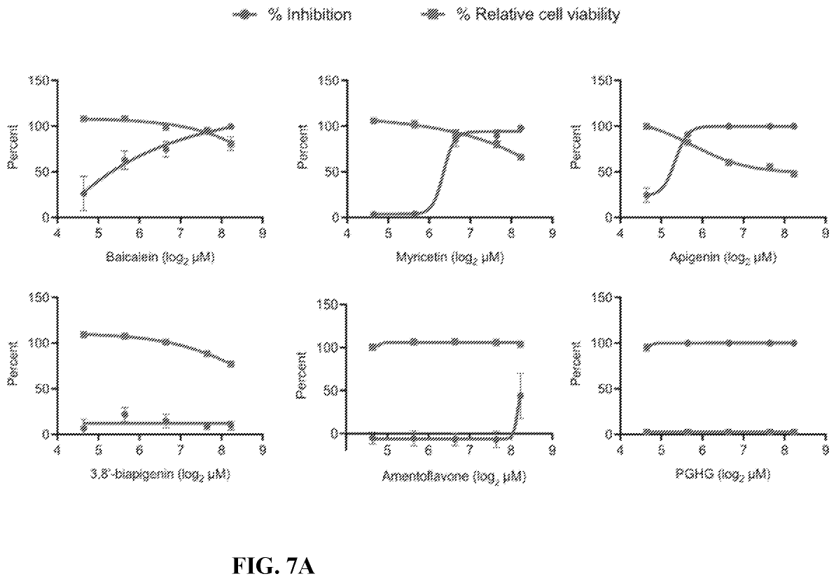

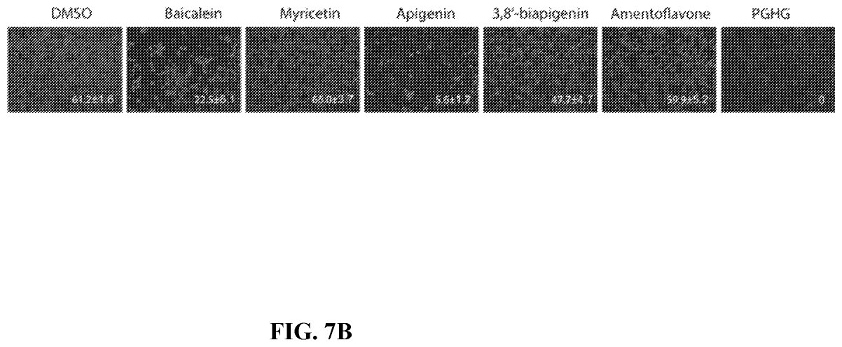

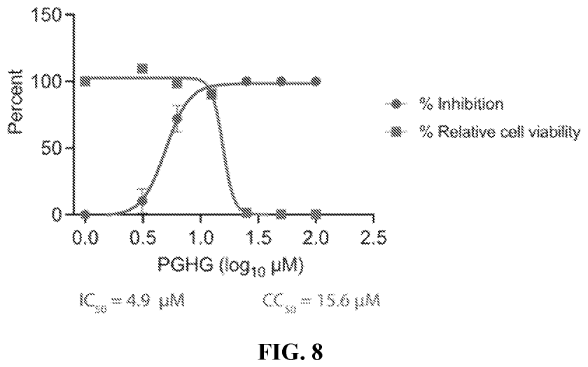

Exemplified flavonoids were tested in a Vero E6-based SARS-CoV-2 replication assay that relies on immunofluorescence microscopy to detect infected cells by staining for the viral nucleocapsid (N) protein. Evaluation of baicalein, myricetin, apigenin, amentoflavone, 3,8″-biapigenin, and PGHG demonstrated different activities of each of the compounds (FIG. 7A). These studies confirmed the antiviral activity of baicalein. Myricetin, which was previously reported not to have antiviral activity, showed inhibitory activity only at 100 μM. Apigenin showed activity that was somewhat better than baicalein at 50 μM (FIG. 7A,B). However, the biflavones showed little activity. The most potent flavonoid inhibitor of SARS-CoV-2 was PGHG (FIG. 7C), which showed almost complete inhibitory activity at 25 μM. Further evaluation of PGHG potency showed that it blocks SARS-CoV-2 viral replication with an IC₅₀ of ˜5 μM.

### Discussion of Examples 2-5

PGHG is a uniquely potent inhibitor of SARS-CoV-2 replication among flavonoids. PGHG is an ellagitannin composed of pinocembrin, hexahydroxydiphenic acid, and gallic acid linked to glucose (doi: 10.1186/s12906-019-2632-3; doi: 10.3390/molecules190811045). It is found in the aerial part of Penthorum chinense Pursh, which is widely distributed in eastern Asia and is used in China as dietotherapy for liver disease and for other indications (doi: 10.3390/molecules190811045; doi: 10.1039/c3fo60245a). In the examples of this disclosure, PGHG had relatively low autofluorescence, enabling its identification in a fluorescence-based enzymatic assay in which it demonstrated an IC₅₀ in the low micromolar range (FIG. 6). It showed significant inhibition of SARS-CoV-2 replication in the Vero E6-based assay (FIG. 7), with an IC₅₀ in the viral replication assay of 5 μM, very close to its IC₅₀ for inhibition of Mpro. Some toxicity was observed in Vero 6E cells at higher concentrations; however, doses that were not associated with toxicity were able to inhibit SARS-CoV-2 replication. PGHG is tolerated in mice at 25 mg/kg. Clinically, there is substantial experience with human ingestion of chinense pursh extract, but little is known about the clinical effects of purified PGHG consumption in humans.

In contrast to PGHG, apigenin is an extremely common flavonoid, considered among the five most ubiquitous flavonoids in the plant kingdom (doi: 10.1155/2019/7010467; doi: 10.1080/17425255.2017.1251903). It is particularly abundant in commonly consumed foods such as celery, parsley, and chamomile (doi: 10.1080/17425255.2017.1251903). Purified preparations of apigenin (98% pure) are readily available and we found that such a preparation extracted from chamomile inhibited Mpro with the same potency as preparations from scientific supply companies. Apigenin was somewhat less potent at inhibiting Mpro than its biflavones, amentoflavone and 3,8′-biapigenin. Docking studies of amentoflavone suggested that it bound to Mpro by forming some of the key binding interactions as observed in the cocrystal structures of Mpro in complex with baicalein or myricetin, including H-bonds with Gly143 and Cys145 in the ‘oxyanion hole’ of the catalytic reaction site in the cysteine protease. However, the biflavones had less activity in the SARS-CoV-2 viral replication assay. While apigenin shows some toxicity in cell culture, it inhibits viral replication at concentrations at doses that are not toxic. Oral dosing of apigenin may achieve tissue levels that inhibit SARS-CoV-2 replication in animals.

Baicalein and myricetin have previously been identified as inhibitors of Mpro. Baicalein was identified in an evaluation of a Shuanghuanglian preparation for the ability to inhibit Mpro (doi.org/10.1038/s41401-020-0483-6). It was also found to inhibit SARS-CoV-2 replication. Evaluation of baicalein mechanism of action in SARS-CoV-2 replication indicated that, in addition to inhibiting Mpro, it interferes with oxidative phosphorylation in a manner dependent on the mitochondrial permeability pore and this activity could interfere with SARS-CoV-2 replication (doi.org/10.1038/s41392-020-00353-x). Although instant experiments show less potent IC_{50S} in both the Mpro and viral replication assays, perhaps owing to differences in techniques, the results are largely consistent. Baicalein is currently the flavonoid Mpro inhibitor to be tested in vivo for anti-SARS-CoV-2 effect. Using a human ACE2 transgenic mouse model, Song et al. showed that 50-200 mg/kg of the crystal form p of baicalein prevented reduction in body weight and decreased lung injury resulting from SARS-CoV-2 infection (doi.org/10.1016/j.bcp.2020.114302). Myricetin was initially identified in a virtual screen of 8,700 compounds for inhibitors of Mpro. It was subsequently shown to inhibit Mpro in an enzymatic assay and X-ray crystal structure in complex with Mpro indicated covalent binding to Cys145 (doi.org/10.1101/2020.12.16.422677;). Although this study did not find inhibition of SARS-CoV-2 replication by myricetin, it was find that myricetin inhibits SARS-CoV-2 replication under the conditions of the current assay (FIG. 7), albeit somewhat less potently than PGHG, baicalein, or apigenin.

While the combination of enzymatic and molecular modeling studies provide strong evidence that these compounds interfere with Mpro activity. However, flavonoids are promiscuous compounds and it is possible that they interfere with SARS-CoV-2 replication by additional or alternative activities. With regard to their clinical development, a limitation of flavonoid-based therapies is their bioavailability. Although inexpensive and widely available, it is not easy to predict the oral dosing that would be required to reach adequate tissue levels for antiviral activity.

### Example 6—antithrombotic activity

PGHG was found to be a potent PDI inhibitor, which blocked PDI reductase activity in an insulin turbidimetric assay with an IC50=3.99+/−1.14 μM and in a di-eosin-GSSG assay with an IC50=1.50+/−0.60 μM. When tested against isolated fragments of PDI, PGHG inhibited isolated a and a′ fragments as well as ab, b‘xa’ and abb′x fragments, indicating that it acts on the a and a′ domains of PDI. Since PDI is essential for thrombosis, PGHG blocks platelet accumulation and fibrin formation following vascular injury. Mice was infused with 25 mg/kg PGHG or vehicle and subsequently induced thrombus formation via laser-induced injury of an arteriole within the cremaster circulation. Infusion of PGHG resulted in a 82+/−6.2% inhibition of platelet accumulation and a 79+/−3.7% inhibition of fibrin formation. In contrast 25 mg/kg had no significant effect on tail bleeding in mice compared to vehicle control. Targeted therapies remain an important component of the armamentarium against COVID19. The present results show that a naturally occurring flavonoid, PGHG, found in Penthorum chinense Pursh, inhibits both SARS-CoV-2 replication and thrombosis without enhancing bleeding. This observation provides proof-of-principle for the development of plant-based flavonoid therapies for inhibition of β-coronaviruses and supports the further evaluation of PGHG for therapeutic use in COVID19.

### OTHER EMBODIMENTS

It is to be understood that while the present application has been described in conjunction with the detailed description thereof, the foregoing description is intended to illustrate and not limit the scope of the present application, which is defined by the scope of the appended claims. Other aspects, advantages, and modifications are within the scope of the following claims.

1. A method of modulating activity of a main protease of a virus selected from SARS-CoV, SAR-CoV-2, and MERS-CoV in a cell, the method comprising contacting the cell with an effective amount of a compound of Formula (I): 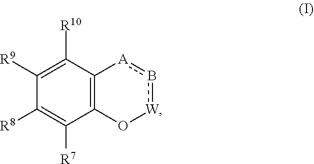
or a pharmaceutically acceptable salt thereof, wherein:
A is CR¹R²;
B is CR³R⁴;
W is CR⁵R⁶;
R¹, R², R³, R⁴, R⁵, R⁶, R⁷, R⁸, R⁹, and R¹⁰ are each independently selected from H, Cy^{A}, halo, CN, NO₂, C₁₋₆ alkyl, C₁₋₆ haloalkyl, C₄₋₁₅ alkenyl, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)Rb, NR^{c1}C(O)OR^{a1} NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl and C₄₋₁₅ alkenyl are each optionally substituted with 1, 2 or 3 substituents independently selected from Cy^{A}, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1} NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1} and S(O)₂NR^{c1}R^{d1};
each [symbol] bond is either a single bond or a double bond, provided that:
(i) when the bond between A and B is a double bond, then R² and R⁴ are absent and the bond between B and W is a single bond; and
(ii) when the bond between B and W is a double bond, then R⁴ and R⁶ are absent and the bond between A and B is a single bond;
or R¹ and R² together form an oxo group;
or R³ and R⁵, together with the carbon atoms to which they are attached, form 4-10 membered heterocycloalkyl, which is optionally substituted with 1, 2, 3, 4, or 5 substituents independently selected from R^{Cy};
or R¹ and R³, together with the carbon atoms to which they are attached, form 4-10 membered heterocycloalkyl, which is optionally substituted with 1, 2, 3, 4, or 5 substituents independently selected from R^{Cy};
R⁷ and R⁸, together with the carbon atoms to which they are attached, form a 4-10 membered heterocycloalkyl, which is optionally substituted with 1, 2, 3, or 4 substituents independently selected from R^{Cy};
R⁸ and R⁹, together with the carbon atoms to which they are attached, form a 4-10 membered heterocycloalkyl, which is optionally substituted with 1, 2, 3, or 4 substituents independently selected from R^{Cy};
R⁹ and R¹⁰, together with the carbon atoms to which they are attached, form a 4-10 membered heterocycloalkyl, which is optionally substituted with 1, 2, 3, or 4 substituents independently selected from R^{Cy};
each R^{a1}, R^{b1}, R^{c1}, and R^{d1} is independently selected from H, Cy^{A}, C₁₋₆ alkyl, C₄₋₁₅ alkenyl, C₁₋₆ haloalkyl, wherein said C₁₋₆ alkyl and C₄₋₁₅ alkenyl are each optionally substituted with 1, 2, or 3 substituents independently selected from R^{g};
each Cy^{A} is independently selected from C₆₋₁₀ aryl, C₃₋₈ cycloalkyl, and 4-10 membered heterocycloalkyl, each of which is optionally substituted with 1, 2, 3, 4, or 5 substituents independently selected from R^{Cy};
each R^{Cy} is independently selected from halo, C₁₋₆ alkyl, C₁₋₆ haloalkyl, C₆₋₁₀ aryl, C₄₋₁₅ alkenyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2} NR^{c2}C(O)R^{b2}, NR^{c2}C(O)OR^{a2} NR^{c2}C(O)NR^{c2}R^{d2}, NR^{c2}S(O)R^{b2}, NR^{c2}S(O)₂R^{b2} NR^{c2}S(O)₂NR^{c2}R^{d2}, S(O)R^{b2}, S(O)NR^{c2}R^{d2}, S(O)₂R^{b2}, and S(O)₂NR^{c2}R^{d2}, wherein said C₁₋₆ alkyl, C₄₋₁₅ alkenyl, and C₆₋₁₀ aryl are each optionally substituted by 1, 2, or 3 substituents independently selected from C₁₋₆ alkyl, C₁₋₆ haloalkyl, halo, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2} NR^{c2}C(O)R^{b2}, NR^{c2}C(O)OR^{a2}, NR^{c2}C(O)NR^{c2}R^{d2} NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2} S(O)₂R^{b2}, and S(O)₂NR^{c2}R^{d2};
each R^{a2}, R^{b2}, R^{c2}, and R^{d2} is independently selected from H, C₁₋₆ alkyl, C₄₋₁₅ alkenyl, C₆₋₁₀ aryl, and 4-10 membered heterocycloalkyl, each of which is optionally substituted with 1, 2, 3, 4, or 5 substituents independently selected from R^{A};
each R^{A} is independently selected from C₁₋₆ alkyl, C₁₋₆ haloalkyl, halo, CN, NO₂, OR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}, wherein said C₁₋₆ alkyl is optionally substituted with 1, 2, or 3 substituents selected from CN, NO₂, OR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3};
each R^{a3}, R^{b3}, R^{c3}, and R^{d3} is independently selected from H, C₁₋₆ alkyl, C₄₋₁₅ alkenyl, C₁₋₆ haloalkyl, wherein said C₁₋₆ alkyl and C₄₋₁₅ alkenyl are each optionally substituted with 1, 2, or 3 substituents independently selected from R^{g};
or any R^{c1} and R^{d1} together with the N atom to which they are attached form a 4-, 5-, 6-, or 7-membered heterocycloalkyl group optionally substituted with 1, 2, or 3 substituents independently selected from R^{g};
or any R^{c2} and R^{d2} together with the N atom to which they are attached form a 4-, 5-, 6-, or 7-membered heterocycloalkyl group optionally substituted with 1, 2, or 3 substituents independently selected from R^{g};
or any R^{c3} and R^{d3} together with the N atom to which they are attached form a 4-, 5-, 6-, or 7-membered heterocycloalkyl group optionally substituted with 1, 2, or 3 substituents independently selected from R^{g}; and
each R^{g} is independently selected from OH, NO₂, CN, halo, C₁₋₆ alkyl, C₁₋₄ haloalkyl, C₁₋₆ alkoxy, C₁₋₆ haloalkoxy, cyano-C₁₋₃ alkylene, HO—C₁₋₃ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino;
provided that the compound of Formula (I) comprises at least one C₄₋₁₅ alkenyl.

2-3. (canceled)

4. The method of claim 3, wherein R¹, R², R³, R⁴, R⁵, R⁶, R⁷, R⁸, R⁹, and R¹⁰ are each independently selected from H, Cy^{A}, C₁₋₆ alkyl, C₄₋₁₅ alkenyl, and OR^{a1}, wherein said C₁₋₆ alkyl and C₄₋₁₅ alkenyl are each optionally substituted with OR^{a1}.

5. The method of claim 1, wherein each R^{a1}, R^{b1}, R^{c1}, and R^{d1} is independently selected from H, Cy^{A}, C₁₋₆ alkyl, and C₄₋₁₅ alkenyl.

6. The method of claim 1, wherein each Cy^{A} is independently selected from C₆₋₁₀ aryl, C₃₋₈ cycloalkyl, and 4-10 membered heterocycloalkyl, each of which is optionally substituted with 1, 2, or 3 substituents independently selected from R^{Cy}.

7. (canceled)

8. The method of claim 1, wherein each R^{Cy} is independently selected from C₁₋₆ alkyl, C₆₋₁₀ aryl, C₄₋₁₅ alkenyl, OR^{a2}, C(O)R^{b2}, and OC(O)R^{b2}, wherein said C₁₋₆ alkyl, C₄₋₁₅ alkenyl, and C₆₋₁₀ aryl are each optionally substituted by 1, 2, or 3 substituents independently selected from OR^{a2} and OC(O)R^{b2}.

9. The method of claim 1, wherein each R^{a2}, R^{b2}R^{c2}, and R^{d2} is independently selected from H, C₁₋₆ alkyl, C₄₋₁₅ alkenyl, C₆₋₁₀ aryl, and 4-10 membered heterocycloalkyl, each of which is optionally substituted with 1, 2, or 3 substituents independently selected from R^{A}.

10. (canceled)

11. The method of claim 1, wherein each R^{A} is independently selected from C₁₋₆ alkyl, OR^{a3}, and OC(O)R^{b3}, wherein said C₁₋₆ alkyl is optionally substituted with 1, 2, or 3 substituents selected from OR^{a3} and OC(O)R^{b3}.

12. The method of claim 1, wherein each R^{a3}, R^{b3}, R^{c3}, and R^{d3} is independently selected from H, C₁₋₆ alkyl, and C₄₋₁₅ alkenyl, wherein said C₁₋₆ alkyl and C₄₋₁₅ alkenyl are each optionally substituted with 1, 2, or 3 substituents independently selected from R^{g}.

13. The method of claim 1, wherein each R^{g} is independently selected from OH, C₁₋₆ alkyl, C₁₋₆ alkoxy, HO—C₁₋₃ alkylene, amino, C₁₋₆ alkylamino, and di(C₁₋₆ alkyl)amino.

14-15. (canceled)

16. The method of claim 1, wherein the compound of Formula (I) is has formula: 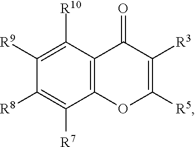
or a pharmaceutically acceptable salt thereof.

17. The method of claim 16, wherein the compound has formula: 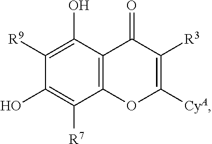
or a pharmaceutically acceptable salt thereof.

18. The method of claim 16, wherein the compound has formula: 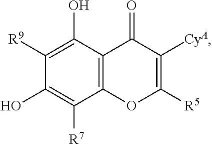
or a pharmaceutically acceptable salt thereof.

19-24. (canceled)

25. The method of claim 1, wherein the compound of Formula (I) is selected from any one of the compounds listed in any one of Tables 1a-2e or Table 3, or a pharmaceutically acceptable salt thereof.

26. (canceled)

27. A method of treating or preventing a viral infection caused by a coronavirus selected from SARS-CoV, SAR-CoV-2, and MERS-CoV in a subject, the method comprising administering to the subject a therapeutically effective amount of a compound of Formula (I), as recited in claim 1, or a pharmaceutically acceptable salt thereof.

28. The method of claim 27, wherein the viral infection is coronavirus disease 2019 (COVID-19).

29-31. (canceled)

32. A method of treating or preventing a viral infection caused by a coronavirus selected from SARS-CoV, SAR-CoV-2, and MERS-CoV in a subject, the method comprising intranasally administering to the subject in need thereof a therapeutically effective amount of a compound of Formula (I): 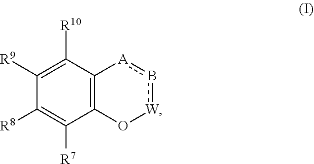
or a pharmaceutically acceptable salt thereof, wherein:
A is CR¹R²;
B is CR³R⁴;
W is CR⁵R⁶;
R¹, R², R³, R⁴, R⁵, R⁶, R⁷, R⁸, R⁹, and R¹⁰ are each independently selected from H, Cy^{A}, halo, CN, NO₂, C₁₋₆ alkyl, C₁₋₆ haloalkyl, C₄₋₁₅ alkenyl, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1} NR^{c1}C(O)OR^{a1} NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl and C₄₋₁₅ alkenyl are each optionally substituted with 1, 2 or 3 substituents independently selected from Cy^{A}, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{a1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1} and S(O)₂NR^{c1}R^{d1};
each [symbol] bond is either a single bond or a double bond, provided that:
(iii) when the bond between A and B is a double bond, then R² and R⁴ are absent and the bond between B and W is a single bond; and
(iv) when the bond between B and W is a double bond, then R⁴ and R⁶ are absent and the bond between A and B is a single bond;
or R¹ and R² together form an oxo group;
or R³ and R⁵, together with the carbon atoms to which they are attached, form 4-10 membered heterocycloalkyl, which is optionally substituted with 1, 2, 3, 4, or 5 substituents independently selected from R^{Cy};
or R¹ and R³, together with the carbon atoms to which they are attached, form 4-10 membered heterocycloalkyl, which is optionally substituted with 1, 2, 3, 4, or 5 substituents independently selected from RC;
R⁷ and R⁸, together with the carbon atoms to which they are attached, form a 4-10 membered heterocycloalkyl, which is optionally substituted with 1, 2, 3, or 4 substituents independently selected from R^{Cy};
R⁸ and R⁹, together with the carbon atoms to which they are attached, form a 4-10 membered heterocycloalkyl, which is optionally substituted with 1, 2, 3, or 4 substituents independently selected from R^{Cy};
R⁹ and R¹⁰, together with the carbon atoms to which they are attached, form a 4-10 membered heterocycloalkyl, which is optionally substituted with 1, 2, 3, or 4 substituents independently selected from RC;
each R^{a1}, R^{b1}, R^{c1}, and R^{d1} is independently selected from H, Cy^{A}, C₁₋₆ alkyl, C₄₋₁₅ alkenyl, C₁₋₆ haloalkyl, wherein said C₁₋₆ alkyl and C₄₋₁₅ alkenyl are each optionally substituted with 1, 2, or 3 substituents independently selected from R^{g};
each Cy^{A} is independently selected from C₆₋₁₀ aryl, C₃₋₈ cycloalkyl, and 4-10 membered heterocycloalkyl, each of which is optionally substituted with 1, 2, 3, 4, or 5 substituents independently selected from R^{Cy};
each R^{Cy} is independently selected from halo, C₁₋₆ alkyl, C₁₋₆ haloalkyl, C₆₋₁₀ aryl, C₄₋₁₅ alkenyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NRe^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}, wherein said C₁₋₆ alkyl, C₄₋₁₅ alkenyl, and C₆₋₁₀ aryl are each optionally substituted by 1, 2, or 3 substituents independently selected from C₁₋₆ alkyl, C₁₋₆ haloalkyl, halo, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2} NR^{c2}C(O)R^{b2}, NR²C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
each R², R^{b2}, R^{c2}, and R^{d2} is independently selected from H, C₁₋₆ alkyl, C₄₋₁₅ alkenyl, C₆₋₁₀ aryl, and 4-10 membered heterocycloalkyl, each of which is optionally substituted with 1, 2, 3, 4, or 5 substituents independently selected from R^{A};
each R^{A} is independently selected from C₁₋₆ alkyl, C₁₋₆ haloalkyl, halo, CN, NO₂, OR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3} NR^{c3}C(O)R^{b3} NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}, wherein said C₁₋₆ alkyl is optionally substituted with 1, 2, or 3 substituents selected from CN, NO₂, OR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3} NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3};
each R^{a3}, R^{b3}, R^{c3}, and R^{d3} is independently selected from H, C₁₋₆ alkyl, C₄₋₁₅ alkenyl, C₁₋₆ haloalkyl, wherein said C₁₋₆ alkyl and C₄₋₁₅ alkenyl are each optionally substituted with 1, 2, or 3 substituents independently selected from R^{g};
or any R^{c1} and R^{d1} together with the N atom to which they are attached form a 4-, 5-, 6-, or 7-membered heterocycloalkyl group optionally substituted with 1, 2, or 3 substituents independently selected from R^{g};
or any R^{c2} and R^{d2} together with the N atom to which they are attached form a 4-, 5-, 6-, or 7-membered heterocycloalkyl group optionally substituted with 1, 2, or 3 substituents independently selected from R^{g};
or any R^{c3} and R^{d3} together with the N atom to which they are attached form a 4-, 5-, 6-, or 7-membered heterocycloalkyl group optionally substituted with 1, 2, or 3 substituents independently selected from R^{g}; and
each R^{g} is independently selected from OH, NO₂, CN, halo, C₁₋₆ alkyl, C₁₋₄ haloalkyl, C₁₋₆ alkoxy, C₁₋₆ haloalkoxy, cyano-C₁₋₃ alkylene, HO—C₁₋₃ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

33. The method of claim 32, wherein the compound of Formula (I) is selected from quercetin, luteolin, baicalin, amentoflavone, hesperetin, hesperidin, hervacetin, rhoifolin, pectolinarin, gallocatechin, gallocatechin gallate, diosmin, kaempferol, luteolin-7-glucoside, quercetin-3-β-galactoside, naringenin, apigenin-7-glucoside, catechin, pigallocatechin, isoquercetin, epigallocatechin, herbacetin, isotheaflavin, baicalein, theacitrin A, corilagin, theaflavin, (-)-taxifolin, Rhamnetin, quercetin 3-glucuronide-7-glucoside, quercetin 3-vicianoside, delphinidin 3-O-glucoside, petunidin 3-O-glucoside, chrysoeriol 8-C-glucoside, schaftoside, rutin, hypericin, cyanidin 3-glucoside, glabridin, orientin, astilbin, puerarin, astragalin, 2′-hydroxygenistein, genistein, morin, dihydromorin, steppogenin, 6-methoxyluteolin, pinocembrin 7-O-(3″-galloyl-4″,6″-(S)-hexahydroxydiphenoyl)-β-D-glucose, biorobin, jaceidin triacetate, kaempferol-4′-O-beta-D-glucopyranoside, dracoflavan B1, isoschaftoside, norartocarpetin, 3′,4′,5,7-tetra hydroxyisoflavanone, and artocarpanone, or a pharmaceutically acceptable salt thereof.

34. The method of claim 32, wherein the compound of Formula (I) is selected from pinocembrin 7-O-(3″-galloyl-4″,6″-(S)-hexahydroxydiphenoyl)-β-D-glucose (PGHG), Amentoflavone, Jaceidin triacetate, 3,8′-Biapigenin, Baicalein, myricetin, apigenin, bilobetin, Ginkgetin, Isoginkgetin, Podocarpus flavone A, 2,3-dihydro-heveaflavone, Tetrahydro-amentoflavone, Apigenin 6-C-alpha-L-arabinopyranosyl-8-C-beta-D-xylopyranoside, 3,6-Dimethoxyapigenin, Apigenin 7-glucuronide, 7,4′-Di-O-methylapigenin 5-O-xylosylglucoside, Apigenin 4′-O-rhamnoside, Apigenin 5-O-neohesperidoside, 7,4′-Di-O-methylapigenin, and Pinocembrin.

35. The method of claim 32, wherein the viral infection is coronavirus disease 2019 (COVID-19).

36-40. (canceled)

41. A method of inhibiting thrombosis in a subject, the method comprising administering to the subject a therapeutically effective amount of a compound of Formula (I) as recited in claim 1, or a pharmaceutically acceptable salt thereof.
